(19) United States
(12) Patent Application Publication
(10) Pub. No.: US 2009/0221567 A1
(43) Pub. Date: Sep. 3, 2009

(54) MUSCARINIC RECEPTOR AGONISTS, COMPOSITIONS, METHODS OF TREATMENT THEREOF, AND PROCESSES FOR PREPARATION THEREOF 177

(75) Inventors: Yun-Xing Cheng, Saint Laurent (CA); Xuehong Luo, Saint Laurent (CA); Mehrnaz Pourashraf, Saint Laurent (CA); Vijayaratnam Santhakumar, Saint Laurent (CA); Miroslaw Jerzy Tomaszewski, Saint Laurent (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/392,219

(22) Filed: Feb. 25, 2009

Related U.S. Application Data
(63) Continuation of application No. 61/032,138, filed on Feb. 28, 2008.

(51) Int. Cl. A61K 31/536; C07D 401/14; C07D 413/14; A61K 31/517; A61P 25/18; A61P 25/28; A61P 29/00; A61P 25/22; A61P 25/24

(52) U.S. Cl. 514/230.5; 544/286; 544/105; 544/92; 514/266.22

(57) ABSTRACT

Compounds of Formula I, or pharmaceutically acceptable salts thereof:

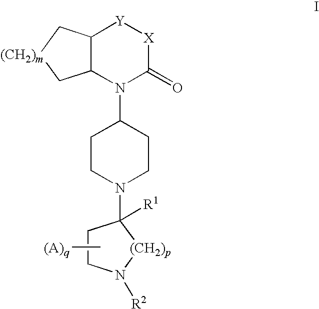

wherein Y, X, A, R¹, R², m, p, and q are as defined in the specification as well as salts and pharmaceutical compositions including the compounds are prepared. They are useful in therapy, in particular in the management of pain.

### BACKGROUND OF THE INVENTION

1. Field of the Invention

The present invention relates to agonists of muscarinic receptors. The present invention also provides compositions comprising such agonists, and methods therewith for treating muscarinic receptor mediated diseases. Particularly, the present invention is related to compounds that may be effective in treating pain, Alzheimer's disease, and/or schizophrenia.

2. Discussion of Technology

The neurotransmitter acetylcholine binds to two types of cholinergic receptors: the ionotropic family of nicotinic receptors and the metabotropic family of muscarinic receptors. Muscarinic receptors belong to the large superfamily of plasma membrane-bound G protein coupled receptors (GPCRs) and show a remarkably high degree of homology across species and receptor subtype. These M1-M5 muscarinic receptors are predominantly expressed within the parasympathetic nervous system which exerts excitatory and inhibitory control over the central and peripheral tissues and participate in a number of physiologic functions, including heart rate, arousal, cognition, sensory processing, and motor control.

Muscarinic agonists such as muscarine and pilocarpine, and antagonists, such as atropine have been known for over a century, but little progress has been made in the discovery of receptor subtype-selective compounds, thereby making it difficult to assign specific functions to the individual receptors. See, e.g., DeLapp, N. et al., “Therapeutic Opportunities for Muscarinic Receptors in the Central Nervous System,” J. Med. Chem., 43(23), pp. 4333-4353 (2000); Hulme, E. C. et al., “Muscarinic Receptor Subtypes,” Ann. Rev. Pharmacol. Toxicol., 30, pp. 633-673 (1990); Caulfield, M. P. et al., “Muscarinic Receptors-Characterization, Coupling, and Function,” Pharmacol. Ther., 58, pp. 319-379 (1993); Caulfield, M. P. et al., International Union of Pharmacology. XVII. Classification of Muscarinic Acetylcholine Receptors,” Pharmacol. Rev., 50, pp. 279-290 (1998).

The Muscarinic family of receptors is the target of a large number of pharmacological agents used for various diseases, including leading drugs for COPD, asthma, urinary incontinence, glaucoma, schizophrenia, Alzheimer's (AchE inhibitors), and Pain.

For example, direct acting muscarinic receptor agonists have been shown to be antinociceptive in a variety of animal models of acute pain (Bartolini A., Ghelardini C., Fantetti L., Malcangio M., Malmberg-Aiello P., Giotti A. Role of muscarinic receptor subtypes in central antinociception. Br. J. Pharmacol. 105:77-82, 1992.; Capone F., Aloisi A. M., Carli G., Sacerdote P., Pavone F. Oxotremorine-induced modifications of the behavioral and neuroendocrine responses to formalin pain in male rats. Brain Res. 830:292-300, 1999.).

A few studies have examined the role of muscarinic receptor activation in chronic or neuropathic pain states. In these studies, the direct and indirect elevation of cholinergic tone was shown to ameliorate tactile allodynia after intrathecal administration in a spinal ligation model of neuropathic pain in rats and these effects again were reversed by muscarinic antagonists (Hwang J.-H., Hwang K.-S., Leem J.-K., Park P.-H., Han S.-M., Lee D.-M. The antiallodynic effects of intrathecal cholinesterase inhibitors in a rat model of neuropathic pain. Anesthesiology 90:492-494, 1999; Lee E. J., Sim J. Y, Park J. Y., Hwang J. H., Park P. H., Han S. M. Intrathecal carbachol and clonidine produce a synergistic antiallodynic effect in rats with a nerve ligation injury. Can J Anaesth 49:178-84, 2002.). Thus, direct or indirect activation of muscarinic receptors has been shown to elicit both acute analgesic activity and to ameliorate neuropathic pain. Muscarinic agonists and ACHE-Is are not widely used clinically owing to their propensity to induced a plethora of adverse events when administered to humans. The undesirable side effects include excessive salivation and sweating, enhanced gastrointestinal motility, and bradycardia among other adverse events. These side effects are associated with the ubiquitous expression of the muscarinic family of receptors throughout the body.

To date, five subtypes of muscarinic receptors (M1-M5) have been cloned and sequenced from a variety of species, with differential distributions in the body. Therefore, it was desirable to provide molecules would permit selective modulation, for example, of muscarinic receptors controlling central nervous function without also activating muscarinic receptors controlling cardiac, gastrointestinal or glandular functions.

There is also a need for methods for treating muscarinic receptor-mediated diseases.

There is also a need for modulators of muscarinic receptors that are selective as to subtypes M1-M5.

### DESCRIPTION OF THE EMBODIMENTS

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring and 1,2,3,4-tetrahydro-naphthalene is an example of a 10-membered cycloalkyl group.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the phrase “optionally substituted” means unsubstituted or substituted. As used herein, the term “substituted” means that a hydrogen atom is removed and replaced by a substitutent. As used herein, the phrase “substituted by oxo” means that two hydrogen atoms are removed from a carbon atom and replaced by an oxygen bound by a double bond to the carbon atom. It is understood that the number of substituents for a given atom is limited by its valency.

Throughout the definitions, the term “Cₙ₋ₘ” is referred to indicate C₁₋₄, C₁₋₆, and the like, wherein n and m are integers and indicate the number of carbons, wherein n-m indicates a range which includes the endpoints.

As used herein, the term “Cₙ₋ₘ alkyl”, employed alone or in combination with other terms, refers to a saturated hydrocarbon group that may be straight-chain or branched, having n to m carbons. In some embodiments, the alkyl group contains from 1 to 7 carbon atoms, from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, n-heptyl, n-octyl, and the like.

As used herein, the term “alkylene” refers to a divalent alkyl linking group. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like.

As used herein, “Cₙ₋ₘ alkenyl”, employed alone or in combination with other terms, refers to an alkyl group having one or more double carbon-carbon bonds and having n to m carbons. In some embodiments, the alkynyl moiety contains 2 to 6 or to 2 to 5 carbon atoms. Example alkenyl groups include, but are not limited to, ethenyl, n-propenyl, isopropenyl, n-butenyl, sec-butenyl, and the like.

As used herein, the term “alkenylene”, employed alone or in combination with other terms, refers to a divalent alkenyl group. Example alkenylene groups include, but are not limited to, ethen-1,2-diyl, propen-1,3-diyl, propen-1,2-diyl, buten-1,4-diyl, buten-1,3-diyl, buten-1,2-diyl, 2-methyl-propen-1,3-diyl, and the like.

As used herein, “Cₙ₋ₘ alkynyl”, employed alone or in combination with other terms, refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6 or 2 to 5 carbon atoms.

As used herein, the term “alkynylene”, employed alone or in combination with other terms, refers to a divalent alkynyl group. In some embodiments, the alkynylene moiety contains 2 to 12 carbon atoms. In some embodiments, the alkynylene moiety contains 2 to 6 carbon atoms. Example alkynylene groups include, but are not limited to, ethyn-1,2-diyl, propyn-1,3,-diyl, 1-butyn-1,4-diyl, 1-butyn-1,3-diyl, 2-butyn-1,4-diyl, and the like.

As used herein, the term “Cₙ₋ₘ alkoxy”, employed alone or in combination with other terms, refers to an group of formula —O-alkyl, wherein the alkyl group has n to m carbons. Example alkoxy groups include methoxy, ethoxy, propoxy (e.g., n-propoxy and isopropoxy), t-butoxy, and the like.

As used herein, the term “Cₙ₋ₘ aryl”, employed alone or in combination with other terms, refers to a monocyclic or polycyclic (e.g., having 2, 3 or 4 fused or covalently linked rings), aromatic hydrocarbon having n to m carbons, such as, but not limited to, phenyl, 1-naphthyl, 2-naphthyl, anthracenyl, phenanthrenyl, and the like. In some embodiments, aryl groups have from 6 to 20 carbon atoms, from 6 to 10 carbon atoms, or from 6 to 8 carbons atoms. In some embodiments, the aryl group is phenyl.

As used herein, the term “Cₙ₋ₘ aryl-Cₙ₋ₘalkyl” refers to a group of formula -alkylene-aryl, wherein the alkyl and aryl portions each has, independently, n to m carbon atoms. In some embodiments, the alkyl portion has 1 to 4, 1 to 3, 1 to 2, or 1 carbon atom(s). In some embodiments, the alkyl portion of the arylalkyl group is methyl or ethyl. In some embodiments, the arylalkyl group is benzyl.

As used herein, the term “Cₙ₋ₘ cycloalkyl”, employed alone or in combination with other terms, refers to a non-aromatic cyclic hydrocarbon moiety, which may optionally contain one or more alkenylene or alkynylene groups as part of the ring structure and which has n to m carbons. Cycloalkyl groups can include mono- or polycyclic (e.g., having 2, 3 or 4 fused or covalently linked rings) ring systems. Also included in the definition of cycloalkyl are moieties that have one or more aromatic rings fused (i.e., having a bond in common with) to the cycloalkyl ring, for example, benzo derivatives of pentane, pentene, hexane, and the like. In some embodiments, the cycloalkyl group is monocyclic and has 3 to 14 ring members, 3 to 10 ring members, 3 to 8 ring members, or 3 to 7 ring members. One or more ring-forming carbon atoms of a cycloalkyl group can be oxidized to form carbonyl linkages. Exemplary cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, the cycloalkyl group is cyclopropyl, cyclobutyl, cyclopentyl, or cyclohexyl.

As used herein, the term “Cₙ₋ₘ cycloalkyl-Cₙ₋ₘalkyl” refers to a group of formula -alkylene-cycloalkyl, wherein the alkyl and cycloalkyl portions each has, independently n to m carbon atoms. In some embodiments, the alkyl portion has 1 to 4, 1 to 3, 1 to 2, or 1 carbon atom(s).

As used herein, “Cₙ₋ₘ haloalkoxy”, employed alone or in combination with other terms, refers to a group of formula —O-haloalkyl having n to m carbon atoms. An example haloalkoxy group is OCF₃. In some embodiments, the haloalkoxy group is fluorinated only.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only.

As used herein, the term “fluorinated Cₙ₋ₘ haloalkyl” refers to a Cₙ₋ₘ haloalkyl wherein the halogen atoms are selected from fluorine. In some embodiments, fluorinated Cₙ₋ₘ haloalkyl is fluoromethyl, difluoromethyl, or trifluoromethyl.

As used herein, the terms “halo” and “halogen”, employed alone or in combination with other terms, refer to fluoro, chloro, bromo, and iodo. In some embodiments, halogen is fluoro, bromo, or chloro. In some embodiments, halogen is fluoro or chloro.

As used herein, the term “Cₙ₋ₘ heteroaryl”, “Cₙ₋ₘ heteroaryl ring”, or “Cₙ₋ₘ heteroaryl group”, employed alone or in combination with other terms, refers to a monocyclic or polycyclic (e.g., having 2, 3 or 4 fused or covalently linked rings) aromatic hydrocarbon moiety, having one or more heteroatom ring members selected from nitrogen, sulfur and oxygen, and having n to m carbon atoms. In some embodiments, the heteroaryl group has 1, 2, 3, or 4 heteroatoms. In some embodiments, the heteroaryl group has 1, 2, or 3 heteroatoms. In some embodiments, the heteroaryl group has 1 or 2 heteroatoms. In some embodiments, the heteroaryl group has 1 heteroatom. When the heteroaryl group contains more than one heteroatom ring member, the heteroatoms may be the same or different. Example heteroaryl groups include, but are not limited to, pyrrolyl, azolyl, oxazolyl, thiazolyl, imidazolyl, furyl, thienyl, quinolinyl, isoquinolinyl, indolyl, benzothienyl, benzofuranyl, benzisoxazolyl, imidazo[1,2-b]thiazolyl or the like. In some embodiments, the heteroaryl group has 5 to 10 carbon atoms.

As used herein, the term “Cₙ₋ₘ heteroaryl-Cₙ₋ₘalkyl” refers to a group of formula -alkylene-heteroaryl, wherein the alkyl and heteroaryl portions each has, independently, n to m carbon atoms. In some embodiments, the alkyl portion has 1 to 4, 1 to 3, 1 to 2, or 1 carbon atom(s).

As used herein, the term “Cₙ₋ₘ heterocycloalkyl”, “Cₙ₋ₘ heterocycloalkyl ring”, or “Cₙ₋ₘ heterocycloalkyl group”, employed alone or in combination with other terms, refers to non-aromatic ring system, which may optionally contain one or more alkenylene or alkynylene groups as part of the ring structure, and which has at least one heteroatom ring member selected from nitrogen, sulfur and oxygen, and which has n to m carbon atoms. In some embodiments, the heteroaryl group has 1, 2, 3, or 4 heteroatoms. In some embodiments, the heteroaryl group has 1, 2, or 3 heteroatoms. In some embodiments, the heteroaryl group has 1 or 2 heteroatoms. In some embodiments, the heteroaryl group has 1 heteroatom. In some embodiments, the heteroaryl group has 1 or 2 heteroatoms. When the heterocycloalkyl groups contains more than one heteroatom, the heteroatoms may be the same or different. Heterocycloalkyl groups can include mono- or polycyclic (e.g., having 2, 3 or 4 fused or covalently bonded rings) ring systems. Also included in the definition of heterocycloalkyl are moieties that have one or more aromatic rings fused (i.e., having a bond in common with) to the non-aromatic ring, for example, 1,2,3,4-tetrahydro-quinoline and the like. In some embodiments, the heterocycloalkyl group has 3 to 20 ring-forming atoms, 3 to 10 ring-forming atoms, or about 3 to 8 ring forming atoms. The carbon atoms or hetereoatoms in the ring(s) of the heterocycloalkyl group can be oxidized to form a carbonyl, or sulfonyl group (or other oxidized linkage) or a nitrogen atom can be quaternized. In some embodiments, the heterocycloalkyl group is a monocyclic or bicyclic ring. In some embodiments, the heterocycloalkyl group is a monocyclic ring, wherein the ring comprises from 3 to 6 carbon atoms and from 1 to 3 heteroatoms, referred to herein as C₃₋₆heterocycloalkyl.

Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl.

A five-membered ring heteroaryl is a heteroaryl with a ring having five ring atoms wherein 1, 2 or 3 ring atoms are independently selected from N, O, and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O, and S. Exemplary six-membered ring heteroaryls are pyridyl, pyrazinyl, pyrimidinyl, triazinyl and pyridazinyl.

As used herein, the term “Cₙ₋ₘ heterocycloalkyl-Cₙ₋ₘalkyl” refers to a group of formula -alkylene-heterocycloalkyl, wherein the alkyl and heterocycloalkyl portions each has, independently, n to m carbon atoms. In some embodiments, the alkyl portion of the heterocycloalkylalkyl group is methylene. In some embodiments, the alkyl portion has 1-4, 1-3, 1-2, or 1 carbon atom(s).

As used herein, the moiety “C(O)” indicates a divalent carbonyl group of formula C(═O).

As used herein, the term “—C(O)OR^{a}” refers to a group of formula —C(═O)OR^{a}, linked at the carbonyl group.

As used herein, the term “—CO₂R^{e}” refers to a group of formula —C(═O)OR^{e}, linked at the carbonyl group.

As used herein, the term “—C(O)R^{b}” refers to a group of formula —C(═O)R^{b}, linked at the carbonyl group.

As used herein, the term “—C(O)—R^{e}” refers to a group of formula —C(═O)R^{e}, linked at the carbonyl group.

As used herein, the term “—C(O)NR^{c}R^{d}—” refers to a group of formula —C(═O)NR^{c}R^{d}, linked at the carbonyl group.

As used herein, the term “—C(O)—NR^{e}R^{f}” refers to a group of formula —C(═O)—NR^{e}R^{f}, linked at the carbonyl group.

As used herein, the term “—SO₂R^{e}” refers to a group of formula —S(═O)₂R^{e}, linked at the sulfur atom of the sulfonyl group.

As used herein, the term “—SO₂NR^{e}R^{f}” refers to a group of formula —S(═O)₂NR^{e}R^{f}, linked at the sulfur atom of the sulfonyl group.

In general, a hyphen in a formula at the beginning of a substituent indicates the point of attachment. For example, in the term “—SO₂R^{e}”, the hyphen indicates that the point of attachment is the sulfur atom.

### Compounds

In one aspect, the present invention provides a compound of Formula I:

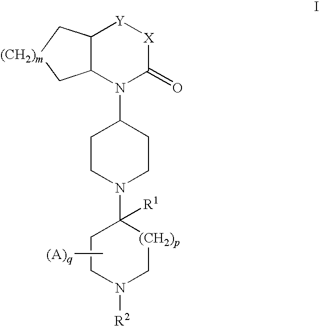

or pharmaceutically acceptable salt thereof;
wherein:

Y is —CR³R⁴—, —NR⁵—, —O—, or —S—;

X is —CR⁶R⁷—, —NR⁸—, —O—, or —S—;

with the proviso that either Y is —CR³R⁴— or X is —CR⁶R⁷—;

each A is, independently, C₁₋₃ alkyl, or two A linked together to form a C₁₋₃alkylene bridge;

R¹ is hydrogen, C₁₋₆ alkyl, or C₁₋₆ haloalkyl;

R² is —C(O)OR^{a}, —C(O)R^{b}, —C(O)NR^{c}R^{d}, C₁₋₆ alkyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀-aryl-C₁₋₃-alkyl, C₃₋₉ heteroaryl, or C₃₋₉heteroaryl-C₁₋₃alkyl; wherein said C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, 3, or 4 independently selected R⁹ groups; wherein said C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, 3, or 4 independently selected R¹⁰ groups; and wherein the C₁₋₆ alkyl, C₂₋₆ alkenyl, C₂₋₆ alkynyl C₁₋₆ haloalkyl, C₁₋₆ alkoxy, and C₁₋₆ haloalkoxy are each optionally substituted by 1, 2, or 3 independently selected R¹¹ groups;

R³, R⁴, R⁶, and R⁷ are each, independently, hydrogen, fluoro, C₁₋₄ alkyl, C₁₋₄ alkoxymethyl, cyanoC₁₋₄ alkyl or C₁₋₄ haloalkyl; R⁵ and R⁸ are each, independently, hydrogen, C₁₋₄ alkyl, or C₁₋₄ haloalkyl;

each R⁹ and R¹⁰ is, independently, phenyl, C₃₋₆ cycloalkyl, C₂₋₅ heterocycloalkyl, C₃₋₅ heteroaryl, —CN, —SR^{e}, —OR^{e}, —O(CH₂)ᵣ—OR^{e}, R^{e}, —C(O)—R^{e}, —CO₂R^{e}, —SO₂R^{e}, —SO₂NR^{e}R^{f}, halogen, —NO₂, —NR^{e}R^{f}, —(CH₂)ᵣNR^{e}R^{f}, or —C(O)—NR^{e}R^{f};

each R¹¹ is, independently, —CN, —NO₂, —OR^{e}, or —NR^{e}R^{f};

R^{a}, R^{b}, R^{c}, and R^{d} are each, independently, hydrogen, C₁₋₇ alkyl, C₂₋₆ alkenyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein said C₆₋₁₀aryl, C₆₋₁₀aryl-C₁₋₃alkyl, C₃₋₉ heteroaryl, and C₃₋₉ heteroaryl-C₁₋₃alkyl are each optionally substituted by 1, 2, 3, or 4 independently selected R¹² groups; wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl, are each optionally substituted by 1, 2, 3, or 4 independently selected R¹³ groups; and wherein the C₁₋₇ alkyl, C₂₋₆ alkenyl, C₂₋₆ alkynyl, C₁₋₆ haloalkyl, C₁₋₇ alkoxy, and C₁₋₆ haloalkoxy are each optionally substituted by 1, 2, or 3 independently selected R¹⁴ groups;

each R¹², R¹³, and R¹⁴ is, independently, phenyl, C₃₋₆ cycloalkyl, C₂₋₅ heterocycloalkyl, C₃₋₅ heteroaryl, —CN, —SR^{g}, —OR^{g}, —O(CH₂)ᵣ—OR^{g}, R^{g}, —C(O)—R^{g}, —CO₂R^{g}, —SO₂R^{g}, —SO₂NR^{g}R^{h}, halogen, —NO₂, —NR^{g}R^{h}, —(CH₂)ᵣNR^{g}R^{h}, or —C(O)—NR^{g}R^{h};

each R^{e}, R^{f}, R^{g} and R^{h} is, independently hydrogen, C₁₋₆ alkyl, C₂₋₆ alkenyl or C₁₋₆ haloalkyl,

m is 1, 2, or 3;

p is 0, 1, or2;

q is an integer from 0 to [6+(p×2)]; and

r is 1, 2, 3 or 4;

with the proviso that the compound is not isopropyl 4′-methyl-4-((4aS,8aS)-2-oxooctahydroquinoxalin-1(2H)-yl)-1,4′-bipiperidine-1′-carboxylate, or pharmaceutically acceptable salt thereof.

In some embodiments:

Y is —CR³R⁴—, —NR⁵—, or —O—; and

X is —CR⁶R⁷—, —NR⁸—, or —O—.

In some embodiments:

Y is —CR³R⁴— or —O—; and

X is —CR⁶R⁷—, —NR⁸—, or —O—.

In some embodiments, Y is —CR³R⁴—. In some embodiments, Y is —NR⁵—. In some embodiments, Y is —O—. In some embodiments, Y is —S—.

In some embodiments, X is —CR⁶R⁷—. In some embodiments, X is —NR⁸—. In some embodiments, X is —O—. In some embodiments, X is —S—.

In some embodiments, X is not —S—

In some embodiments, Y is not —S—.

In some embodiments, when Y is —CR³R⁴—, then X is not —CR⁶R⁷—; and when X is —CR⁶R⁷—, then Y is not —CR³R⁴—.

In some embodiments, when X is —CR⁶R⁷—, then Y is not —CR³R⁴— or —NR⁵—; and when Y is —CR³R⁴—, then X is not —CR⁶R⁷—.

In some embodiments, X is not —S—; Y is not —S—; when X is —CR⁶R⁷—, then Y is not —CR³R⁴— or —NR⁵—; and when Y is —CR³R⁴—, then X is not —CR⁶R⁷—.

In some embodiments, X is not —S—; Y is not —S—; when X is —CR⁶R⁷—, then Y is not —CR³R⁴—; and when Y is —CR³R⁴—, then X is not —CR⁶R⁷—.

In some embodiments, R¹ is hydrogen or C₁₋₆ alkyl.

In some embodiments, R¹ is hydrogen, C₁₋₆ alkyl, or fluorinated C₁₋₆ haloalkyl.

In some embodiments, R¹ is hydrogen or C₁₋₄ alkyl.

In some embodiments, R¹ is hydrogen, C₁₋₄ alkyl, or fluorinated C₁₋₄ haloalkyl

In some embodiments, R¹ is hydrogen or C₁₋₃ alkyl.

In some embodiments, R¹ is hydrogen, C₁₋₃ alkyl, or fluorinated C₁₋₃ haloalkyl

In some embodiments, R¹ is hydrogen or methyl.

In some embodiments, R¹ is hydrogen, methyl, or fluorinated methyl.

In some embodiments, R¹ is hydrogen, C₁₋₃ alkyl, fluoromethyl, difluoromethyl, or trifluoromethyl.

In some embodiments, R¹ is hydrogen, methyl, ethyl, fluoromethyl, difluoromethyl, or trifluoromethyl.

In some embodiments, R² is —C(O)OR^{a}, —C(O)R^{b}, —C(O)NR^{c}R^{d}, C₃₋₇ cycloalkyl-C₁₋₃alkyl, C₃₋₇ heterocycloalkyl-C₁₋₃ alkyl, C₆₋₁₀ aryl-C₁₋₃alkyl, or C₃₋₉ heteroaryl-C₁₋₃alkyl; wherein the C₆₋₁₀ aryl-C₁₋₃alkyl and C₃₋₉ heteroaryl-C₁₋₃alkyl are each optionally substituted by 1, 2, 3, or 4 independently selected R⁹ groups; and wherein the C₃₋₇ cycloalkyl-C₁₋₃alkyl and C₃₋₇ heterocycloalkyl-C₁₋₃ alkyl are each optionally substituted by 1, 2, 3, or 4 independently selected R¹⁰ groups.

In some embodiments, R² is —C(O)OR^{a}, —C(O)R^{b}, —C(O)NR^{c}R^{d}, —CH₂—C₃₋₇ cycloalkyl, —CH₂—C₃₋₇ heterocycloalkyl, —CH₂—C₆₋₁₀aryl, or —CH₂—C₆₋₉heteroaryl; wherein said —CH₂—C₆₋₁₀aryl and —CH₂—C₆₋₉heteroaryl are each optionally substituted by 1, 2, 3, or 4 independently selected R⁹ groups; and wherein said —CH₂—C₃₋₇ cycloalkyl and —CH₂—C₃₋₇ heterocycloalkyl, are each optionally substituted by 1, 2, 3, or 4 independently selected R¹⁰ groups.

In some embodiments, R² is —C(O)OR^{a}, —C(O)R^{b}, —C(O)NR^{c}R^{d}, C₆₋₁₀aryl-C₁₋₃alkyl or C₃₋₉heteroaryl-C₁₋₃alkyl; wherein said C₆₋₁₀aryl-C₁₋₃alkyl and C₃₋₉heteroaryl-C₁₋₃alkyl are each optionally substituted by 1, 2, or 3 independently selected R⁹ groups.

In some embodiments, R² is —C(O)OR^{a}, —C(O)R^{b}, —C(O)NR^{c}R^{d}, —CH₂—C₆₋₁₀aryl, or —CH₂—C₆₋₉heteroaryl; wherein said —CH₂—C₆₋₁₀aryl and —CH₂—C₆₋₉heteroaryl are each optionally substituted by 1, 2, 3, or 4 independently selected R⁹ groups.

In some embodiments, R² is —C(O)OR^{a}, —C(O)R^{b}, or —C(O)NR^{c}R^{d}.

In some embodiments, R² is —C(O)OR^{a} or —C(O)R^{b}.

In some embodiments, R³, R⁴, R⁶, and R⁷ are each, independently, hydrogen or C₁₋₄ alkyl.

In some embodiments, R³, R⁴, R⁶, and R⁷ are hydrogen.

In some embodiments, R⁵ and R⁸ are each, independently, hydrogen or C₁₋₄ alkyl.

In some embodiments, R⁵ and R⁸ are each, independently, hydrogen or methyl.

In some embodiments, R⁵ and R⁸ are each, independently, hydrogen.

In some embodiments, R⁵ and R⁸ are each, independently, C₁₋₄ alkyl.

In some embodiments, R⁵ is, independently, hydrogen.

In some embodiments, R⁵ is, independently, C₁₋₄ alkyl.

In some embodiments, R⁸ is, independently, hydrogen.

In some embodiments, R⁸ is, independently, C₁₋₄ alkyl.

In some embodiments, R⁵ and R⁸ are each, independently, C₁₋₄ alkyl.

In some embodiments, R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀ aryl, C₆₋₁₀ aryl-C₁₋₃ alkyl, C₃₋₉ heteroaryl, or C₃₋₉ heteroaryl-C₁₋₃alkyl; wherein said C₆₋₁₀ aryl, C₆₋₁₀ aryl-C₁₋₃alkyl, C₃₋₉ heteroaryl, and C₃₋₉ heteroaryl-C₁₋₃alkyl are each optionally substituted with 1, 2, or 3 independently selected R¹² groups; and wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl are each optionally substituted with 1, 2, or 3 independently selected R¹³ groups.

In some embodiments, R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₆₋₁₀ aryl, or C₃₋₉ heteroaryl; wherein said C₆₋₁₀ aryl and C₃₋₉ heteroaryl are each optionally substituted with 1, 2, or 3 independently selected R¹² groups.

In some embodiments, R^{a}, R^{b}, R^{c}, and R^{d} are each, independently, C₁₋₇ alkyl, —CH₂—(C₂₋₅ alkynyl), C₁₋₆ haloalkyl, C₃₋₇ cycloalkyl, C₆₋₁₀ aryl, or C₃₋₉ heteroaryl; wherein said C₆₋₁₀ aryl and C₃₋₉ heteroaryl are each optionally substituted with 1, 2, or 3 independently selected R¹² groups.

In some embodiments, R^{a}, R^{b}, R^{c}, and R^{d} are each, independently, C₁₋₇ alkyl, C₁₋₆ haloalkyl, C₃₋₇ cycloalkyl, phenyl, or C₃₋₇ heteroaryl; wherein said phenyl or C₃₋₉ heteroaryl is each optionally substituted with 1 or 2 independently selected R¹² groups.

In some embodiments, R^{a} and R^{b} are each, independently, C₁₋₇ alkyl, C₁₋₆ haloalkyl, C₃₋₇ cycloalkyl, phenyl, or C₃₋₉ heteroaryl; wherein said phenyl or C₃₋₉ heteroaryl is each optionally substituted with 1 or 2 independently selected R¹² groups.

In some embodiments, R^{a} is, independently, ethyl, isopropyl, or cyclopropyl.

In some embodiments, R^{b} is, independently, phenyl, pyrrolyl, or thienyl, wherein the phenyl, pyrrolyl or thienyl is optionally substituted with 1 R¹² group.

In some embodiments, R^{a} is, independently, ethyl, isopropyl, or cyclopropyl; and R^{b} is, independently, phenyl, pyrrolyl, or thienyl, wherein the phenyl, pyrrolyl or thienyl is optionally substituted with 1 R¹² group.

In some embodiments, each R¹² is, independently, halogen, —CN, —NO₂, —OH, C₁₋₆ alkyl, C₁₋₆ haloalkyl, C₁₋₆ alkoxy, C₁₋₆ haloalkoxy, —NR^{g}R^{h}, —(CH₂)ᵣNR^{g}R^{h} or —SO₂R^{g}.

In some embodiments, each R¹² is, independently, halogen, —CN, —NO₂, —OH, C₁₋₆ alkyl, C₁₋₆ haloalkyl, C₁₋₆ alkoxy, C₁₋₆ haloalkoxy, or —NR^{g}R^{h}.

In some embodiments, each R¹² is, independently, C₁₋₆ alkyl, C₁₋₆ haloalkyl, C₁₋₆ alkoxy, or C₁₋₆ haloalkoxy.

In some embodiments, each R¹² is, independently, C₁₋₆ alkyl or C₁₋₆ alkoxy.

In some embodiments, each R¹² is, independently, methoxy or methyl.

In some embodiments, each R¹³ is, independently, C₁₋₆ alkyl, C₁₋₆ haloalkyl, C₁₋₆ alkoxy, or C₁₋₆ haloalkoxy.

In some embodiments, each R¹⁴ is, independently, C₁₋₆ alkyl, C₁₋₆ haloalkyl, C₁₋₆ alkoxy, or C₁₋₆ haloalkoxy.

In some embodiments, each R⁹ is, independently, halogen, —CN, —NO₂, hydroxyl, C₁₋₆ alkyl, C₁₋₆ haloalkyl, C₁₋₆ alkoxy, C₁₋₆ haloalkoxy, —NR^{e}R^{f}, —(CH₂)ᵣNR^{e}R^{f} or —SO₂R^{e}.

In some embodiments, each R⁹ is, independently, halogen, —CN, —NO₂, —OH, C₁₋₆ alkyl, C₁₋₆ haloalkyl, C₁₋₆ alkoxy, or C₁₋₆ haloalkoxy.

In some embodiments, each R¹⁰ is, independently, —OH, —CN, —NO₂, hydroxyl, C₁₋₆ alkyl, C₁₋₆ haloalkyl, C₁₋₆ alkoxy, C₁₋₆ haloalkoxy, —NR^{e}R^{f}, —(CH₂)ᵣNR^{e}R^{f} or —SO₂R^{e}.

In some embodiments, each R¹⁰ is, independently, C₁₋₄ alkyl, C₁₋₄ haloalkyl, C₁₋₄ alkoxy, or C₁₋₄ haloalkoxy.

In some embodiments, m is 2.

In some embodiments, p is 0 or 1.

In some embodiments, each A is methyl.

In some embodiments, q is 1, 2, 3, or 4. In some embodiments, q is 1, 2, or 3. In some embodiments, q is 1 or 2. In some embodiments, q is 1. In some embodiments, q is 0.

In some embodiments, each R^{e}, R^{f}, R^{g} and R^{h} is, independently hydrogen, C₁₋₆ alkyl, or C₂₋₆ or C₁₋₆ haloalkyl.

In some embodiments, r is 1, 2, or 3.

In some embodiments, r is 1 or 2.

In some embodiments, r is 1.

In some embodiments:
R^{a} is, independently, ethyl, isopropyl, or cyclopropyl; and
R^{b} is, independently, 2-methylphenyl, N-methylpyrrol-2-yl, or 3-methoxythien-2-yl.

In some embodiments, each R^{e}, R^{f}, R^{g} and R^{h} is, independently hydrogen or C₁₋₆ alkyl.

In some embodiments:
Y is —CR³R⁴—, —NR⁵—, or —O—;
X is —CR⁶R⁷—, —NR⁸—, or —O—;
with the proviso that either Y is —CR³R⁴— or X is —CR⁶R⁷—;
R¹ is hydrogen or C₁₋₆ alkyl;
R² is —C(O)OR^{a}, —C(O)R^{b}, —C(O)NR^{c}R^{d}, C₃₋₇ cycloalkyl-C₁₋₃alkyl, C₃₋₇ heterocycloalkyl-C₁₋₃ alkyl, C₆₋₁₀ aryl-C₁₋₃alkyl, or C₃₋₉ heteroaryl-C₁₋₃alkyl; wherein the C₆₋₁₀ aryl-C₁₋₃alkyl and C₃₋₉ heteroaryl-C₁₋₃alkyl are each optionally substituted by 1, 2, 3, or 4 independently selected R⁹ groups; and wherein the C₃₋₇ cycloalkyl-C₁₋₃alkyl and C₃₋₇ heterocycloalkyl-C₁₋₃ alkyl are each optionally substituted by 1, 2, 3, or 4 independently selected R¹⁰ groups;
R³, R⁴, R⁶, and R⁷ are each, independently, hydrogen or C₁₋₄ alkyl;
R⁵ and R⁸ are each, independently, hydrogen or C₁₋₄ alkyl;
each R⁹ is, independently, halogen, —CN, —NO₂, —OH, C₁₋₆ alkyl, C₁₋₆ haloalkyl, C₁₋₆ alkoxy, C₁₋₆ haloalkoxy. —NR^{e}R^{f}, —(CH₂)ᵣNR^{e}R^{f} or —SO₂R^{e};
each R¹⁰ is, independently, —CN, —NO₂, —OH, C₁₋₆ alkyl, C₁₋₆ haloalkyl, C₁₋₆ alkoxy, C₁₋₆ haloalkoxy, —NR^{e}R^{f}, —(CH₂)ᵣNR^{e}R^{f} or —SO₂R^{e};
R^{a}, R^{b}, R^{c}, and R^{d} are each, independently, hydrogen, C₁₋₇ alkyl, C₂₋₆ alkenyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein said C₆₋₁₀aryl, C₆₋₁₀aryl-C₁₋₃alkyl, C₃₋₉ heteroaryl, and C₃₋₉ heteroaryl-C₁₋₃alkyl are each optionally substituted by 1, 2, 3, or 4 independently selected R¹² groups; wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl, are each optionally substituted by 1, 2, 3, or 4 independently selected R¹³ groups; and wherein the C₁₋₇ alkyl, C₂₋₆ alkenyl, C₂₋₆ alkynyl, C₁₋₆ haloalkyl, C₁₋₇ alkoxy, and C₁₋₆ haloalkoxy are each optionally substituted by 1, 2, or 3 independently selected R¹⁴ groups;
each R¹² is, independently, halogen, —CN, —NO₂, hydroxyl, C₁₋₆ alkyl, C₁₋₆ haloalkyl, C₁₋₆ alkoxy, C₁₋₆ haloalkoxy, —NR^{g}R^{h}, —(CH₂)ᵣNR^{g}R^{h} or —SO₂R^{g};
each R¹³ is, independently, —CN, —NO₂, —OH, C₁₋₆ alkyl, C₁₋₆ haloalkyl, C₁₋₆ alkoxy, C₁₋₆ haloalkoxy, —NR^{g}R^{h}, —(CH₂)ᵣNR^{g}R^{h} or —SO₂R^{g}.
each R¹⁴ is, independently, —CN, —NO₂, —OH, C₁₋₆ alkoxy, C₁₋₆ haloalkoxy, —NR^{g}R^{h}, —(CH₂)ᵣNR^{g}R^{h} or —SO₂R^{g}; and
each R^{e}, R^{f}, R^{g} and R^{h} is, independently hydrogen or C₁₋₆ alkyl;
   or pharmaceutically acceptable salt thereof.

In some embodiments:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or C₁₋₆ alkyl;
R² is —C(O)OR^{a}, —C(O)R^{b}, —C(O)NR^{c}R^{d}, C₃₋₇ cycloalkyl-C₁₋₃alkyl, C₃₋₇ heterocycloalkyl-C₁₋₃ alkyl, C₆₋₁₀ aryl-C₁₋₃alkyl, or C₃₋₉ heteroaryl-C₁₋₃alkyl; wherein the C₆₋₁₀ aryl-C₁₋₃alkyl and C₃₋₉ heteroaryl-C₁₋₃alkyl are each optionally substituted by 1, 2, 3, or 4 independently selected R⁹ groups; and wherein the C₃₋₇ cycloalkyl-C₁₋₃alkyl and C₃₋₇ heterocycloalkyl-C₁₋₃ alkyl are each optionally substituted by 1, 2, 3, or 4 independently selected R¹⁰ groups;
R³, R⁴, R⁶, and R⁷ are each hydrogen;
R⁸ is, independently, hydrogen or C₁₋₄ alkyl;
each R⁹ is, independently, halogen, —CN, —NO₂, —OH, C₁₋₄ alkyl, C₁₋₄ haloalkyl, C₁₋₄ alkoxy, or C₁₋₄ haloalkoxy;
each R¹⁰ is, independently, C₁₋₄ alkyl, C₁₋₄ haloalkyl, C₁₋₄ alkoxy, or C₁₋₄ haloalkoxy;
R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₃₋₇cycloalkyl-C₁₋₃alkyl, C₃₋₇ heterocycloalkyl, C₃₋₇ heterocycloalkyl-C₁₋₃ alkyl, C₆₋₁₀ aryl, C₆₋₁₀ aryl-C₁₋₃ alkyl, C₃₋₉ heteroaryl, or C₃₋₉ heteroaryl-C₁₋₃alkyl; wherein said C₆₋₁₀ aryl, C₆₋₁₀ aryl-C₁₋₃alkyl, C₃₋₉ heteroaryl, and C₃₋₉ heteroaryl-C₁₋₃alkyl where each optionally substituted with 1, 2, or 3 independently selected R¹² groups; and wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl are each optionally substituted with 1, 2, or 3 independently selected R¹³ groups.
each R¹² is, independently, halogen, —CN, —NO₂, —OH, C₁₋₆ alkyl, C₁₋₆ haloalkyl, C₁₋₆ alkoxy, C₁₋₆ haloalkoxy, or —NR^{g}R^{h};
each R¹³ is, independently, C₁₋₆ alkyl, C₁₋₆ haloalkyl, C₁₋₆ alkoxy, or C₁₋₆ haloalkoxy; and
each R^{g} and R^{h} is, independently hydrogen or C₁₋₆ alkyl.

In some embodiments:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or C₁₋₆ alkyl;
R² is —C(O)OR^{a}, —C(O)R^{b}, —C(O)NR^{c}R^{d}, —CH₂-cycloalkyl, —CH₂-heterocycloalkyl, —CH₂-aryl, or —CH₂-heteroaryl; wherein said —CH₂-aryl and —CH₂-heteroaryl are each optionally substituted by 1, 2, 3, or 4 independently selected R⁹ groups;
R³, R⁴, R⁶, and R⁷ are each hydrogen;
R⁸ is, independently, hydrogen or C₁₋₄ alkyl;
each R⁹ is, independently, halogen, —CN, —NO₂, —OH, C₁₋₄ alkyl, C₁₋₄ haloalkyl, C₁₋₄ alkoxy, or C₁₋₄ haloalkoxy;
each R¹⁰ is, independently, C₁₋₄ alkyl, C₁₋₄ haloalkyl, C₁₋₄ alkoxy, or C₁₋₄ haloalkoxy;
R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀ aryl, C₆₋₁₀ aryl-C₁₋₃ alkyl, C₃₋₉ heteroaryl, or C₃₋₉ heteroaryl-C₁₋₃alkyl; wherein said C₆₋₁₀ aryl, C₆₋₁₀ aryl-C₁₋₃alkyl, C₃₋₉ heteroaryl, and C₃₋₉ heteroaryl-C₁₋₃alkyl are each optionally substituted with 1, 2, or 3 independently selected R¹² groups; and wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl are each optionally substituted with 1, 2, or 3 independently selected R¹³ groups;
each R¹² is, independently, halogen, CN, —NO₂, —OH, C₁₋₆ alkyl, C₁₋₆ haloalkyl, C₁₋₆ alkoxy, C₁₋₆ haloalkoxy, or —NR^{g}R^{h}; and
each R¹³ is, independently, C₁₋₆ alkyl, C₁₋₆ haloalkyl, C₁₋₆ alkoxy, or C₁₋₆ haloalkoxy; and
each R^{g} and R^{h} is, independently hydrogen or C₁₋₆ alkyl.

In some embodiments:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or C₁₋₃ alkyl;
R² is —C(O)OR^{a}, —C(O)R^{b}, —C(O)NR^{c}R^{d}, C₆₋₁₀aryl-C₁₋₃alkyl or C₃₋₉heteroaryl-C₁₋₃alkyl; wherein said C₆₋₁₀aryl-C₁₋₃alkyl and C₃₋₉heteroaryl-C₁₋₃alkyl are each optionally substituted by 1, 2, or 3 independently selected R⁹ groups;
R³, R⁴, R⁶, and R⁷ are each hydrogen;
R⁸ is, independently, hydrogen or C₁₋₃ alkyl;
each R⁹ is, independently, C₁₋₄ alkyl, C₁₋₄ haloalkyl, C₁₋₄ alkoxy, and C₁₋₄ haloalkyl;
R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₆₋₁₀ aryl, or C₃₋₉ heteroaryl; wherein said C₆₋₁₀ aryl and C₃₋₉ heteroaryl are each optionally substituted with 1, 2, or 3 independently selected R¹² groups; and
each R¹² is, independently, C₁₋₆ alkyl, C₁₋₆ haloalkyl, C₁₋₆ alkoxy, or C₁₋₆ haloalkoxy.

In some embodiments:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or C₁₋₃ alkyl;
R² is —C(O)OR^{a}, —C(O)R^{b}, —C(O)NR^{c}R^{d}, —CH₂—C₆₋₁₀aryl, or —CH₂—C₆₋₉heteroaryl; wherein said —CH₂—C₆₋₁₀aryl and —CH₂—C₆₋₉heteroaryl are each optionally substituted by 1, 2, 3, or 4 independently selected R⁹ groups;
R³, R⁴, R⁶, and R⁷ are each hydrogen;
R⁸ is, independently, hydrogen or C₁₋₃ alkyl;
each R⁹ is, independently, C₁₋₄ alkyl, C₁₋₄ haloalkyl, C₁₋₄ alkoxy, and C₁₋₄ haloalkyl;
R^{a}, R^{b}, R^{c}, and R^{d} are each, independently, C₁₋₇ alkyl, —CH₂—(C₂₋₅ alkynyl), C₁₋₆ haloalkyl, C₃₋₇ cycloalkyl, C₆₋₁₀ aryl, or C₃₋₉ heteroaryl; wherein said C₆₋₁₀ aryl and C₃₋₉ heteroaryl are each optionally substituted with 1, 2, or 3 independently selected R¹² groups; and
each R¹² is, independently, C₁₋₆ alkyl, C₁₋₆ haloalkyl, C₁₋₆ alkoxy, or C₁₋₆ haloalkoxy.

In some embodiments:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or C₁₋₃ alkyl;
R² is —C(O)OR^{a}, and —C(O)R^{b};
R³, R⁴, R⁶, and R⁷ are each hydrogen;
R⁸ is, independently, hydrogen or C₁₋₂ alkyl;
R^{a}, R^{b}, R^{c}, and R^{d} are each, independently, C₁₋₇ alkyl, C₁₋₆ haloalkyl, C₃₋₇ cycloalkyl, phenyl, or C₃₋₇ heteroaryl; wherein said phenyl or C₃₋₉ heteroaryl is each optionally substituted with 1 or 2 independently selected R¹² groups; and
each R¹² is, independently, C₁₋₆ alkyl, C₁₋₆ haloalkyl, C₁₋₆ alkoxy, or C₁₋₆ haloalkoxy.

In some embodiments:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or C₁₋₃ alkyl;
R² is —C(O)OR^{a}, and —C(O)R^{b};
R³, R⁴, R⁶, and R⁷ are each hydrogen;
R⁸ is, independently, hydrogen or C₁₋₃ alkyl;
R^{a} and R^{b} are each, independently, C₁₋₄ alkyl, C₁₋₄ haloalkyl, C₃₋₇ cycloalkyl, phenyl, or C₃₋₉ heteroaryl; wherein said phenyl or C₃₋₉ heteroaryl is each optionally substituted with 1 or 2 independently selected R¹² groups; and
each R¹² is, independently, C₁₋₆ alkyl or C₁₋₆ alkoxy.

In some embodiments:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or methyl; and
R² is —C(O)OR^{a}, and —C(O)R^{b};
R³, R⁴, R⁶, and R⁷ are each hydrogen;
R⁸ is, independently, hydrogen or methyl;
R^{a} is, independently, ethyl, isopropyl, or cyclopropyl;
R^{b} is, independently, phenyl, pyrrolyl, or thienyl, wherein the phenyl, pyrrolyl or thienyl is optionally substituted with 1 R¹² group; and
each R¹² is, independently, methoxy or methyl.

In some embodiments:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or methyl; and
R² is —C(O)OR^{a}, and —C(O)R^{b};
R³, R⁴, R⁶, and R⁷ are each hydrogen;
R⁸ is, independently, hydrogen or methyl;
R^{a} is, independently, ethyl, isopropyl, or cyclopropyl; and
R^{b} is, independently, 2-methylphenyl, N-methylpyrrol-2-yl, or 3-methoxythien-2-yl.

In some embodiments:
Y is —CR³R⁴—, —NR⁵—, or —O—;
X is —CR⁶R⁷—, —NR⁸—, or —O—;
with the proviso that either Y is —CR³R⁴— or X is —CR⁶R⁷—;
R¹ is hydrogen, C₁₋₆ alkyl, or C₁₋₆ haloalkyl;
R² is —C(O)OR^{a}, —C(O)R^{b}, —C(O)NR^{c}R^{d}, C₃₋₇ cycloalkyl-C₁₋₃alkyl, C₃₋₇ heterocycloalkyl-C₁₋₃ alkyl, C₆₋₁₀ aryl-C₁₋₃alkyl, or C₃₋₉ heteroaryl-C₁₋₃alkyl; wherein the C₆₋₁₀ aryl-C₁₋₃alkyl and C₃₋₉ heteroaryl-C₁₋₃alkyl are each optionally substituted by 1, 2, 3, or 4 independently selected R⁹ groups; and wherein the C₃₋₇ cycloalkyl-C₁₋₃alkyl and C₃₋₇ heterocycloalkyl-C₁₋₃ alkyl are each optionally substituted by 1, 2, 3, or 4 independently selected R¹⁰ groups;
R³, R⁴, R⁶, and R⁷ are each, independently, hydrogen or C₁₋₄alkyl;
R⁵ and R⁸ are each, independently, hydrogen or C₁₋₄ alkyl;
each R⁹ is, independently, halogen, —CN, —NO₂, —OH, C₁₋₆ alkyl, C₁₋₆ haloalkyl, C₁₋₆ alkoxy, C₁₋₆ haloalkoxy. —NR^{e}R^{f}, —(CH₂)ᵣNR^{e}R^{f} or —SO₂R^{e};
each R¹⁰ is, independently, —CN, —NO₂, —OH, C₁₋₆ alkyl, C₁₋₆ haloalkyl, C₁₋₆ alkoxy, C₁₋₆ haloalkoxy, —NR^{e}R^{f}, —(CH₂)ᵣNR^{e}R^{f} or —SO₂R^{e};
R^{a}, R^{b}, R^{c}, and R^{d} are each, independently, hydrogen, C₁₋₇ alkyl, C₂₋₆ alkenyl, C₂₋₆ alkynyl, C₁₋₆ haloalkyl, C₃₋₇ cycloalkyl, C₃₋₇ cycloalkyl-C₁₋₃alkyl, C₃₋₇ heteroalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein said C₆₋₁₀aryl, C₆₋₁₀aryl-C₁₋₃alkyl, C₃₋₉ heteroaryl, and C₃₋₉ heteroaryl-C₁₋₃alkyl are each optionally substituted by 1, 2, 3, or 4 independently selected R¹² groups; wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl, are each optionally substituted by 1, 2, 3, or 4 independently selected R¹³ groups; and wherein the C₁₋₇ alkyl, C₂₋₆ alkenyl, C₂₋₆ alkynyl, C₁₋₆ haloalkyl, C₁₋₇ alkoxy, and C₁₋₆ haloalkoxy are each optionally substituted by 1, 2, or 3 independently selected R¹⁴ groups;
each R¹² is, independently, halogen, —CN, —NO₂, hydroxyl, C₁₋₆ alkyl, C₁₋₆ haloalkyl, C₁₋₆ alkoxy, C₁₋₆ haloalkoxy, —NR (CH₂)NR^{g}R^{h} or —SO₂R^{g};
each R¹³ is, independently, —CN, —NO₂, —OH, C₁₋₆ alkyl, C₁₋₆ haloalkyl, C₁₋₆ alkoxy, C₁₋₆ haloalkoxy, —NR^{g}R^{h}, —(CH₂)NR^{g}R^{h} or —SO₂R^{g};
each R¹⁴ is, independently, —CN, —NO₂, —OH, C₁₋₆ alkoxy, C₁₋₆ haloalkoxy, —NR^{g}R^{h}, —(CH₂)ᵣNR^{g}R^{h} or —SO₂R^{g}; and
each R^{e}, R^{f}, R^{g} and R^{h} is, independently hydrogen or C₁₋₆ alkyl;
   or pharmaceutically acceptable salt thereof.

In some embodiments:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen, C₁₋₆ alkyl, or fluorinated C₁₋₆ haloalkyl;
R² is —C(O)OR^{a}, —C(O)R^{b}, —C(O)NR^{c}R^{d}, C₃₋₇ cycloalkyl-C₁₋₃alkyl, C₃₋₇ heterocycloalkyl-C₁₋₃ alkyl, C₆₋₁₀ aryl-C₁₋₃alkyl, or C₃₋₉ heteroaryl-C₁₋₃alkyl; wherein the C₆₋₁₀ aryl-C₁₋₃alkyl and C₃₋₉ heteroaryl-C₁₋₃alkyl are each optionally substituted by 1, 2, 3, or 4 independently selected R⁹ groups; and wherein the C₃₋₇ cycloalkyl-C₁₋₃alkyl and C₃₋₇ heterocycloalkyl-C₁₋₃ alkyl are each optionally substituted by 1, 2, 3, or 4 independently selected R¹⁰ groups;
R³, R⁴, R⁶, and R⁷ are each hydrogen;
R⁸ is, independently, hydrogen or C₁₋₄ alkyl;
each R⁹ is, independently, halogen, —CN, —NO₂, —OH, C₁₋₄ alkyl, C₁₋₄ haloalkyl, C₁₋₄ alkoxy, or C₁₋₄ haloalkoxy;
each R¹⁰ is, independently, C₁₋₄ alkyl, C₁₋₄ haloalkyl, C₁₋₄ alkoxy, or C₁₋₄ haloalkoxy;
R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀ aryl, C₆₋₁₀ aryl-C₁₋₃ alkyl, C₃₋₉ heteroaryl, or C₃₋₉ heteroaryl-C₁₋₃alkyl; wherein said C₆₋₁₀ aryl, C₆₋₁₀ aryl-C₁₋₃alkyl, C₃₋₉ heteroaryl, and C₃₋₉ heteroaryl-C₁₋₃alkyl are each optionally substituted with 1, 2, or 3 independently selected R¹² groups; and wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl are each optionally substituted with 1, 2, or 3 independently selected R¹³ groups.
each R¹² is, independently, halogen, —CN, —NO₂, —OH, C₁₋₆ alkyl, C₁₋₆ haloalkyl, C₁₋₆ alkoxy, C₁₋₆ haloalkoxy, or —NR^{g}R^{h};
each R¹³ is, independently, C₁₋₆ alkyl, C₁₋₆ haloalkyl, C₁₋₆ alkoxy, or C₁₋₆ haloalkoxy; and
each R^{g} and R^{h} is, independently hydrogen or C₁₋₆ alkyl.

In some embodiments:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen, C₁₋₆ alkyl, or fluorinated C₁₋₆ haloalkyl;
R² is —C(O)OR^{a}, —C(O)R^{b}, —C(O)NR^{c}R^{d}, —CH₂-cycloalkyl, —CH₂—heterocycloalkyl, —CH₂-aryl, or —CH₂-heteroaryl; wherein said —CH₂-aryl and —CH₂-heteroaryl are each optionally substituted by 1, 2, 3, or 4 independently selected R⁹ groups;
R³, R⁴, R⁶, and R⁷ are each hydrogen;
R⁸ is, independently, hydrogen or C₁₋₄ alkyl;
each R⁹ is, independently, halogen, —CN, —NO₂, —OH, C₁₋₄ alkyl, C₁₋₄ haloalkyl, C₁₋₄ alkoxy, or C₁₋₄ haloalkoxy;
each R¹⁰ is, independently, C₁₋₄ alkyl, C₁₋₄ haloalkyl, C₁₋₄ alkoxy, or C₁₋₄ haloalkoxy;
R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀ aryl, C₆₋₁₀ aryl-C₁₋₃ alkyl, C₃₋₉ heteroaryl, or C₃₋₉ heteroaryl-C₁₋₃alkyl; wherein said C₆₋₁₀ aryl, C₆₋₁₀ aryl-C₁₋₃alkyl, C₃₋₉ heteroaryl, and C₃₋₉ heteroaryl-C₁₋₃alkyl are each optionally substituted with 1, 2, or 3 independently selected R¹² groups; and wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl are each optionally substituted with 1, 2, or 3 independently selected R¹³ groups;
each R¹² is, independently, halogen, CN, —NO₂, —OH, C₁₋₆ alkyl, C₁₋₆ haloalkyl, C₁₋₆ alkoxy, C₁₋₆ haloalkoxy, or —NR^{g}R^{h}; and
each R¹³ is, independently, C₁₋₆ alkyl, C₁₋₆ haloalkyl, C₁₋₆ alkoxy, or C₁₋₆ haloalkoxy; and
each R^{g} and R^{h} is, independently hydrogen or C₁₋₆ alkyl.

In some embodiments:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen, C₁₋₃ alkyl, or fluorinated C₁₋₃ haloalkyl;
R² is —C(O)OR^{a}, —C(O)R^{b}, —C(O)NR^{c}R^{d}, C₆₋₁₀aryl-C₁₋₃alkyl or C₃₋₉heteroaryl-C₁₋₃alkyl; wherein said C₆₋₁₀aryl-C₁₋₃alkyl and C₃₋₉heteroaryl-C₁₋₃alkyl are each optionally substituted by 1, 2, or 3 independently selected R⁹ groups;
R³, R⁴, R⁶, and R⁷ are each hydrogen;
R⁸ is, independently, hydrogen or C₁₋₃ alkyl;
each R⁹ is, independently, C₁₋₄ alkyl, C₁₋₄ haloalkyl, C₁₋₄ alkoxy, and C₁₋₄ haloalkyl;
R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₆₋₁₀ aryl, or C₃₋₉ heteroaryl; wherein said C₆₋₁₀ aryl and C₃₋₉ heteroaryl are each optionally substituted with 1, 2, or 3 independently selected R¹² groups; and
each R¹² is, independently, C₁₋₆ alkyl, C₁₋₆ haloalkyl, C₁₋₆ alkoxy, or C₁₋₆ haloalkoxy.

In some embodiments:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen, C₁₋₃ alkyl, or fluorinated C₁₋₃ haloalkyl;
R² is —C(O)OR^{a}, —C(O)R^{b}, —C(O)NR^{c}R^{d}, —CH₂—C₆₋₁₀aryl, or —CH₂—C₆₋₉heteroaryl; wherein said —CH₂—C₆₋₁₀aryl and —CH₂—C₆₋₉heteroaryl are each optionally substituted by 1, 2, 3, or 4 independently selected R⁹ groups;
R³, R⁴, R⁶, and R⁷ are each hydrogen;
R⁸ is, independently, hydrogen or C₁₋₃ alkyl;
each R⁹ is, independently, C₁₋₄ alkyl, C₁₋₄ haloalkyl, C₁₋₄ alkoxy, and C₁₋₄ haloalkyl;
R^{a}, R^{b}, R^{c}, and R^{d} are each, independently, C₁₋₇ alkyl, —CH₂—(C₂₋₅ alkynyl), C₁₋₆ haloalkyl, C₃₋₇ cycloalkyl, C₆₋₁₀ aryl, or C₃₋₉ heteroaryl; wherein said C₆₋₁₀ aryl and C₃₋₉ heteroaryl are each optionally substituted with 1, 2, or 3 independently selected R¹² groups; and
each R¹² is, independently, C₁₋₆ alkyl, C₁₋₆ haloalkyl, C₁₋₆ alkoxy, or C₁₋₆ haloalkoxy.

In some embodiments, the compound is a compound of Formula II or III, or pharmaceutically acceptable salt thereof:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen, C₁₋₃ alkyl, fluoromethyl, difluoromethyl, or trifluoromethyl;
R² is —C(O)OR^{a}, and —C(O)R^{b};
R³, R⁴, R⁶, and R⁷ are each hydrogen;
R⁸ is, independently, hydrogen or C₁₋₂ alkyl;
R^{a}, R^{b}, R^{c}, and R^{d} are each, independently, C₁₋₇ alkyl, C₁₋₆ haloalkyl, C₃₋₇ cycloalkyl, phenyl, or C₃₋₇ heteroaryl; wherein said phenyl or C₃₋₉ heteroaryl is each optionally substituted with 1 or 2 independently selected R¹² groups; and

each R¹² is, independently, C₁₋₆ alkyl, C₁₋₆ haloalkyl, C₁₋₆ alkoxy, or C₁₋₆ haloalkoxy.

In some embodiments, the compound is a compound of Formula II or III:

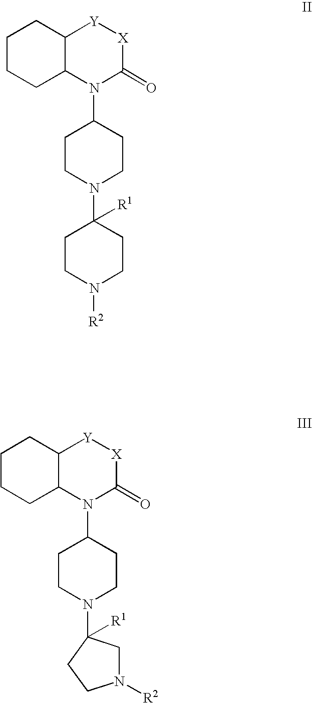

or pharmaceutically acceptable salt thereof.

In some embodiments, the compound is a compound of Formula IV, V, VI, VII, or VIII:

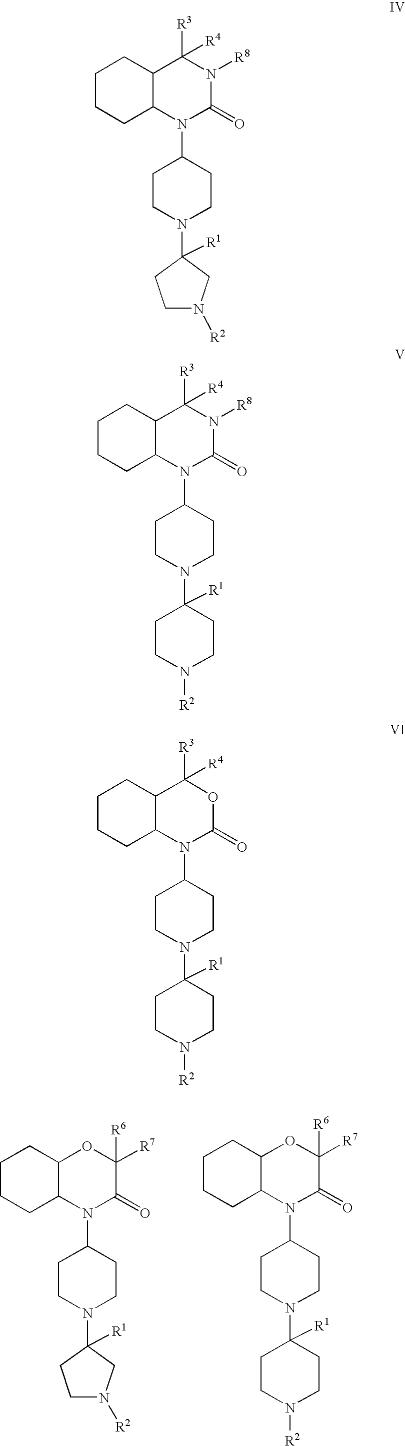

### VII VIII

or pharmaceutically acceptable salt thereof.

In some embodiments, the compound is a compound of Formula X, XI, XII, XIII, XIV, or XV:

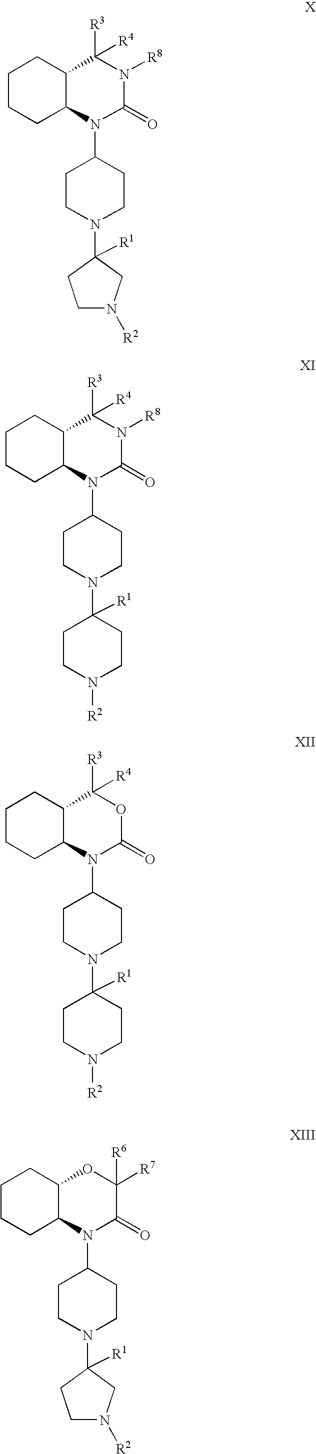

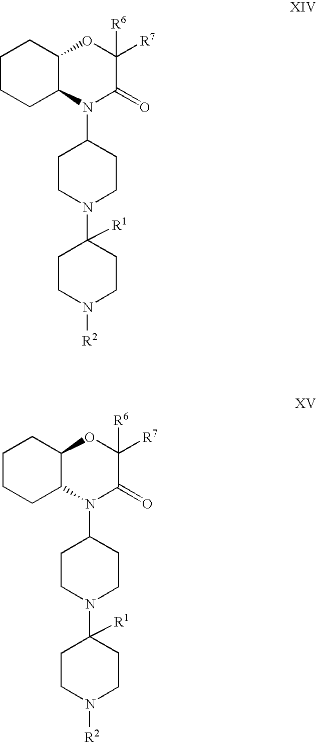

or pharmaceutically acceptable salt thereof.

In some embodiments, the compound is a compound of Formula II or III:

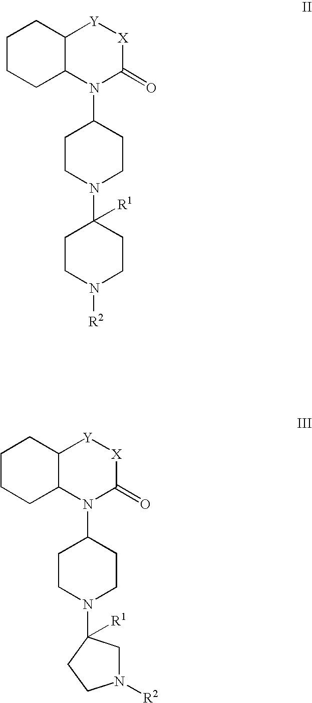

wherein:
Y is —CR³R⁴—, —NR⁵—, or —O—;
X is —CR⁶R⁷—, —NR⁸—, or —O—;
with the proviso that either Y is —CR³R⁴— or X is —CR⁶R⁷—;
R¹ is hydrogen or C₁₋₆ alkyl;
R² is —C(O)OR^{a}, —C(O)R^{b}, —C(O)NR^{c}R^{d}, C₃₋₇ cycloalkyl-C₁₋₃alkyl, C₃₋₇ heterocycloalkyl-C₁₋₃ alkyl, C₆₋₁₀ aryl-C₁₋₃alkyl, or C₃₋₉ heteroaryl-C₁₋₃alkyl; wherein the C₆₋₁₀ aryl-C₁₋₃alkyl and C₃₋₉ heteroaryl-C₁₋₃alkyl are each optionally substituted by 1, 2, 3, or 4 independently selected R⁹ groups; and wherein the C₃₋₇ cycloalkyl-C₁₋₃alkyl and C₃₋₇ heterocycloalkyl-C₁₋₃ alkyl are each optionally substituted by 1, 2, 3, or 4 independently selected R¹⁰ groups;
R³, R⁴, R⁶, and R⁷ are each, independently, hydrogen, fluoro, C₁₋₄ alkyl, C₁₋₄ alkoxymethyl, cyanoC₁₋₄ alkyl or C₁₋₄ haloalkyl; R⁵ and R⁸ are each, independently, hydrogen or C₁₋₄ alkyl;
each R⁹ is, independently, halogen, —CN, —NO₂, —OH, C₁₋₆ alkyl, C₁₋₆ haloalkyl, C₁₋₆ alkoxy, C₁₋₆ haloalkoxy. —NR^{e}R^{f}, —(CH₂)ᵣNR^{e}R^{f} or —SO₂R^{e};
each R¹⁰ is, independently, —CN, —NO₂, —OH, C₁₋₆ alkyl, C₁₋₆ haloalkyl, C₁₋₆ alkoxy, C₁₋₆ haloalkoxy, —NR^{e}R^{f}, —(CH₂)ᵣNR^{e}R^{f} or —SO₂R^{e};
R^{a}, R^{b}, R^{c}, and R^{d} are each, independently, hydrogen, C₁₋₇ alkyl, C₂₋₆ alkenyl, C₂₋₆ alkynyl, C₁₋₆ haloalkyl, C₃₋₇ cycloalkyl, C₃₋₇ cycloalkyl-C₁₋₃alkyl, C₃₋₇ heteroalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein said C₆₋₁₀aryl, C₆₋₁₀aryl-C₁₋₃alkyl, C₃₋₉ heteroaryl, and C₃₋₉ heteroaryl-C₁₋₃alkyl are each optionally substituted by 1, 2, 3, or 4 independently selected R¹² groups; wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl, are each optionally substituted by 1, 2, 3, or 4 independently selected R¹³ groups; and wherein the C₁₋₇ alkyl, C₂₋₆ alkenyl, C₂₋₆ alkynyl, C₁₋₆ haloalkyl, C₁₋₇ alkoxy, and C₁₋₆ haloalkoxy are each optionally substituted by 1, 2, or 3 independently selected R¹⁴ groups;
each R¹² is, independently, halogen, —CN, —NO₂, hydroxyl, C₁₋₆ alkyl, C₁₋₆ haloalkyl, C₁₋₆ alkoxy, C₁₋₆ haloalkoxy, —NR^{g}R^{h}, —(CH₂)ᵣNR^{g}R^{h} or —SO₂R^{g};
each R¹³ is, independently, —CN, —NO₂, —OH, C₁₋₆ alkyl, C₁₋₆ haloalkyl, C₁₋₆ alkoxy, C₁₋₆ haloalkoxy, —NR^{g}R^{h}, —(CH₂)ᵣNR⁹R^{h} or —SO₂R^{g}.
each R¹⁴ is, independently, —CN, —NO₂, —OH, C₁₋₆ alkoxy, C₁₋₆ haloalkoxy, —NR^{g}R^{h}, —(CH₂)ᵣNR^{g}R^{h} or —SO₂R^{g}; and
each R^{e}, R^{f}, R^{g} and R^{h} is, independently hydrogen or C₁₋₆ alkyl;
   or pharmaceutically acceptable salt thereof.

In some embodiments, the compound is a compound of Formula II or III, or pharmaceutically acceptable salt thereof:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or C₁₋₆ alkyl;
R² is —C(O)OR^{a}, —C(O)R^{b}, —C(O)NR^{c}R^{d}, C₃₋₇ cycloalkyl-C₁₋₃alkyl, C₃₋₇ heterocycloalkyl-C₁₋₃ alkyl, C₆₋₁₀ aryl-C₁₋₃alkyl, or C₃₋₉ heteroaryl-C₁₋₃alkyl; wherein the C₆₋₁₀ aryl-C₁₋₃alkyl and C₃₋₉ heteroaryl-C₁₋₃alkyl are each optionally substituted by 1, 2, 3, or 4 independently selected R⁹ groups; and wherein the C₃₋₇ cycloalkyl-C₁₋₃alkyl and C₃₋₇ heterocycloalkyl-C₁₋₃ alkyl are each optionally substituted by 1, 2, 3, or 4 independently selected R¹⁰ groups;
R³, R⁴, R⁶, and R⁷ are each hydrogen;
R⁸ is, independently, hydrogen or C₁₋₄ alkyl;
each R⁹ is, independently, halogen, —CN, —NO₂, —OH, C₁₋₄ alkyl, C₁₋₄ haloalkyl, C₁₋₄ alkoxy, or C₁₋₄ haloalkoxy;
each R¹⁰ is, independently, C₁₋₄ alkyl, C₁₋₄ haloalkyl, C₁₋₄ alkoxy, or C₁₋₄ haloalkoxy;
R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀ aryl, C₆₋₁₀ aryl-C₁₋₃ alkyl, C₃₋₉ heteroaryl, or C₃₋₉ heteroaryl-C₁₋₃alkyl; wherein said C₆₋₁₀ aryl, C₆₋₁₀ aryl-C₁₋₃alkyl, C₃₋₉ heteroaryl, and C₃₋₉ heteroaryl-C₁₋₃alkyl are each optionally substituted with 1, 2, or 3 independently selected R¹² groups; and wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl are each optionally substituted with 1, 2, or 3 independently selected R¹³ groups.
each R¹² is, independently, halogen, —CN, —NO₂, —OH, C₁₋₆ alkyl, C₁₋₆ haloalkyl, C₁₋₆ alkoxy, C₁₋₆ haloalkoxy, or —NR^{g}R^{h};
each R¹³ is, independently, C₁₋₆ alkyl, C₁₋₆ haloalkyl, C₁₋₆ alkoxy, or C₁₋₆ haloalkoxy; and
each R^{g} and R^{h} is, independently hydrogen or C₁₋₆ alkyl.

In some embodiments, the compound is a compound of Formula II or III, or pharmaceutically acceptable salt thereof:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or C₁₋₆ alkyl;
R² is —C(O)OR^{a}, —C(O)R^{b}, —C(O)NR^{c}R^{d}, —CH₂-cycloalkyl, —CH₂—heterocycloalkyl, —CH₂-aryl, or —CH₂-heteroaryl; wherein said —CH₂-aryl and —CH₂-heteroaryl are each optionally substituted by 1, 2, 3, or 4 independently selected R⁹ groups;
R³, R⁴, R⁶, and R⁷ are each hydrogen;
R⁸ is, independently, hydrogen or C₁₋₄ alkyl;
each R⁹ is, independently, halogen, —CN, —NO₂, —OH, C₁₋₄ alkyl, C₁₋₄ haloalkyl, C₁₋₄ alkoxy, or C₁₋₄ haloalkoxy;
each R¹⁰ is, independently, C₁₋₄ alkyl, C₁₋₄ haloalkyl, C₁₋₄ alkoxy, or C₁₋₄ haloalkoxy;
R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀ aryl, C₆₋₁₀ aryl-C₁₋₃ alkyl, C₃₋₉ heteroaryl, or C₃₋₉ heteroaryl-C₁₋₃alkyl; wherein said C₆₋₁₀ aryl, C₆₋₁₀ aryl-C₁₋₃alkyl, C₃₋₉ heteroaryl, and C₃₋₉ heteroaryl-C₁₋₃alkyl are each optionally substituted with 1, 2, or 3 independently selected R¹² groups; and wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl are each optionally substituted with 1, 2, or 3 independently selected R¹³ groups;
each R¹² is, independently, halogen, CN, —NO₂, —OH, C₁₋₆ alkyl, C₁₋₆ haloalkyl, C₁₋₆ alkoxy, C₁₋₆ haloalkoxy, or —NR^{g}R^{h}; and
each R¹³ is, independently, C₁₋₆ alkyl, C₁₋₆ haloalkyl, C₁₋₆ alkoxy, or C₁₋₆ haloalkoxy; and
each R^{g} and R^{h} is, independently hydrogen or C₁₋₆ alkyl.

In some embodiments, the compound is a compound of Formula II or III, or pharmaceutically acceptable salt thereof:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or C₁₋₃ alkyl;
R² is —C(O)OR^{a}, —C(O)R^{b}, —C(O)NR^{c}R^{d}, C₆₋₁₀aryl-C₁₋₃alkyl or C₃₋₉heteroaryl-C₁₋₃alkyl; wherein said C₆₋₁₀aryl-C₁₋₃alkyl and C₃₋₉heteroaryl-C₁₋₃alkyl are each optionally substituted by 1, 2, or 3 independently selected R⁹ groups;
R³, R⁴, R⁶, and R⁷ are each hydrogen;
R⁸ is, independently, hydrogen or C₁₋₃ alkyl;
each R⁹ is, independently, C₁₋₄ alkyl, C₁₋₄ haloalkyl, C₁₋₄ alkoxy, and C₁₋₄ haloalkyl;
R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₆₋₁₀ aryl, or C₃₋₉ heteroaryl; wherein said C₆₋₁₀ aryl and C₃₋₉ heteroaryl are each optionally substituted with 1, 2, or 3 independently selected R¹² groups; and
each R¹² is, independently, C₁₋₆ alkyl, C₁₋₆ haloalkyl, C₁₋₆ alkoxy, or C₁₋₆ haloalkoxy.

In some embodiments, the compound is a compound of Formula II or III, or pharmaceutically acceptable salt thereof:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or C₁₋₃ alkyl;
R² is —C(O)OR^{a}, —C(O)R^{b}, —C(O)NR^{c}R^{d}, —CH₂—C₆₋₁₀aryl, or —CH₂—C₆₋₉heteroaryl; wherein said —CH₂—C₆₋₁₀aryl and —CH₂—C₆₋₉heteroaryl are each optionally substituted by 1, 2, 3, or 4 independently selected R⁹ groups;
R³, R⁴, R⁶, and R⁷ are each hydrogen;
R⁸ is, independently, hydrogen or C₁₋₃ alkyl;
each R⁹ is, independently, C₁₋₄ alkyl, C₁₋₄ haloalkyl, C₁₋₄ alkoxy, and C₁₋₄ haloalkyl;
R^{a}, R^{b}, R^{c}, and R^{d} are each, independently, C₁₋₇ alkyl, —CH₂—(C₂₋₅ alkynyl), C₁₋₆ haloalkyl, C₃₋₇ cycloalkyl, C₆₋₁₀ aryl, or C₃₋₉ heteroaryl; wherein said C₆₋₁₀ aryl and C₃₋₉ heteroaryl are each optionally substituted with 1, 2, or 3 independently selected R¹² groups; and
each R¹² is, independently, C₁₋₆ alkyl, C₁₋₆ haloalkyl, C₁₋₆ alkoxy, or C₁₋₆ haloalkoxy.

In some embodiments, the compound is a compound of Formula II or III, or pharmaceutically acceptable salt thereof:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or C₁₋₃ alkyl;
R² is —C(O)OR^{a}, and —C(O)R^{b};
R³, R⁴, R⁶, and R⁷ are each hydrogen;
R⁸ is, independently, hydrogen or C₁₋₂ alkyl;
R^{a}, R^{b}, R^{c}, and R^{d} are each, independently, C₁₋₇ alkyl, C₁₋₆ haloalkyl, C₃₋₇ cycloalkyl, phenyl, or C₃₋₇ heteroaryl; wherein said phenyl or C₃₋₉ heteroaryl is each optionally substituted with 1 or 2 independently selected R¹² groups; and
each R¹² is, independently, C₁₋₆ alkyl, C₁₋₆ haloalkyl, C₁₋₆ alkoxy, or C₁₋₆ haloalkoxy.

In some embodiments, the compound is a compound of Formula II or III, or pharmaceutically acceptable salt thereof:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or C₁₋₃ alkyl;
R² is —C(O)OR^{a}, and —C(O)R^{b};
R³, R⁴, R⁶, and R⁷ are each hydrogen;
R⁸ is, independently, hydrogen or C₁₋₃ alkyl;
R^{a} and R^{b} are each, independently, C₁₋₄ alkyl, C₁₋₄ haloalkyl, C₃₋₇ cycloalkyl, phenyl, or C₃₋₉ heteroaryl; wherein said phenyl or C₃₋₉ heteroaryl is each optionally substituted with 1 or 2 independently selected R¹² groups; and
each R¹² is, independently, C₁₋₆ alkyl or C₁₋₆ alkoxy.

In some embodiments, the compound is a compound of Formula II or III, or pharmaceutically acceptable salt thereof:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or methyl; and
R² is —C(O)OR^{a}, and —C(O)R^{b};
R³, R⁴, R⁶, and R⁷ are each hydrogen;
R⁸ is, independently, hydrogen or methyl;
R^{a} is, independently, ethyl, isopropyl, or cyclopropyl;
R^{b} is, independently, phenyl, pyrrolyl, or thienyl, wherein the phenyl, pyrrolyl or thienyl is optionally substituted with 1 R¹² group; and
each R¹² is, independently, methoxy or methyl.

In some embodiments, the compound is a compound of Formula II or III, or pharmaceutically acceptable salt thereof, wherein:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen or methyl; and
R² is —C(O)OR^{a}, and —C(O)R^{b};
R³, R⁴, R⁶, and R⁷ are each hydrogen;
R⁸ is, independently, hydrogen or methyl;
R^{a} is, independently, ethyl, isopropyl, or cyclopropyl; and
R^{b} is, independently, 2-methylphenyl, N-methylpyrrol-2-yl, or 3-methoxythien-2-yl.

In some embodiments, the compound is a compound of Formula II or III, or pharmaceutically acceptable salt thereof, wherein:
Y is —CR³R⁴—, —NR⁵—, or —O—;
X is —CR⁶R⁷—, —NR⁸—, or —O—;
with the proviso that either Y is —CR³R⁴— or X is —CR⁶R⁷—;
R¹ is hydrogen, C₁₋₆ alkyl, or C₁₋₆ haloalkyl;
R² is —C(O)OR^{a}, —C(O)R^{b}, —C(O)NR^{c}R^{d}, C₃₋₇ cycloalkyl-C₁₋₃alkyl, C₃₋₇ heterocycloalkyl-C₁₋₃ alkyl, C₆₋₁₀ aryl-C₁₋₃alkyl, or C₃₋₉ heteroaryl-C₁₋₃alkyl; wherein the C₆₋₁₀ aryl-C₁₋₃alkyl and C₃₋₉ heteroaryl-C₁₋₃alkyl are each optionally substituted by 1, 2, 3, or 4 independently selected R⁹ groups; and wherein the C₃₋₇ cycloalkyl-C₁₋₃alkyl and C₃₋₇ heterocycloalkyl-C₁₋₃ alkyl are each optionally substituted by 1, 2, 3, or 4 independently selected R¹⁰ groups;
R³, R⁴, R⁶, and R⁷ are each, independently, hydrogen or C₁₋₄ alkyl;
R⁵ and R⁸ are each, independently, hydrogen or C₁₋₄ alkyl;
each R⁹ is, independently, halogen, —CN, —NO₂, —OH, C₁₋₆ alkyl, C₁₋₆ haloalkyl, C₁₋₆ alkoxy, C₁₋₆ haloalkoxy. —NR^{e}R^{f}, —(CH₂)ᵣNR^{e}R^{f} or —SO₂R^{e};
each R¹⁰ is, independently, —CN, —NO₂, —OH, C₁₋₆ alkyl, C₁₋₆ haloalkyl, C₁₋₆ alkoxy, C₁₋₆ haloalkoxy, —NR^{e}R^{f}, —(CH₂)ᵣNR^{e}R^{f} or —SO₂R^{e};
R^{a}, R^{b}, R^{c}, and R^{d} are each, independently, hydrogen, C₁₋₇ alkyl, C₂₋₆ alkenyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein said C₆₋₁₀aryl, C₆₋₁₀aryl-C₁₋₃alkyl, C₃₋₉ heteroaryl, and C₃₋₉ heteroaryl-C₁₋₃alkyl are each optionally substituted by 1, 2, 3, or 4 independently selected R¹² groups; wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl, are each optionally substituted by 1, 2, 3, or 4 independently selected R¹³ groups; and wherein the C₁₋₇ alkyl, C₂₋₆ alkenyl, C₂₋₆ alkynyl, C₁₋₆ haloalkyl, C₁₋₇ alkoxy, and C₁₋₆ haloalkoxy are each optionally substituted by 1, 2, or 3 independently selected R¹⁴ groups;
each R¹² is, independently, halogen, —CN, —NO₂, hydroxyl, C₁₋₆ alkyl, C₁₋₆ haloalkyl, C₁₋₆ alkoxy, C₁₋₆ haloalkoxy, —NR^{g}R^{h}, —(CH₂)ᵣNR^{g}R^{h} or —SO₂R^{g};
each R¹³ is, independently, —CN, —NO₂, —OH, C₁₋₆ alkyl, C₁₋₆ haloalkyl, C₁₋₆ alkoxy, C₁₋₆ haloalkoxy, —NR^{g}R^{h}, —(CH₂)ᵣNR^{g}R^{h} or —SO₂R^{g}.
each R¹⁴ is, independently, —CN, —NO₂, —OH, C₁₋₆ alkoxy, C₁₋₆ haloalkoxy, —NR^{g}R^{h}, —(CH₂)ᵣNR^{g}R^{h} or —SO₂R^{g}; and
each R^{e}, R^{f}, R^{g} and R^{h} is, independently hydrogen or C₁₋₆ alkyl;
   or pharmaceutically acceptable salt thereof.

In some embodiments, the compound is a compound of Formula II or III, or pharmaceutically acceptable salt thereof:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen, C₁₋₆ alkyl, or fluorinated C₁₋₆ haloalkyl;
R² is —C(O)OR^{a}, —C(O)R^{b}, —C(O)NR^{c}R^{d}, C₃₋₇ cycloalkyl-C₁₋₃alkyl, C₃₋₇ heterocycloalkyl-C₁₋₃ alkyl, C₆₋₁₀ aryl-C₁₋₃alkyl, or C₃₋₉ heteroaryl-C₁₋₃alkyl; wherein the C₆₋₁₀ aryl-C₁₋₃alkyl and C₃₋₉ heteroaryl-C₁₋₃alkyl are each optionally substituted by 1, 2, 3, or 4 independently selected R⁹ groups; and wherein the C₃₋₇ cycloalkyl-C₁₋₃alkyl and C₃₋₇ heterocycloalkyl-C₁₋₃ alkyl are each optionally substituted by 1, 2, 3, or 4 independently selected R¹⁰ groups;
R³, R⁴, R⁶, and R⁷ are each hydrogen;
R⁸ is, independently, hydrogen or C₁₋₄ alkyl;
each R⁹ is, independently, halogen, —CN, —NO₂, —OH, C₁₋₄ alkyl, C₁₋₄ haloalkyl, C₁₋₄ alkoxy, or C₁₋₄ haloalkoxy;
each R¹⁰ is, independently, C₁₋₄ alkyl, C₁₋₄ haloalkyl, C₁₋₄ alkoxy, or C₁₋₄ haloalkoxy;
R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀ aryl, C₆₋₁₀ aryl-C₁₋₃ alkyl, C₃₋₉ heteroaryl, or C₃₋₉ heteroaryl-C₁₋₃alkyl; wherein said C₆₋₁₀ aryl, C₆₋₁₀ aryl-C₁₋₃alkyl, C₃₋₉ heteroaryl, and C₃₋₉ heteroaryl-C₁₋₃alkyl are each optionally substituted with 1, 2, or 3 independently selected R¹² groups; and wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl are each optionally substituted with 1, 2, or 3 independently selected R¹³ groups.
each R¹² is, independently, halogen, —CN, —NO₂, —OH, C₁₋₆ alkyl, C₁₋₆ haloalkyl, C₁₋₆ alkoxy, C₁₋₆ haloalkoxy, or —NR^{g}R^{h};
each R¹³ is, independently, C₁₋₆ alkyl, C₁₋₆ haloalkyl, C₁₋₆ alkoxy, or C₁₋₆ haloalkoxy; and
each R^{g} and R^{h} is, independently hydrogen or C₁₋₆ alkyl.

In some embodiments, the compound is a compound of Formula II or III, or pharmaceutically acceptable salt thereof:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen, C₁₋₆ alkyl, or fluorinated C₁₋₆ haloalkyl;
R² is —C(O)OR^{a}, —C(O)R^{b}, —C(O)NR^{c}R^{d}, —CH₂-cycloalkyl, —CH₂-heterocycloalkyl, —CH₂-aryl, or —CH₂-heteroaryl; wherein said —CH₂-aryl and —CH₂-heteroaryl are each optionally substituted by 1, 2, 3, or 4 independently selected R⁹ groups;
R³, R⁴, R⁶, and R⁷ are each hydrogen;
R⁸ is, independently, hydrogen or C₁₋₄ alkyl;
each R⁹ is, independently, halogen, —CN, —NO₂, —OH, C₁₋₄ alkyl, C₁₋₄ haloalkyl, C₁₋₄ alkoxy, or C₁₋₄ haloalkoxy;
each R¹⁰ is, independently, C₁₋₄ alkyl, C₁₋₄ haloalkyl, C₁₋₄ alkoxy, or C₁₋₄ haloalkoxy;
R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀ aryl, C₆₋₁₀ aryl-C₁₋₃ alkyl, C₃₋₉ heteroaryl, or C₃₋₉ heteroaryl-C₁₋₃alkyl; wherein said C₆₋₁₀ aryl, C₆₋₁₀ aryl-C₁₋₃alkyl, C₃₋₉ heteroaryl, and C₃₋₉ heteroaryl-C₁₋₃alkyl are each optionally substituted with 1, 2, or 3 independently selected R¹² groups; and wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl are each optionally substituted with 1, 2, or 3 independently selected R¹³ groups;
each R¹² is, independently, halogen, CN, —NO₂, —OH, C₁₋₆ alkyl, C₁₋₆ haloalkyl, C₁₋₆ alkoxy, C₁₋₆ haloalkoxy, or —NR^{g}R^{h}; and
each R¹³ is, independently, C₁₋₆ alkyl, C₁₋₆ haloalkyl, C₁₋₆ alkoxy, or C₁₋₆ haloalkoxy; and
each R^{g} and R^{h} is, independently hydrogen or C₁₋₆ alkyl.

In some embodiments, the compound is a compound of Formula II or III, or pharmaceutically acceptable salt thereof:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen, C₁₋₃ alkyl, or fluorinated C₁₋₃ haloalkyl;
R² is —C(O)OR^{a}, —C(O)R^{b}, —C(O)NR^{c}R^{d}, C₆₋₁₀aryl-C₁₋₃alkyl or C₃₋₉heteroaryl-C₁₋₃alkyl; wherein said C₆₋₁₀aryl-C₁₋₃alkyl and C₃₋₉heteroaryl-C₁₋₃alkyl are each optionally substituted by 1, 2, or 3 independently selected R⁹ groups;
R³, R⁴, R⁶, and R⁷ are each hydrogen;
R⁸ is, independently, hydrogen or C₁₋₃ alkyl;
each R⁹ is, independently, C₁₋₄ alkyl, C₁₋₄ haloalkyl, C₁₋₄ alkoxy, and C₁₋₄ haloalkyl;
R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₆₋₁₀ aryl, or C₃₋₉ heteroaryl; wherein said C₆₋₁₀ aryl and C₃₋₉ heteroaryl are each optionally substituted with 1, 2, or 3 independently selected R¹² groups; and
each R¹² is, independently, C₁₋₆ alkyl, C₁₋₆ haloalkyl, C₁₋₆ alkoxy, or C₁₋₆ haloalkoxy.

In some embodiments, the compound is a compound of Formula II or III, or pharmaceutically acceptable salt thereof:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen, C₁₋₃ alkyl, or fluorinated C₁₋₃ haloalkyl;
R² is —C(O)OR^{a}, —C(O)R^{b}, —C(O)NR^{c}R^{d}, —CH₂—C₆₋₁₀aryl, or —CH₂—C₆₋₉heteroaryl; wherein said —CH₂—C₆₋₁₀aryl and —CH₂—C₆₋₉heteroaryl are each optionally substituted by 1, 2, 3, or 4 independently selected R⁹ groups;
R³, R⁴, R⁶, and R⁷ are each hydrogen;
R⁸ is, independently, hydrogen or C₁₋₃ alkyl;
each R⁹ is, independently, C₁₋₄ alkyl, C₁₋₄ haloalkyl, C₁₋₄ alkoxy, and C₁₋₄ haloalkyl;
R^{a}, R^{b}, R^{c}, and R^{d} are each, independently, C₁₋₇ alkyl, —CH₂—(C₂₋₅ alkynyl), C₁₋₆ haloalkyl, C₃₋₇ cycloalkyl, C₆₋₁₀ aryl, or C₃₋₉ heteroaryl; wherein said C₆₋₁₀ aryl and C₃₋₉ heteroaryl are each optionally substituted with 1, 2, or 3 independently selected R¹² groups; and
each R¹² is, independently, C₁₋₆ alkyl, C₁₋₆ haloalkyl, C₁₋₆ alkoxy, or C₁₋₆ haloalkoxy.

In some embodiments, the compound is a compound of Formula II or III, or pharmaceutically acceptable salt thereof:
Y is —CR³R⁴— or —O—;
X is —CR⁶R⁷—, —NR⁸—, or —O—;
with the proviso that either Y is —CR³R⁴— or X is —CR⁶R⁷—;
R¹ is hydrogen, C₁₋₃ alkyl, fluoromethyl, difluoromethyl or trifluoromethyl;
R² is —C(O)OR^{a}, and —C(O)R^{b};
R³, R⁴, R⁶, and R⁷ are each hydrogen;
R⁸ is, independently, hydrogen or C₁₋₂ alkyl;
R^{a}, R^{b}, R^{c}, and R^{d} are each, independently, C₁₋₇ alkyl, C₁₋₆ haloalkyl, C₃₋₇ cycloalkyl, phenyl, or C₃₋₇ heteroaryl; wherein said phenyl or C₃₋₉ heteroaryl is each optionally substituted with 1 or 2 independently selected R¹² groups; and
each R¹² is, independently, C₁₋₆ alkyl, C₁₋₆ haloalkyl, C₁₋₆ alkoxy, or C₁₋₆ haloalkoxy.

In some embodiments of each of the previous embodiments, each of the R⁹ groups optionally substitute the rings of the C₆₋₁₀ aryl-C₁₋₃alkyl and C₃₋₉ heteroaryl-C₁₋₃alkyl groups; each of the R¹⁰ groups optionally substitute the C₃₋₇ cycloalkyl-C₁₋₃alkyl and C₃₋₇ heterocycloalkyl-C₁₋₃ alkyl groups; each of the R¹² groups optionally substitute the rings of the C₆₋₁₀ aryl-C₁₋₃alkyl and C₃₋₉ heteroaryl-C₁₋₃alkyl groups; and each of the R¹³ groups optionally substitute the C₃₋₇ cycloalkyl-C₁₋₃alkyl and C₃₋₇ heterocycloalkyl-C₁₋₃ alkyl groups.

In some embodiments, the compound is selected from:
Ethyl 4-[4-[(4aR,8aS)-2-oxo-3,4,4a,5,6,7,8,8a-octahydroquinazolin-1-yl]-1-piperidyl]piperidine-1-carboxylate;
Propan-2-yl 4-[4-[(4aR,8aS)-2-oxo-3,4,4a,5,6,7,8,8a-octahydroquinazolin-1-yl]-1-piperidyl]piperidine-1-carboxylate;
(4aR, 8aS)-1-[1-[1-(Cyclopropanecarbonyl)-4-piperidyl]-4-piperidyl]-3,4,4a,5,6,7,8,8a-octahydroquinazolin-2-one;
(4aR,8aS)-1-[1-[1-(2-Methylbenzoyl)-4-piperidyl]-4-piperidyl]-3,4,4a,5,6,7,8,8a-octahydroquinazolin-2-one;
Ethyl 3-[4-[(4aR,8aS)-2-oxo-3,4,4a,5,6,7,8,8a-octahydroquinazolin-1-yl]-1-piperidyl]pyrrolidine-1-carboxylate;
Propan-2-yl 4-[4-[(4aR,8aS)-3-methyl-2-oxo-4a,5,6,7,8,8a-hexahydro-4H-quinazolin-1-yl]-1-piperidyl]piperidine-1-carboxylate;
Ethyl 4-[4-[(4aR,8aS)-2-oxo-3,4,4a,5,6,7,8,8a-octahydroquinazolin-1-yl]-1-piperidyl]-4-methyl-piperidine-1-carboxylate;
Propan-2-yl 4-[4-[(4aR,8aS)-2-oxo-3,4,4a,5,6,7,8,8a-octahydroquinazolin-1-yl]-1-piperidyl]-4-methyl-piperidine-1-carboxylate;
Ethyl 4-[4-[(1S,6S)-9-oxo-7-oxa-10-azabicyclo[4.4.0]dec-10-yl]-1-piperidyl]piperidine-1-carboxylate;
Propan-2-yl 4-[4-[(1S,6S)-9-oxo-7-oxa-10-azabicyclo[4.4.0]dec-10-yl]-1-piperidyl]piperidine-1-carboxylate;
(1S,6S)-10-[1-[1-(2-Methylbenzoyl)-4-piperidyl]-4-piperidyl]-7-oxa-10-azabicyclo[4.4.0]decan-9-one;
(1S,6S)-10-[1-[1-(1-Methylpyrrole-2-carbonyl)-4-piperidyl]-4-piperidyl]-7-oxa-10-azabicyclo[4.4.0]decan-9-one;
Ethyl (3S)-3-[4-[(1S,6S)-9-oxo-7-oxa-10-azabicyclo[4.4.0]dec-10-yl]-1-piperidyl]pyrrolidine-1-carboxylate;
Propan-2-yl 4-[4-[(1R,6R)-9-oxo-7-oxa-10-azabicyclo[4.4.0]dec-10-yl]-1-piperidyl]piperidine-1-carboxylate;
Ethyl 4-[4-[(1S,6S)-9-oxo-8-oxa-10-azabicyclo[4.4.0]dec-10-yl]-1-piperidyl]piperidine-1-carboxylate;
Propan-2-yl 4-[4-[(1S,6S)-9-oxo-8-oxa-10-azabicyclo[4.4.0]dec-10-yl]-1-piperidyl]piperidine-1-carboxylate;
(±)(trans)-10-[1-[1-(3-Methoxythiophene-2-carbonyl)-4-piperidyl]-4-piperidyl]-8-oxa-10-azabicyclo[4.4.0]decan-9-one;
Ethyl 3-methyl-3-(4-((4aS,8aS)-3-oxo-2H-benzo[b][1,4]oxazin-4(3H,4aH,5H,6H,7H,8H,8aH)-yl)piperidin-1-yl)pyrrolidine-1-carboxylate (ISOMER 1);
Ethyl 3-methyl-3-(4-((4aS,8aS)-3-oxo-2H-benzo[b][1,4]oxazin-4(3H,4aH,5H,6H,7H,8H,8aH)-yl)piperidin-1-yl)pyrrolidine-1-carboxylate (ISOMER 2)
or pharmaceutically acceptable salt thereof.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I to XV The optically active forms of the compound of the invention may be prepared, for example, by chiral chromatographic separation of a racemate, by synthesis from optically active starting materials or by asymmetric synthesis based on the procedures described thereafter.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972), each of which is incorporated herein by reference in their entireties. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

It will also be appreciated that certain compounds of the present invention may exist as geometrical isomers, for example E and Z isomers of alkenes. The present invention includes any geometrical isomer of a compound of Formula I to XV. It will further be understood that the present invention encompasses tautomers of the compounds of the Formula I to XV.

It will also be understood that certain compounds of the present invention may exist in solvated, for example hydrated, as well as unsolvated forms. It will further be understood that the present invention encompasses all such solvated forms of the compounds of the Formula I to XV.

Within the scope of the invention are also salts of the compounds of the Formula I to XV. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of Formula I to XV above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

In some embodiments, the compounds of Formula I to VIII and X to XV are prodrugs. As used herein, “prodrug” refers to a moiety that releases a compound of the invention when administered to a patient. Prodrugs can be prepared by modifying functional groups present in the compounds in such a way that the modifications are cleaved, either in routine manipulation or in vivo, to the parent compounds. Examples of prodrugs include compounds of the invention as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference in their entireties.

### Compositions, Methods and Uses

We have now found that many of the compounds of the invention tested have activity as pharmaceuticals, in particular as agonists of M1 receptors. More particularly, many of the compounds of the invention tested exhibit selective activity as agonist of the M1 receptors and are useful in therapy, especially for relief of various pain conditions such as chronic pain, neuropathic pain, acute pain, cancer pain, pain caused by rheumatoid arthritis, migraine, visceral pain etc. This list should however not be interpreted as exhaustive. Additionally, compounds of the present invention may be useful in other disease states in which dysfunction of M1 receptors is present or implicated. Furthermore, the compounds of the invention may be used to treat cancer, multiple sclerosis, Parkinson's disease, Huntington's chorea, schizophrenia, Alzheimer's disease, anxiety disorders, depression, obesity, gastrointestinal disorders and cardiovascular disorders.

In some embodiments, the compounds may be used to treat schizophrenia or Alzheimer's disease.

In another embodiment, the compounds may be used to treat pain.

In another particular embodiment, the compounds may be used to treat neuropathic pain.

Compounds of the invention may be useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention may be useful in disease states where degeneration or dysfunction of M1 receptors is present or implicated in that paradigm. This may involve the use of isotopically labelled versions of the compounds of the invention in diagnostic techniques and imaging applications such as positron emission tomography (PET).

Compounds of the invention may be useful for the treatment of diarrhea, depression, anxiety and stress-related disorders such as post-traumatic stress disorder, panic disorder, generalized anxiety disorder, social phobia, and obsessive compulsive disorder, urinary incontinence, premature ejaculation, various mental illnesses, cough, lung oedema, various gastrointestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following miocardial infarction, obesity, spinal injury and drug addiction, including the treatment of alcohol, nicotine, opioid and other drug abuse and for disorders of the sympathetic nervous system for example hypertension.

Compounds of the invention may be useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers, and opioids.

A further aspect of the invention is a method for the treatment of a subject suffering from any of the conditions discussed above, whereby an effective amount of a compound according to the Formula I above, is administered to a patient in need of such treatment.

The present invention further provides the use of any of the compounds according to the Formula I above, for the manufacture of a medicament for the treatment of any of the conditions discussed above.

The present invention invention further provides a compound of Formula I, or pharmaceutically acceptable salt or solvate thereof, as hereinbefore defined for use in therapy.

In a further aspect, the present invention provides the use of a compound of Formula I, or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be contrued accordingly. The term “therapy” within the context of the present invention further encompasses to administer an effective amount of a compound of the present invention, to mitigate either a pre-existing disease state, acute or chronic, or a recurring condition. The term “therapy” within the context of the present invention encompasses (a) inhibiting a disease, condition or disorder in an individual who is experiencing or displaying the pathology or symptomatology of the disease, condition or disorder (i.e., arresting further development of the pathology and/or symptomatology); (b) retarding a disease, condition or disorder in an individual who is experiencing or displaying the pathology or symptomatology of the disease, condition or disorder (i.e., slowing down the development of the pathology and/or symptomatology); and (c) ameliorating the disease; for example, ameliorating a disease, condition or disorder in an individual who is experiencing or displaying the pathology or symptomatology of the disease, condition or disorder (i.e., reversing the pathology and/or symptomatology). This definition also encompasses prophylactic therapies for prevention of recurring conditions and continued therapy for chronic disorders.

The phrase “therapeutically effective amount” refers to the amount of a compound of the invention that elicits the biological or medicinal response in a tissue, system, animal, individual, patient, or human that is being sought by a researcher, veterinarian, medical doctor or other clinician. The desired biological or medicinal response may include preventing the disorder in an individual (e.g., preventing the disorder in an individual that may be predisposed to the disorder, but does not yet experience or display the pathology or symptomatology of the disease). The desired biological or medicinal response may also include inhibiting the disorder in an individual that is experiencing or displaying the pathology or symptomatology of the disorder (i.e., arresting or slowing further development of the pathology and/or symptomatology). The desired biological or medicinal response may also include ameliorating the disorder in an individual that is experiencing or displaying the pathology or symptomatology of the disease (i.e., reversing the pathology or symptomatology).

The therapeutically effective amount provided in the treatment of a specific disorder will vary depending the specific disorder(s) being treated, the size, age, and response pattern of the individual the severity of the disorder(s), the judgment of the attending clinician, the manner of administration, and the purpose of the administration, such as prophylaxis or therapy. In general, effective amounts for daily oral administration may be about 0.01 to 1000 mg/kg, 0.01 to 50 mg/kg, about 0.1 to 10 mg/kg and effective amounts for parenteral administration may be about 0.01 to 10 mg/kg, or about 0.1 to 5 mg/kg.

The compounds of the present invention may be useful in therapy, especially for the therapy of various pain conditions including, but not limited to: acute pain, chronic pain, neuropathic pain, back pain, cancer pain, and visceral pain. In a particular embodiment, the compounds may be useful in therapy for neuropathic pain. In an even more particular embodiment, the compounds may be useful in therapy for chronic neuropathic pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, transdermally, intracerebroventricularly and by injection into the joints.

In one embodiment of the invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w/w (percent by weight), more preferably from 0.10 to 50% w/w, of the compound of the invention, all percentages by weight being based on total composition.

Within the scope of the invention is the use of any compound of Formula I as defined above for the manufacture of a medicament.

Also within the scope of the invention is the use of any compound of Formula I for the manufacture of a medicament for the therapy of pain.

Additionally provided is the use of any compound according to Formula I for the manufacture of a medicament for the therapy of various pain conditions including, but not limited to: acute pain, chronic pain, neuropathic pain, back pain, cancer pain, and visceral pain.

A further aspect of the invention is a method for therapy of a subject suffering from any of the conditions discussed above, whereby an effective amount of a compound according to the Formula I above, is administered to a patient in need of such therapy.

Additionally, there is provided a pharmaceutical composition comprising a compound of Formula I or a pharmaceutically acceptable salt thereof, in association with a pharmaceutically acceptable carrier.

Particularly, there is provided a pharmaceutical composition comprising a compound of Formula I or a pharmaceutically acceptable salt thereof, in association with a pharmaceutically acceptable carrier for therapy, more particularly for therapy of pain.

Further, there is provided a pharmaceutical composition comprising a compound of Formula I or a pharmaceutically acceptable salt thereof, in association with a pharmaceutically acceptable carrier use in any of the conditions discussed above.

In a further embodiment, a compound of the present invention, or a pharmaceutical composition or formulation comprising a compound of the present invention may be administered concurrently, simultaneously, sequentially or separately with one or more pharmaceutically active compound(s) selected from the following:

(i) antidepressants such as amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, robalzotan, sertraline, sibutramine, thionisoxetine, tranylcypromaine, trazodone, trimipramine, venlafaxine and equivalents and pharmaceutically active isomer(s) and metabolite(s) thereof;

(ii) atypical antipsychotics including for example quetiapine and pharmaceutically active isomer(s) and metabolite(s) thereof; amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, lithium, loxapine, mesoridazine, olanzapine, paliperidone, perlapine, perphenazine, phenothiazine, phenylbutlypiperidine, pimozide, prochlorperazine, risperidone, quetiapine, sertindole, sulpiride, suproclone, suriclone, thioridazine, trifluoperazine, trimetozine, valproate, valproic acid, zopiclone, zotepine, ziprasidone and equivalents thereof;

(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutlypiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof;

(iv) anxiolytics including for example alnespirone, azapirones,benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof;

(v) anticonvulsants including, for example, carbamazepine, valproate, lamotrogine, gabapentin and equivalents and pharmaceutically active isomer(s) and metabolite(s) thereof;

(vi) Alzheimer's therapies including, for example, donepezil, memantine, tacrine and equivalents and pharmaceutically active isomer(s) and metabolite(s) thereof;

(vii) Parkinson's therapies including, for example, deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof;

(viii) migraine therapies including, for example, almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof;

(ix) stroke therapies including, for example, abciximab, activase, NXY-059, citicoline, crobenetine, desmoteplase,repinotan, traxoprodil and equivalents and pharmaceutically active isomer(s) and metabolite(s) thereof;

(x) over active bladder urinary incontinence therapies including, for example, darafenacin, falvoxate, oxybutynin, propiverine, robalzotan, solifenacin, tolterodine and and equivalents and pharmaceutically active isomer(s) and metabolite(s) thereof;

(xi) neuropathic pain therapies including, for example, gabapentin, lidoderm, pregablin and equivalents and pharmaceutically active isomer(s) and metabolite(s) thereof;

(xii) nociceptive pain therapies such as celecoxib, etoricoxib, lumiracoxib, rofecoxib, valdecoxib, diclofenac, loxoprofen, naproxen, paracetamol and equivalents and pharmaceutically active isomer(s) and metabolite(s) thereof;

(xiii) insomnia therapies including, for example, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos,secobarbital, zaleplon, zolpidem and equivalents and pharmaceutically active isomer(s) and metabolite(s) thereof; and

(xiv) mood stabilizers including, for example, carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Such combinations employ the compounds of this invention within the dosage range described herein and the other pharmaceutically active compound or compounds within approved dosage ranges and/or the dosage described in the publication reference.

In an even further embodiment, a compound of the present invention, or a pharmaceutical composition or formulation comprising a compound of the present invention may be administered concurrently, simultaneously, sequentially or separately with one or more pharmaceutically active compound(s) selected from buprenorphine; dezocine; diacetylmorphine; fentanyl; levomethadyl acetate; meptazinol; morphine; oxycodone; oxymorphone; remifentanil; sufentanil; and tramadol.

In a particular embodiment, it may be particularly effective to administrate a combination containing a compound of the invention and a second active compound selected from buprenorphine; dezocine; diacetylmorphine; fentanyl; levomethadyl acetate; meptazinol; morphine; oxycodone; oxymorphone; remifentanil; sufentanil; and tramadol to treat chronic nociceptive pain. The efficacy of this therapy may be demonstrated using a rat SNL heat hyperalgesia assay described below.

The methods, uses, compounds for use in therapy, and pharmaceutical compositions may utilize any of the embodiments of the compounds of Formulas I to VIII or X to XV, or any combination thereof.

### Syntheses and Processes

The compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

The compounds of present invention can be conveniently prepared in accordance with the procedures outlined in the schemes below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds of the invention.

The processes described herein can be monitored according to any suitable method known in the art. For example, product formation can be monitored by spectroscopic means, such as nuclear magnetic resonance spectroscopy (e.g., ¹H or ¹³C NMR) infrared spectroscopy, spectrophotometry (e.g., UV-visible), or mass spectrometry, or by chromatography such as high performance liquid chromatography (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 4d. Ed., Wiley & Sons, 2007, which is incorporated herein by reference in its entirety. Adjustments to the protecting groups and formation and cleavage methods described herein may be adjusted as necessary in light of the various substituents.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of the present invention may be made by a variety of methods, as described herein. For example, to make compounds of Formula I, wherein Y is —CR³R⁴— and X is —NR⁸—, a BOC (tert-butylcarbamate) hydroxymethylcyclic amine (1) can be reacted to form an azide (4) by converting the hydroxyl group of (1) to a leaving group, followed by treatment with sodium azide and removal of the BOC protecting group, as shown in Scheme I. The amino group of the azide (4) can then be reacted with (5) via reductive amination, followed by conversion of the azide (6) to give the amine (7). The amine (7) can then be cyclized to using a phosgene equivalent such as 1,1′-carbonyldiimidazole (“CDI”) to give compound (8). When R⁸ is other than hydrogen, the R⁸ group may be introduced by reacting (8) with a compound of formula, “R⁸-leaving group”, such as R⁸I. The BOC protecting group can then be removed to give the amine (9). The R² groups of the compounds of the present invention can then be added by converting the amine (9) by various processes such as those shown in Schemes I-A, I-B, I-C, I-D, and I-E, depending on the type of R² group.

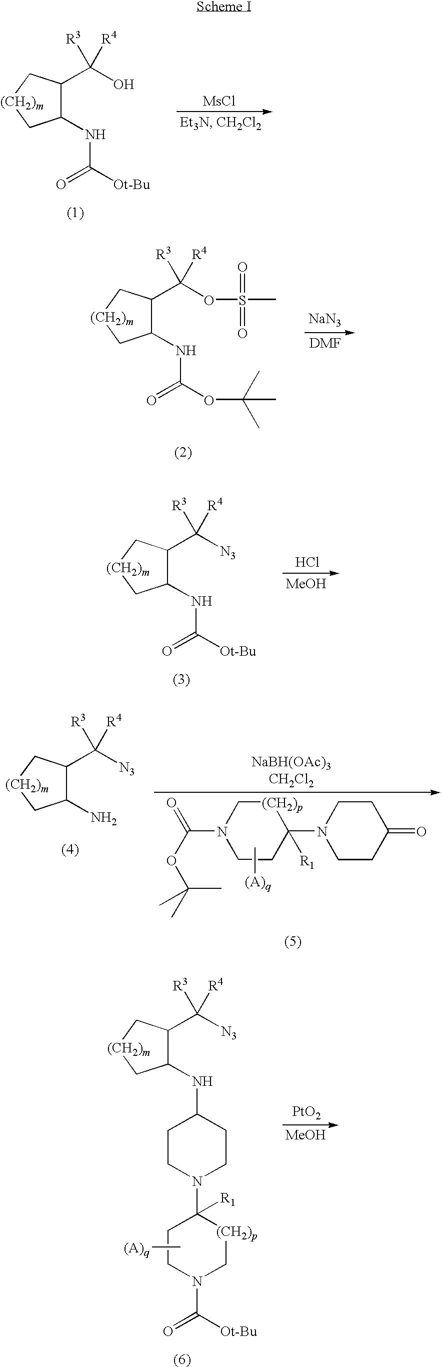

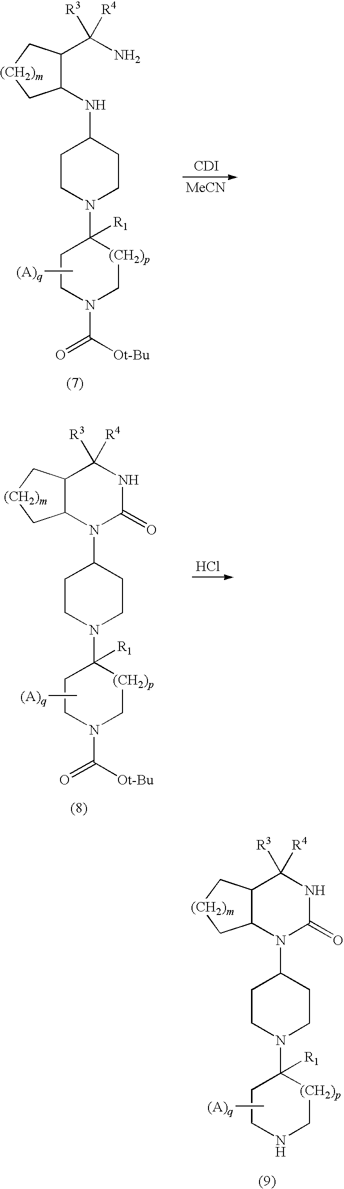

In Scheme I-A, compound (9) may be converted to an amide using an acid halide of formula “R^{b}C(O)-halogen”, such as R^{b}C(O)Cl, generally in the presence of a base such as a tertiary amine (e.g., triethylamine or diisopropylethylamine), imidazole, N,N-dimethyl-4-aminopyridine, or the like. Alternatively, a carboxylic acid of formula R^{b}C(O)OH may be used in the presence of a coupling agent such as HATU, EDC, or equivalent thereof and a base, such a tertiary amine (e.g., triethylamine or diisopropylethylamine), imidazole, N,N-dimethyl-4-aminopyridine, or the like

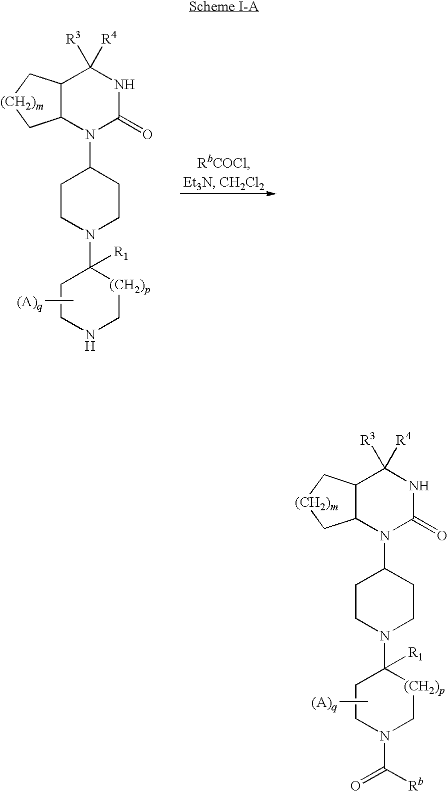

In Scheme I-B, compound (9) may be converted to a carbamate using a compound of formula “R^{a}OC(O)-halogen”, such as R^{a}OC(O)Cl, generally in the presence of a base such as a tertiary amine (e.g., triethylamine or diisopropylethylamine), imidazole, N,N-dimethyl-4-aminopyridine, or the like.

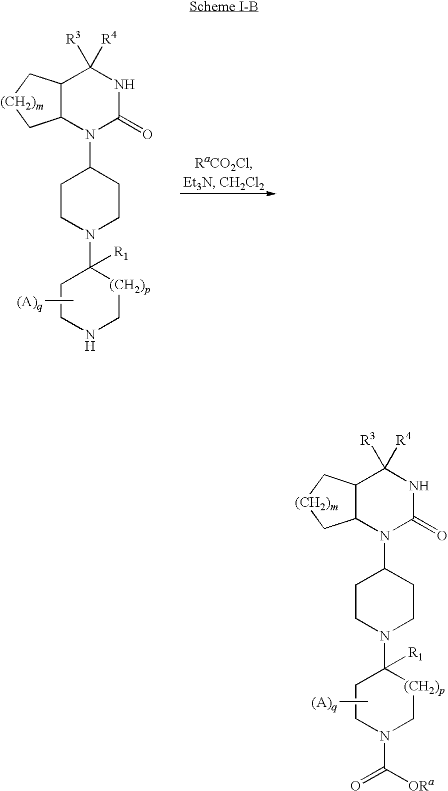

In Scheme I-C, compound (9) may be converted to a urea by first transforming (9) to an ester (R′ is methyl, ethyl or the like), followed by reaction with an amine of formula “HNR^{c}R^{d}”. Alternatively, a urea, wherein R^{d} is hydrogen, may be formed by reacting (9) with an isocyanate of formula “R^{c}—N═C═O”.

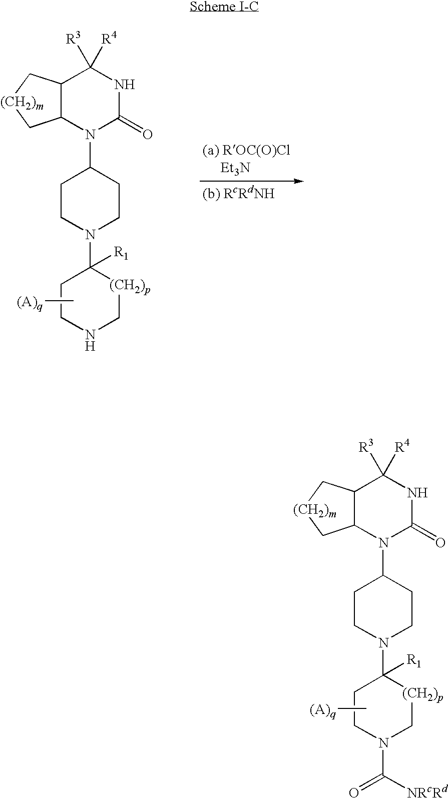

In Scheme I-D, (9) may be reacted with a compound of formula, “R²-LG”, wherein LG is a leaving group such as a tosylate, triflate, or halogen group under appropriate conditions (such as those for alkylation), to form a compound wherein R² is an unsubstituted or substituted C₁₋₆ alkyl, C₂₋₆ alkenyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl-C₁₋₃alkyl, or C₃₋₉heteroaryl-C₁₋₃alkyl group.

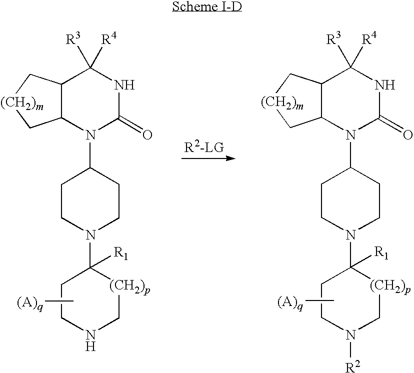

The compound (5) may be prepared by reductive amination of a BOC protected 4-oxopiperidine with a 4-hydroxypiperidine, 3-hydroxypyrrolidine, or 4-hydroxyazepane via methods known in the art. Alternatively, (5) may be prepared by the method shown in Scheme I-E below. In Scheme I-E, an appropriate BOC protected 4-oxopiperidine, 3-oxopyrrolidine, or 4-oxoazepane is reacted with a 4-hydroxypiperidine in the presence of titantium isopropoxide in 1,2-dichloroethane for 18 hours at room temperature. The R¹ group may be added by reacting the product of the previous reaction with diethylaluminum cyanide in tolutene at room temperature for 24 hours to form the cyanate, followed by reaction with a Grignard reagent of formula R¹MgBr in THF and toluene at 0° C. The hydroxyl compound may then be oxidized, e.g., via a Swern oxidation.

Alternatively, compounds of Formula I, wherein Y is —CR³R⁴— and X is —NR⁸— may be formed by the methods shown in Scheme II. For example, the azide (4) can be reacted with a BOC protected 4-oxopiperidine to form the azide (10), followed by reduction of the azide to the amine (11). The amine (11) may be cyclized in the presence of a phosgene equivalent such as 1,1′-carbonyldiimidazole in solvent such as acetonitirile to form (12), followed by removal of the BOC protecting group, such as under acidic conditions, to form (13). When R⁸ is other than hydrogen, the R⁸ group may be introduced by reacting (12) with a compound of formula, R⁸-leaving group (such as R⁸I), followed by removal of the BOC protecting group to form (17). Compounds (13) or (17) may then be reacted with (14) to form the amine (15). The amine (15) can be further reacted to add the R² group by the methods illustrated in Schemes I-A to I-D and the surrounding text.

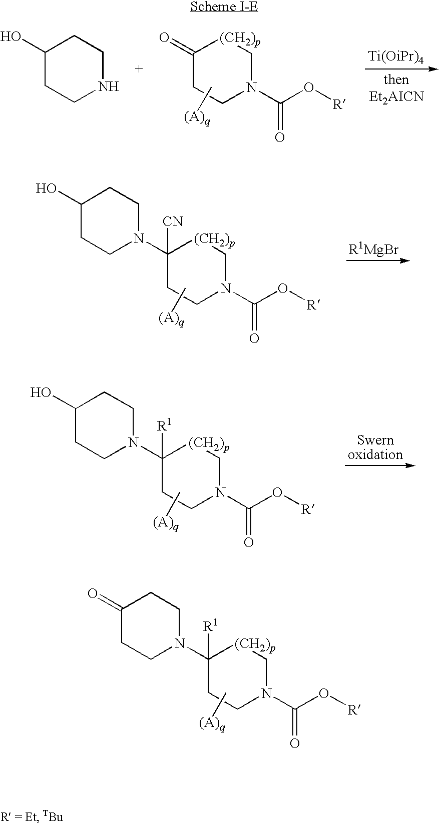

Compounds of Formula I, wherein Y is —O— and X is —CR⁶R⁷— may be formed by the methods shown in Scheme III. For example, compound (18) is formed by benzylating the corresponding hydroxyl compound under standard conditions (Greene's Protective Groups in Organic Synthesis, 4^{th} Ed. (2007). Compound (18) is then reacted with BOC protected 4-oxopiperidine to form (19), followed by removal of the benzyl group to form (20). Compound (20) is then cyclized by reaction with the α-chloroacetyl chloride (21) to form (22), followed by treatment with potassium tert-butoxide in THF to form (23). After removal of the BOC group to form (24), compound (24) is reacted with (25) to form (26), followed by removal of the protecting group, R′, to form the amine (27). The amine (27) may be further reacted to add the R² group by the methods analogous to those illustrated in Schemes I-A to I-D and the surrounding text.

Alternatively, compounds of Formula I, wherein Y is —O— and X is —CR⁶R⁷— may be formed by the methods shown in Schemes IV and IV-A. Compound (18) is then reacted with (5) to form (28), followed by removal of the benzyl group to form (29). Compound (29) is then cyclized by reaction with the α-chloroacetyl chloride, followed by treatment with potassium tert-butoxide in THF to form (30). Compound (30) is then treated to remove the BOC protecting group to form an amine which may be then reacted to add various R² groups, such as EtOC(O)—. Alternatively, after removal of the BOC protecting group, the amine may be reacted to add the R² group by the methods analogous to those illustrated in Schemes I-A to I-D and the surrounding text.

Compounds of Formula I, wherein Y is —S— and X is —CR⁶R⁷— may be formed by the methods analogous to those shown in Scheme III or IV and the surrounding text, except starting from a protected thiol compound. Appropriate protecting groups for thiol groups are summarized in Greene's Protecting Groups in Organic Synthesis, 4^{th} Ed. (2007), chapter 6. Alternatively, the compounds may be synthesized from compounds (20) or (29) of Schemes III and IV by appropriate substitution chemistry. For example, the amine group of (20) or (29) may be first protected. The hydroxyl group of the protected (20) or (29) may then converted to a thiol group by reaction of sodium hydrogen sulfide.

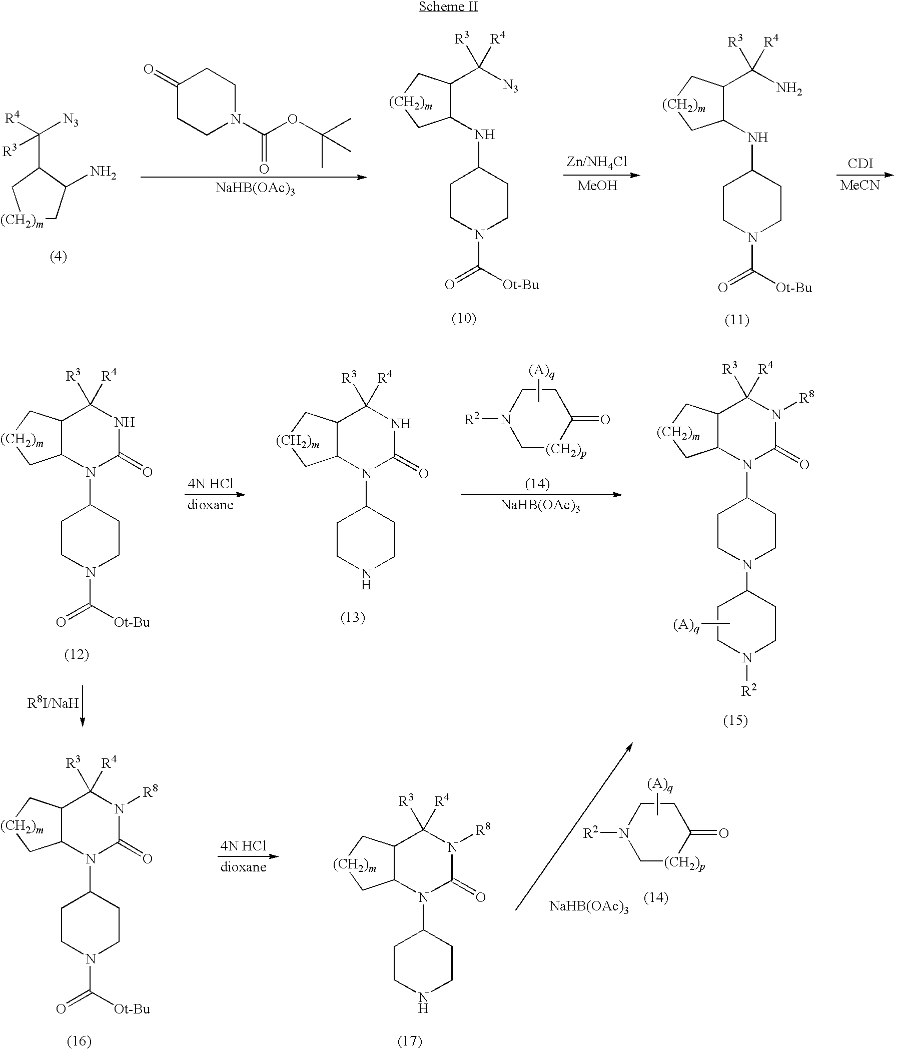

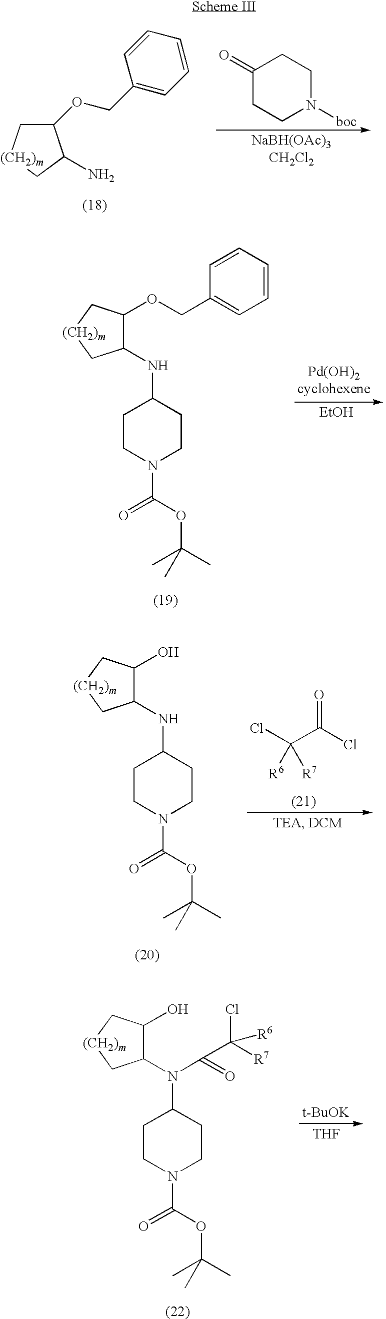

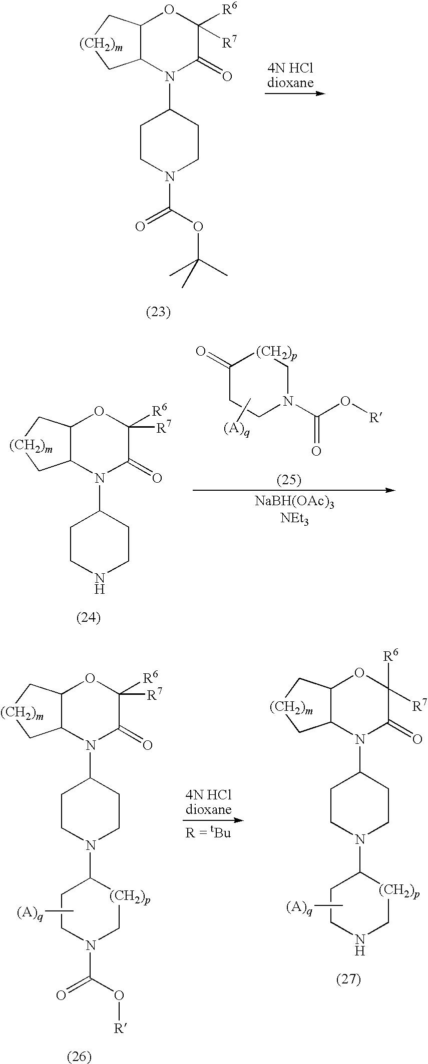

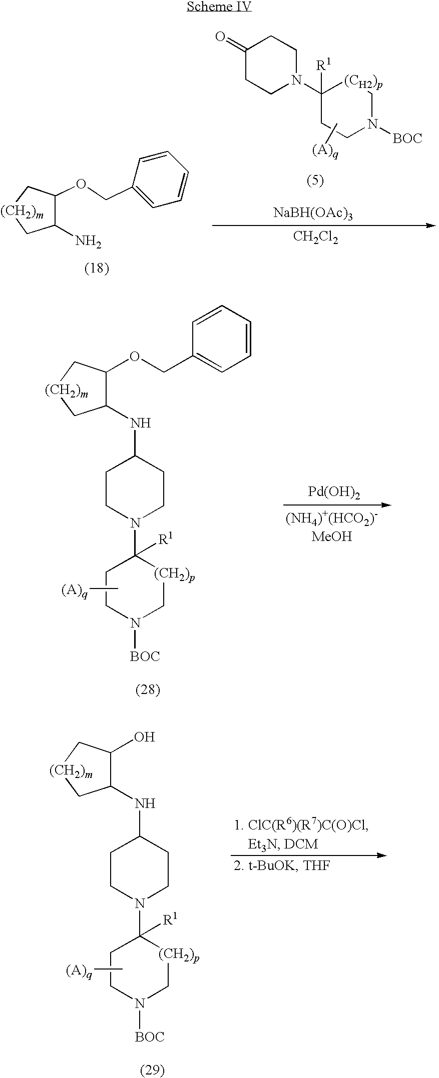

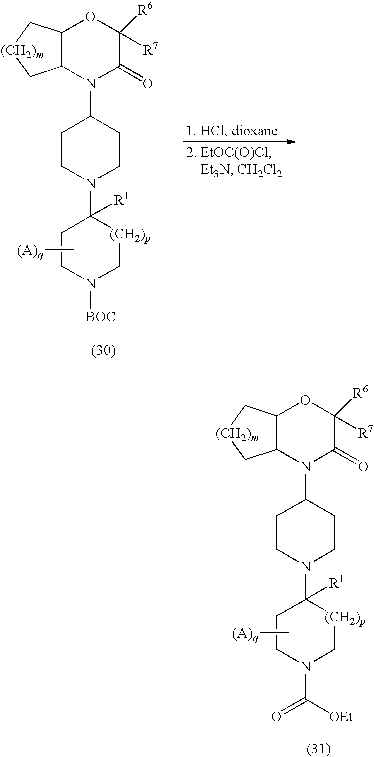

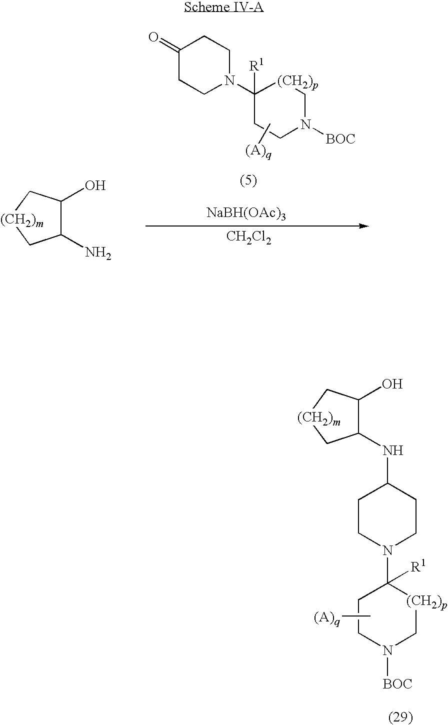

Compounds of Formula I, wherein Y is —CR³R⁴— and X is —O— may be formed by the methods shown in Scheme V. Compound (1) reacted with HCl in methanol to remove the BOC protecting group to form (32). Compound (32) can then be reacted with a BOC protected 4-oxopiperidine to form (33), which may then be cyclized with triphosgene to form (34). After removal of the BOC protecting group to form the amine (35), the amine (35) can be reacted with (36) for form (37), followed by removal of the protecting group, R, to form (38). Compound (38) may then be reacted to add various R² groups, such as R^{a}C(O)—, using the corresponding carboxylic acids in the presence of a coupling agent such as HATU in the presence of a base, such as DIPEA. Alternatively, after removal of the R protecting group, the amine (38) may be reacted to add the R² group by the methods analogous to those illustrated in Schemes I-A to I-D and the surrounding text. Alternatively, compound (32) may be reacted with compound (5) (synthesis illustrated above) instead of a BOC-protected 4-oxopiperidine. The resultant compound may be then be cyclized and deprotected by steps analogous to those illustrated in Scheme V. After removal of the BOC protecting group, R² group may be added by the methods analogous to those illustrated in Schemes I-A to I-D and the surrounding text.

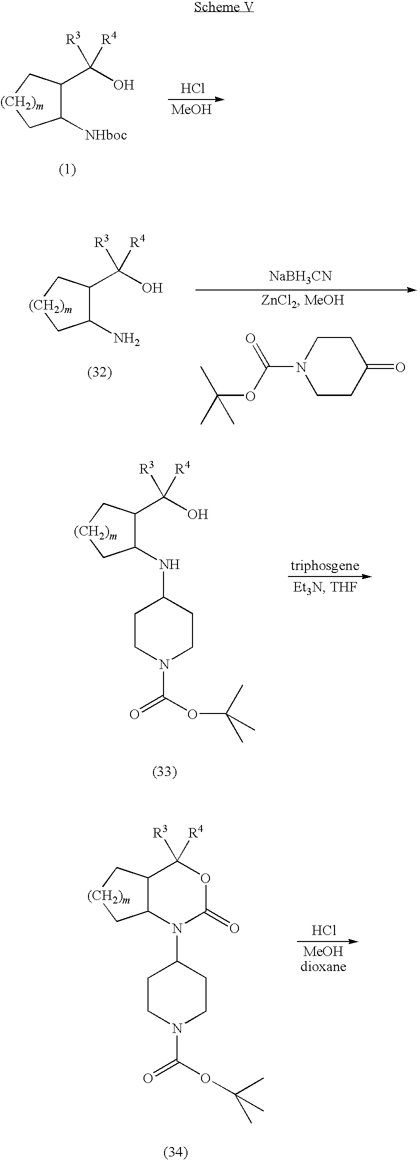

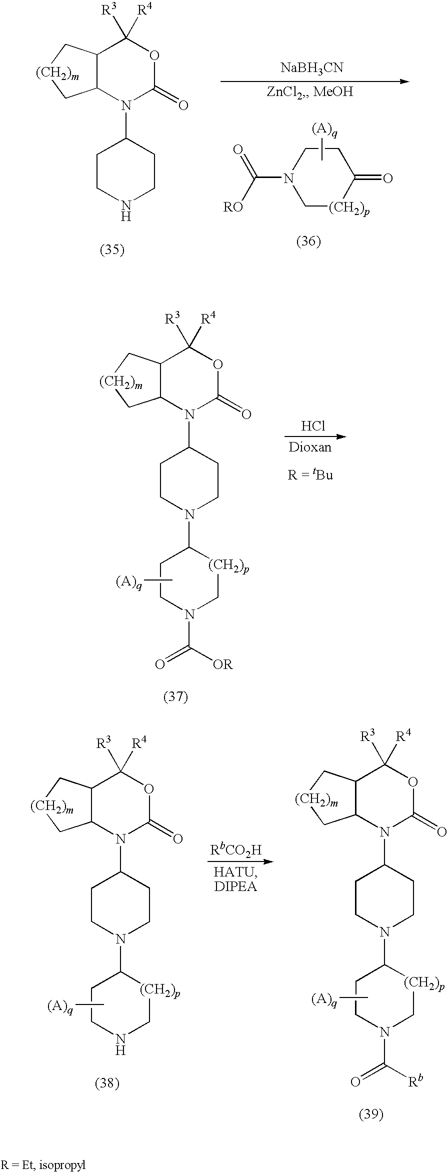

Compounds of Formula I, wherein Y is —S— and X is —CR⁶R⁷— may be formed by methods analogous to those shown in Scheme V and as described in the surrounding text, except starting from a protected thiol compound. Appropriate protecting groups for thiol groups are summarized in Greene's Protecting Groups in Organic Synthesis, 4^{th} Ed. (2007), chapter 6. Alternatively, the compounds may be synthesized from compound (33) of Scheme V by appropriate substitution chemistry. For example, the amine group of (33) may be first protected. The hydroxyl group of the protected (33) may then converted to a thiol group by reaction of sodium hydrogen sulfide.

Compounds of Formula I, wherein Y is —CR³R⁴— and X is —CR⁶R⁷— may be formed by methods shown in Scheme VI. Compound (40) may be reacted to form the nitrile of (41) by conversion of the hydroxyl group to a better leaving group followed by treatment with potassium cyanide. The nitrile (41) may then be reacted with (5) to give (42). The nitrile compound (42) may then be hydrolyzed to convert the nitrile group to a carboxylic acid (43). Compound (43) may then cyclized presence of a coupling reagent (e.g., HATU), a base (e.g., DIPEA, and a suitable organic solvent (e.g., DMF), followed by removal of the BOC protecting group to give the amine (44). The amine (44) may be reacted to add the R² group by the methods analogous to those illustrated in Schemes I-A to I-D and the surrounding text.

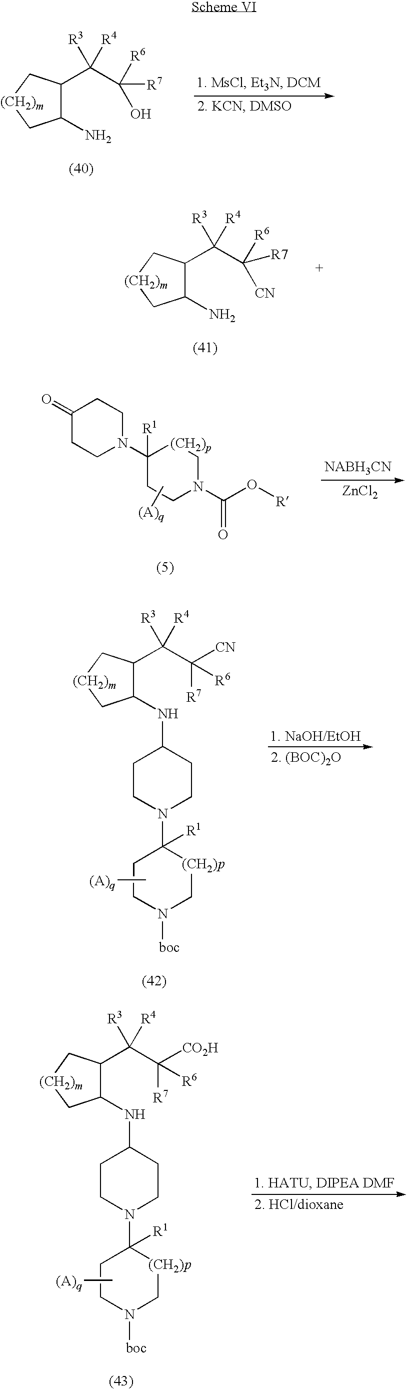

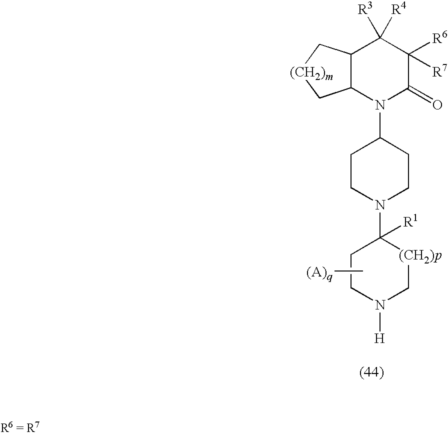

Compound (40) may be made by various methods, such as the method shown in Scheme VII. For example, the hydroxyl group of compound (1) may be converted to a cyano group (e.g., nitrile), for example, by treatment of (1) with mesyl chloride in the presence of triethylamine in dichloromethane, followed by treatment with potassium cyanide in DMSO. The cyano group may then be hydrolyzed to a carboxylic acid using sodium hydroxide in ethanol to give (45). The carboxylic acid (45) may then converted to the acid chloride by reaction with thionyl chloride, followed by reaction with a Gilman reagent of formula (R⁶)₂CuLi to give the ketone. The ketone may then be reacted with a Grignard reagent of formula R⁷MgBr to give the alcohol, followed by removal of the benzyl protecting groups using Pd on carbon and hydrogen to give compound (40). Alternatively, for compounds wherein R⁶ and R⁷ are hydrogen, the carboxylic acid may be converted to an ester (e.g. methyl or ethyl) and then reduced to an alcohol.

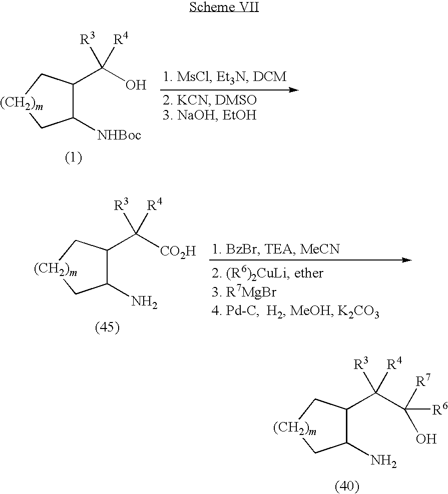

Compounds of Formula I, wherein Y is —NR⁵— and X is —CR⁶R⁷— may be formed by methods shown in Scheme VIII. One of the amine groups of the bisamine (46) may be first protected with a suitable protecting group such as t-butyldimethylsilylether (TBDMS) to form (47). Compound (47) may then reacted with (5) to give (48). Compound (48) may then cyclized by reacting with the α-acetylchloride (21), followed by treatment with tetrabutylammonium fluoride (TBAF) to give (49), followed by selective removal of the TBDMS protecting group to give (50). The BOC protecting group may then be removed under suitable conditions to give the amine (51). Alternatively, compound (50) may be reacted with a reagent of formula R⁵-LG under suitable alkylating conditions (wherein LG is iodide or bromide) to replace the N—H group of compound (50) with R⁵, followed by removal of the BOC protecting group to give an amine. The amine (51) can be further reacted to add the R² group by the methods analogous to those illustrated in Schemes I-A to I-D and the surrounding text. When R⁵ is hydrogen, it may be desirable to protect the amine group of (50) with a protecting group which is stable to conditions which cleave BOC protecting groups, before reacting to add the R² group. The BOC group of (50) may then be removed, followed addition of the R² group, followed by removal of the more stable protecting group. Alternatively, other protecting group methods may be used (for more protecting groups, see Greene, Protecting Groups in Organic Synthesis, 4^{th} Ed. (2007)).

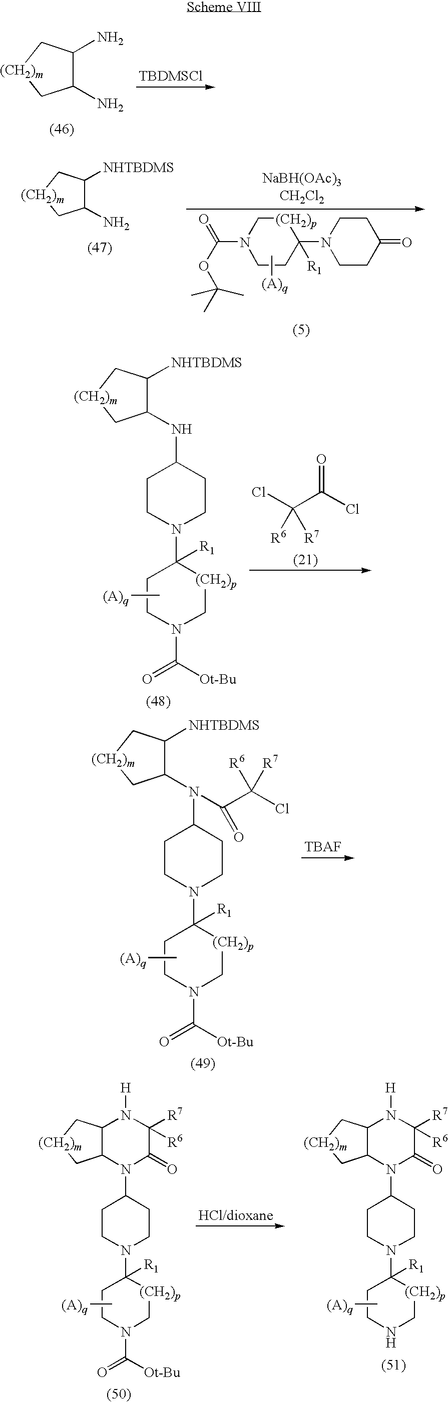

In accordance with the syntheses described above and in the examples, the present invention further provides processes for preparing the compounds of the invention.

In some embodiments, the present invention provides a process for preparing a compound of Formula I, comprising reacting a compound of Formula IX, or pharmaceutically acceptable salt thereof:

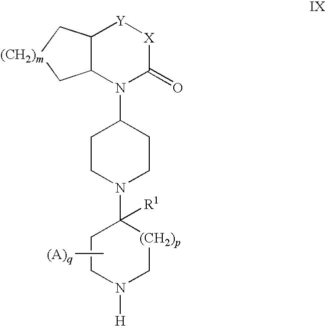

with a compound of Formula R^{a}OC(O)-L¹, or salt thereof, wherein L¹ is halogen, under conditions and for a time sufficient to form a compound of Formula I;
wherein:

Y is —CR³R⁴—, —NR⁵—, —O—, or —S—;

X is —CR⁶R⁷—, —NR⁸—, —O—, or —S—;

with the proviso that either Y is —CR³R⁴— or X is —CR⁶R⁷—;

each A is, independently, C₁₋₃ alkyl;

R¹ is hydrogen, C₁₋₆ alkyl, or C₁₋₆ haloalkyl;

R² is —C(O)OR^{a};

R³, R⁴, R⁶, and R⁷ are each, independently, hydrogen, C₁₋₄ alkyl, or C₁₋₄ haloalkyl;

R⁵ and R⁸ are each, independently, hydrogen, C₁₋₄ alkyl, or C₁₋₄ haloalkyl;

R^{a}, R^{b}, R^{c}, and R^{d} are each, independently, hydrogen, C₁₋₇ alkyl, C₂₋₆ alkenyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein said C₆₋₁₀aryl, C₆₋₁₀aryl-C₁₋₃alkyl, C₃₋₉ heteroaryl, and C₃₋₉ heteroaryl-C₁₋₃alkyl are each optionally substituted by 1, 2, 3, or 4 independently selected R¹² groups; wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl, are each optionally substituted by 1, 2, 3, or 4 independently selected R¹³ groups; and wherein the C₁₋₇ alkyl, C₂₋₆ alkenyl, C₂₋₆ alkynyl, C₁₋₆ haloalkyl, C₁₋₇ alkoxy, and C₁₋₆ haloalkoxy are each optionally substituted by 1, 2, or 3 independently selected R¹⁴ groups;

each R¹², R¹³, and R¹⁴ is, independently, phenyl, C₃₋₆ cycloalkyl, C₂₋₅ heterocycloalkyl, C₃₋₅ heteroaryl, —CN, —SR^{g}, —OR^{g}, —O(CH₂)ᵣ—OR^{g}, R^{g}, —C(O)—R^{g}, —CO₂R^{g}, —SO₂R^{g}, —SO₂NR^{g}R^{h}, halogen, —NO₂, —NR^{g}R^{h}, —(CH₂)ᵣNR^{g}R^{h}, or —C(O)—NR^{g}R^{h};

each R^{e}, R^{f}, R^{g} and R^{h} is, independently hydrogen, C₁₋₆ alkyl, C₂₋₆ alkenyl or C₁₋₆ haloalkyl,

m is 1, 2, or 3;

p is 0, 1, or2;

q is an integer from 0 to [6+(p+2)]; and

r is 1, 2, 3 or 4;

with the proviso that the compound is not isopropyl 4′-methyl-4-((4aS,8aS)-2-oxooctahydroquinoxalin-1(2H)-yl)-1,4′-bipiperidine-1′-carboxylate, or pharmaceutically acceptable salt thereof.

In some embodiments, L² is chloro and the conditions comprise use of a base (such as a tertiary amine, including, but not limited to triethylamine or diisopropylethylamine). In some embodiments, L² is hydroxyl and the conditions comprise use of a coupling agent (such as, but not limited to, 1,1′-carbonyldiimidazole or 1-ethyl-3-(3-dimethyllaminopropyl)carbodiimide hydrochloride “EDC”) and in the presence of a base such as a tertiary amine (e.g., triethylamine or diisopropylethylamine), imidazole, N,N-dimethyl-4-aminopyridine, or the like.

In some embodiments, the present invention further provides a process for preparing a compound of Formula I, comprising reacting a compound of Formula IX, or pharmaceutically acceptable salt thereof:

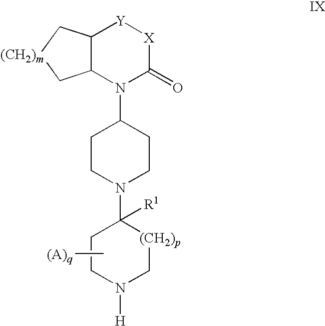

with a compound of Formula R^{b}C(O)-L², or salt thereof, wherein L² is halogen or hydroxyl, under conditions and for a time sufficient to form a compound of Formula I; wherein:

Y is —CR³R⁴—, —NR⁵—, —O—, or —S—;

X is —CR R⁷—, —NR⁸—, —O—, or —S—;

with the proviso that either Y is —CR³R⁴— or X is —CR⁶R⁷—;

each A is, independently, C₁₋₃ alkyl, or two A linked together to form a C₁₋₃alkylene bridge;

R¹ is hydrogen, C₁₋₆ alkyl, or C₁₋₆ haloalkyl;

R² is —C(O)R^{b};

R³, R⁴, R⁶, and R⁷ are each, independently, hydrogen, fluoro, C₁₋₄ alkyl, C₁₋₄ alkoxymethyl, cyanoC₁₋₄ alkyl or C₁₋₄ haloalkyl; R⁵ and R⁸ are each, independently, hydrogen, C₁₋₄ alkyl, or C₁₋₄ haloalkyl;

R^{a}, R^{b}, R^{c}, and R^{d} are each, independently, hydrogen, C₁₋₇ alkyl, C₂₋₆ alkenyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein said C₆₋₁₀aryl, C₆₋₁₀aryl-C₁₋₃alkyl, C₃₋₉ heteroaryl, and C₃₋₉ heteroaryl-C₁₋₃alkyl are each optionally substituted by 1, 2, 3, or 4 independently selected R¹² groups; wherein said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl, are each optionally substituted by 1, 2, 3, or 4 independently selected R¹³ groups; and wherein the C₁₋₇ alkyl, C₂₋₆ alkenyl, C₂₋₆ alkynyl, C₁₋₆ haloalkyl, C₁₋₇ alkoxy, and C₁₋₆ haloalkoxy are each optionally substituted by 1, 2, or 3 independently selected R¹⁴ groups;

each R¹², R¹³, and R¹⁴ is, independently, phenyl, C₃₋₆ cycloalkyl, C₂₋₅ heterocycloalkyl, C₃₋₅ heteroaryl, —CN, —SR^{g}, —OR^{g}, —O(CH₂)ᵣ—OR^{g}, R^{g}, —C(O)—R^{g}, —CO₂R^{g}, —SO₂R^{g}, —SO₂NR^{g}R^{h}, halogen, —NO₂, —NR^{g}R^{h}, —(CH₂)ᵣNR^{g}R^{h}, or —C(O)—NR^{g}R^{h};

each R^{e}, R^{f}, R^{g} and R^{h} is, independently hydrogen, C₁₋₆ alkyl, C₂₋₆ alkenyl or C₁₋₆ haloalkyl,

m is 1, 2, or 3;

p is 0, 1, or2;

q is an integer from 0 to [6+(p+2)]; and

r is 1, 2, 3 or 4;

with the proviso that the compound is not isopropyl 4′-methyl-4-((4aS,8aS)-2-oxooctahydroquinoxalin-1(2H)-yl)-1,4′-bipiperidine-1′-carboxylate, or pharmaceutically acceptable salt thereof.

In some embodiments, L² is chloro. In some embodiments, the conditions comprise use a base such as a tertiary amine (e.g., triethylamine or diisopropylethylamine), imidazole, N,N-dimethyl-4-aminopyridine, or the like. In some embodiments, the conditions further comprise mixing in dichloromethane at about 0° C.

In a further aspect, the present invention provides intermediates useful in the preparation of the compounds of the invention. In some embodiments, the present invention provides a compound of Formula IX:

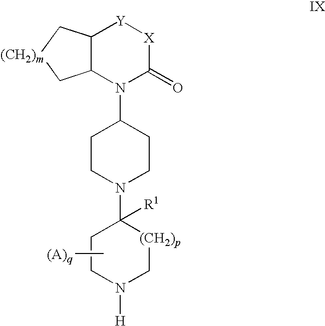

or pharmaceutically acceptable salt thereof;
wherein:

Y is —CR³R⁴—, —NR⁵—, —O—, or —S—;

X is —CR⁶R⁷—, —NR⁸—, —O—, or —S—;

with the proviso that either Y is —CR³R⁴— or X is —CR⁶R⁷—;

each A is, independently, C₁₋₃ alkyl, or two A linked together to form a C₁₋₃alkylene bridge;

R¹ is hydrogen, C₁₋₆ alkyl, or C₁₋₆ haloalkyl;

R³, R⁴, R⁶, and R⁷ are each, independently, hydrogen, fluoro, C₁₋₄ alkyl, C₁₋₄ alkoxymethyl, cyanoC₁₋₄ alkyl or C₁₋₄ haloalkyl; R⁵ and R⁸ are each, independently, hydrogen, C₁₋₄ alkyl, or C₁₋₄ haloalkyl;

m is 1, 2, or 3;

p is 0, 1, or2; and

q is an integer from 0 to [6+(p+2)];

with the proviso that the compound is not isopropyl 4′-methyl-4-((4aS,8aS)-2-oxooctahydroquinoxalin-1(2H)-yl)-1,4′-bipiperidine-1′-carboxylate, or pharmaceutically acceptable salt thereof.

### Biological Evaluation

### Human M1, Rat M1, Human M3 and Human M5 Calcium Mobilization FLIPR™ Assay

The compound activity in the present invention (EC50 or IC50) is measured using a 384 plate-based imaging assay that monitors drug induced intracellular Ca² release in whole cells. Activation of hM1 (human Muscarinic receptor subtype 1, gene bank access NM_{—}000738), rM1 (rat Muscarinic receptor subtype 1, gene bank access NM_{—}080773), hM3 (human Muscarinic receptor subtype 3, gene bank access NM_{—}000740NM_{—}000740) and hM5 (human Muscarinic receptor subtype 5, gene bank access NM_{—}0121258), receptors expressed in CHO cells (Chinese hamster ovary cells, ATCC) is quantified in a Molecular Devices FLIPR IITM instrument as an increase in fluorescent signal. Inhibition of hM3 and hM5 by compounds is determined by the decrease in fluorescent signal in response to 2 nM acetylcholine activation.

CHO cells are plated in 384-well black/clear bottom poly-D-lysine plates (Becton Dickinson, 4663) at 8000 cells/well/50 μl for 24 hours in a humidified incubator (5% CO2 and 37° C.) in DMEM/F12 medium (Wisent 319-075-CL) without selection agent. Prior to experiment, the cell culture medium is removed from the plates by inversion. A loading solution of 25 μl of Hank's balanced salt solution 1× (Wisent 311-506-CL), 10 mM Hepes (Wisent 330-050-EL) and 2.5 mM Probenicid at pH 7.4 (Sigma Aldrich Canada P8761-100 g) with 2 μM calcium indicator dye (FLUO-4AM, Molecular Probes F14202) and Pluronic acid F-127 0.002% (Invitrogen P3000MP) is added to each well. Plates are incubated at 37° C. for 60 minutes prior to start the experiment. The incubation is terminated by washing the cells four times in assay buffer, leaving a residual 25 μl buffer per well. Cell plates are then transferred to the FLIPR, ready for compound additions.

The day of experiment, acetylcholine and compounds are diluted in assay buffer in three-fold concentration range (10 points serial dilution) for addition by FLIPR instrument. For all calcium assays, a baseline reading is taken for 10 seconds followed by the addition of 12.5 μl of compounds, resulting in a total well volume of 37.5 μl. Data is collected every second for 60 pictures and then every 6 seconds for 20 pictures prior to the addition of agonist. For hM3 and hM5, before agonist addition, a second baseline reading is taken for 10 seconds followed by the addition of 12.5 μl of agonist or buffer, producing a final volume of 50 μl. After agonist stimulation, the FLIPR continues to collect data every second for 60 pictures and then every 6 seconds for 20 pictures. The fluorescence emission is read using filter 1 (emission 510-570 nm) by the FLIPR on board CCD camera.

Calcium mobilization output data are calculated as the maximal relative fluorescence unit (RFU) minus the minimal value for both compound and agonist reading frame (except for hM1 and rM1 using only the maximal RFU). Data are analyzed using sigmoidal fits of a non-linear curve-fitting program (XLfit version 4.2.2 Excel add-in version 4.2.2 build 18 math 1Q version 2.1.2 build 18). All pEC50 and pIC50 values are reported as arithmetic means±standard error of mean of ‘n’ independent experiments.

### hM2 Receptor GTPγS Binding

Membranes produced from Chinese hamster ovary cells (CHO) expressing the cloned human M2 receptor (human Muscarinic receptor subtype 2, gene bank access NM_{—}000739), are obtained from Perkin-Elmer (RBHM2M). The membranes are thawed at 37° C., passed 3 times through a 23-gauge blunt-end needle, diluted in the GTPγS binding buffer (50 mM Hepes, 20 mM NaOH, 100 mM NaCl, 1 mM EDTA, 5 mM MgCl₂, pH 7.4, 100 μM DTT). The EC₅₀, IC₅₀ and Eₘₐₓ of the compounds of the invention are evaluated from 10-point dose-response curves (three fold concentration range) done in 60 μl in 384-well non-specific binding surface plate (Corning). Ten microliters from the dose-response curves plate (5× concentration) are transferred to another 384 well plate containing 25 μl of the following: 5 μg of hM2 membranes, 500 μg of Flashblue beads (Perkin-Elmer) and GDP 25 μM. An additional 15 μl containing 3.3× (60,000 dpm) of GTPγ³⁵S (0.4 nM final) are added to the wells resulting in a total well volume of 50 μl. Basal and maximal stimulated [³⁵S]GTPγS binding are determined in absence and presence of 30 μM final of acetylcholine agonist. The membranes/beads mix are pre-incubated for 15 minutes at room temperature with 25 μM GDP prior to distribution in plates (12.5 μM final). The reversal of acetylcholine-induced stimulation (2 μM final) of [³⁵S]GTPγS binding is used to assay the antagonist properties (IC₅₀) of the compounds. The plates are incubated for 60 minutes at room temperature then centrifuged at 400 rpm for 5 minutes. The radioactivity (cpm) is counted in a Trilux (Perkin-Elmer).

Values of EC₅₀, IC₅₀ and Eₘₐₓ are obtained using sigmoidal fits of a non-linear curve-fitting program (XLfit version 4.2.2 Excel add-in version 4.2.2 build 18 math 1Q version 2.1.2 build 18) of percent stimulated [³⁵S]GTPγS binding vs. log(molar ligand). All pEC50 and pIC50 values are reported as arithmetic means±standard error of mean of ‘n’ independent experiments.

### hM4 Receptor GTPγS Binding

Membranes produced from Chinese hamster ovary cells (CHO) expressing the cloned human M4 receptor (human Muscarinic receptor subtype 4, gene bank access NM_{—}000741), are obtained from Perkin-Elmer (RBHM4M). The membranes are thawed at 37° C., passed 3 times through a 23-gauge blunt-end needle, diluted in the GTPγS binding buffer (50 mM Hepes, 20 mM NaOH, 100 mM NaCl, 1 mM EDTA, 5 mM MgCl₂, pH 7.4, 100 μM DTT). The EC₅₀, IC₅₀ and Eₘₐₓ of the compounds of the invention are evaluated from 10-point dose-response curves (three fold concentration range) done in 60 μl in 384-well non-specific binding surface plate (Corning). Ten microliters from the dose-response curves plate (5× concentration) are transferred to another 384 well plate containing 25 μl of the following: 10 μg of hM4 membranes, 500 μg of Flashblue beads (Perkin-Elmer) and GDP 40 μM. An additional 15 μl containing 3.3× (60,000 dpm) of GTPγS (0.4 nM final) are added to the wells resulting in a total well volume of 50 μl. Basal and maximal stimulated [³⁵S]GTPγS binding are determined in absence and presence of 30 μM final of acetylcholine agonist. The membranes/beads mix are pre-incubated for 15 minutes at room temperature with 40 μM GDP prior to distribution in plates (20 μM final). The reversal of acetylcholine-induced stimulation (10 μM final) of [³⁵S]GTPγS binding is used to assay the antagonist properties (IC₅₀) of the compounds. The plates are incubated for 60 minutes at room temperature then centrifuged at 400 rpm for 5 minutes. The radioactivity (cpm) is counted in a Trilux (Perkin-Elmer).

Values of EC₅₀, IC₅₀ and Eₘₐₓ are obtained using sigmoidal fits of a non-linear curve-fitting program (XLfit version 4.2.2 Excel add-in version 4.2.2 build 18 math 1Q version 2.1.2 build 18) of percent stimulated [³⁵S]GTPγS binding vs. log(molar ligand). All pEC50 and pIC50 values are reported as arithmetic means±standard error of mean of ‘n’ independent experiments.

Certain biological properties of certain compounds of the invention measured using one or more assays described above are listed in Table 1 below.

TABLE 1
Certain Biological Properties of the Certain
Compounds of the Invention.

| | | | hM3 | hM4 | hM5 |
|---|---|---|---|---|---|
| | hM1 EC50 | hM2 EC50 | EC50 | EC50 | EC50 |
| Example No | (nM) | (nM) | (nM) | (nM) | (nM) |
| Example 1 | 1.6 | 380 | | 1700 | |
| Example 2 | 5.3 | >1200 | >49000 | >20000 | >49200 |
| Example 3 | 13 | >12000 | >49000 | >30000 | >31100 |
| Example 4 | 25 | >30000 | >49000 | >30000 | >49200 |
| Example 5 | 31 | | | | |
| Example 6 | 94 | >30000 | >40000 | >30000 | >40000 |
| Example 7 | 4.5 | 70 | 690 | >1600 | 168 |
| Example 8 | <11 | >30000 | >40000 | >30000 | >40000 |
| Example 9 | 17 | 2500 | >40000 | 4300 | 97.7 |
| Example 10 | 22 | >6600 | >40000 | >30000 | >40000 |
| Example 11 | 170 | >30000 | >40000 | >30000 | >40000 |
| Example 12 | 340 | >30000 | >40000 | >30000 | >40000 |
| Example 13 | 200 | >30000 | >40000 | >30000 | >40000 |
| Example 14 | 130 | >30000 | >40000 | >30000 | >40000 |
| Example 15 | 83 | >12000 | >40000 | >30000 | >40000 |
| Example 16 | 220 | | | | |
| Example 17 | 270 | | >40000 | | >40000 |
| Example 18 | 23 | 3700 | >40000 | 8965 | >40000 |
| Example 19 | 46 | >40000 | >40000 | >30000 | >40000 |

In addition, the following compounds are tested in the above assays and it is found that these particular compounds have hM1 EC50 values greater than 2894 nM. These particular compounds are:

isopropyl 4-[4-[(4aS,8aS)-2-oxo-3,4,4a,5,6,7,8,8a-octahydroquinoxalin-1-yl]-1-piperidyl]-4-methyl-piperidine-1-carboxylate;

isopropyl (3S)-3-[4-[(4aS,8aS)-3-oxo-4a,5,6,7,8, 8a-hexahydrobenzo[b][1,4]oxazin-4-yl]-1-piperidyl]pyrrolidine-1-carboxylate;

tert-butyl 4-[4-[(4aR,8aR)-2-oxo-4a,5,6,7,8,8a-hexahydro-4H-benzo[d][1,3]oxazin-1-yl]-1-piperidyl]piperidine-1-carboxylate;

isopropyl 4-[4-[(4aS, 8aS)-3-oxo-4a,5,6,7, 8,8a-hexahydrobenzo[b][1,4]oxazin-4-yl]-1-piperidyl]-4-methyl-piperidine-1-carboxylate;

(4aS,8aS)-1-[1-[1-(2-methylbenzoyl)-4-piperidyl]-4-piperidyl]-4a,5,6,7,8,8a-hexahydro-4H-benzo[d][1,3]oxazin-2-one;

tert-butyl 4-[4-[(4aS,8aS)-3-oxo-4a,5,6,7,8,8a-hexahydrobenzo[b][1,4]oxazin-4-yl]-1-piperidyl]piperidine-1-carboxylate; and

methyl 4-[4-[(4aS,8aS)-2-oxo-4a,5,6,7,8,8a-hexahydro-4H-benzo[d][1,3]oxazin-1-yl]-1-piperidyl]piperidine-1-carboxylate.

### Rat SNL Heat Hyperalgesia Assay

Rats undergo spinal nerve ligation surgery as described in Kim and Chung (1992) (reference 1). Briefly, rats are anesthetized with isoflurane, the left L5 and L6 are isolated and tightly ligated with 4-0 silk thread. The wound is closed by suturing and applying tissue adhesive. Compound testing is performed at day 9 to day 36 post-surgery.

For behavioral testing, the animals are acclimatized to the test room environment for a minimum of 30 min. In order to assess the degree of hyperalgesia, the animals are placed on a glass surface (maintained at 30° C.), and a heat-source is focused onto the plantar surface of the left paw. The time from the initiation of the heat until the animal withdraws the paw is recorded. Each animal is tested twice (with an interval of 10 min between the two tests). A decrease in Paw Withdrawal Latency (PWL, average of the two tests) relative to naive animals indicates a hyperalgesic state. The rats with a PWL of at least 2 seconds less than average PWL of Naive group are selected for compound testing.

Each individual experiment consists of several groups of SNL rats, one group receiving vehicle while the other groups receive different doses of the test article. In all experiments, animals are tested for heat hyperalgesia using the plantar test before drug or vehicle administration to ensure stable heat-hyperalgesia baseline and rats are evenly divided into groups for compound testing. At a suitable interval after vehicle or drug administration, another test is performed to measure PWL. Generally, results from 2 individual experiments are pooled together and the data are presented as the mean paw withdrawal latency (PWL) (s)±standard error of mean (SEM).

A combination containing a compound of the present invention and morphine at a predetermined ratio (e.g., 0.64:1) may be tested using this instant model. The combination drugs may be administered to the rats subcutaneously, orally or combination thereof, simultaneously or sequentially. The results (expressed as ED₅₀) for the combination may be compared with results obtained singly for the compound of the instant invention and morphine at the same or similar dosage range. If the ED₅₀ of the combination is significantly lower than the theoretical ED₅₀ calculated based on the ED₅₀ measured using the compound of the invention and morphine singly, then a synergy for the combination is indicated.

### EXAMPLES

In order that the invention disclosed herein may be more efficiently understood, examples are provided below. It should be understood that these examples are for illustrative purposes only and are not to be construed as limiting the invention in any manner.

The following abbreviations are used herein: “RT” or “rt” means room temperature.

“Preparative LC/MS (high pH)” means high pressure liquid chromatography with mass detection in preparative scale. Conditions used—Column: Waters X-Bridge Prep C18 OBD, 30×50 mm, 5 mm particle size, Mobile phase: A=Water 10 mM NH₄HCO₃ (pH 10) and B: MeCN.

“HATU” means O-(7-Azabenzotriazol-1-yl)-N,N,N′,N′-tetramethyluronium hexafluorophosphate.

“CDI” means 1,1′-Carbonyldiimidazole.

“DIPEA” means Diisopropylethylamine.

Lexichem v1.4 IUPAC namimg software was used to name all the compounds

### Example 1

### Ethyl 4-[4-[(4aR,8aS)-2-oxo-3,4,4a,5,6,7,8,8a-octahydroquinazolin-1-y]-1-piperidyl]piperidine-1-carboxylate

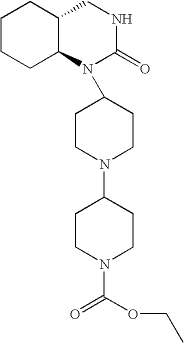

### Step A. The preparation of tert-butyl N-[(1S,2S)-2-(methylsulfonyloxymethyl)cyclohexyl]carbamate

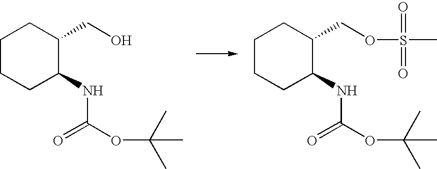

A solution of tert-butyl [(1S,2S)-2-(hydroxymethyl)cyclohexyl]carbamate (10 g, 43.67 mmol) in dichloromethane (50 mL) was added with methanesufonyl chloride (4 mL, 52 mmol) dropwise at 0° C. Triethylamine (7.35 mL, 52 mmol) was then added and the mixture was stirred for 1 hour at room temperature. The reaction was quenched with ice and diluted with dichloromethane. The organic phase was washed with saturated aqueous solution of NaHCO₃ and then with brine, dried and the solvent was removed in vacuo to provide the title compound as a brown solid (15 g). MS (M+1): 308.16.

### Step B. The preparation of tert-butyl N-[(1S,2R)-2-(azidomethyl)cyclohexyl]carbamate

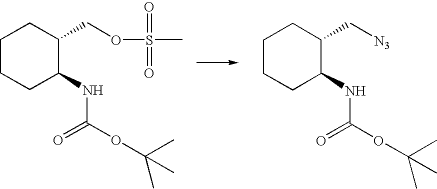

A solution of tert-butyl N-[(1S,2S)-2-(methylsulfonyloxymethyl)cyclohexyl]carbamate (3 g, 9.76 mmol) in DMF (25 mL) was added with sodium azide (1.27 g, 19.54 mmol). The mixture was heated at 120° C. for 3 hours, allowed to cool to room temperature and then quenched with ice. The solvent was removed in vacuo. The residue was dissolved in ethyl acetate (100 mL) and washed with 1N NaOH (10 mL). The organic phase was then dried and concentrated in vacuo to give the title compound (2.48 g), which was used for the next step without any purification. MS (M+1): 255.21.

### Step C. The preparation of (1S,2R)-2-(azidomethyl)cyclohexan-1-amine

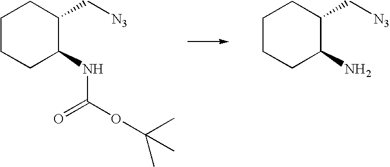

A solution of tert-butyl N-[(1S,2R)-2-(azidomethyl)cyclohexyl]carbamate (2.482 g, 9.76 mmol) in MeOH (20 mL) was added with a solution of 4M HCl in dioxane (15 mL). The reaction mixture was stirred at room temperature over night. The solvents were removed in vacuo to give the title compound (2.2 g), which was used for the next step without further purification.

### Step D. The preparation of tert-butyl 4-[4-[[(1S,2R)-2-(azidomethyl)cyclohexyl]amino]-1-piperidyl]piperidine-1-carboxylate

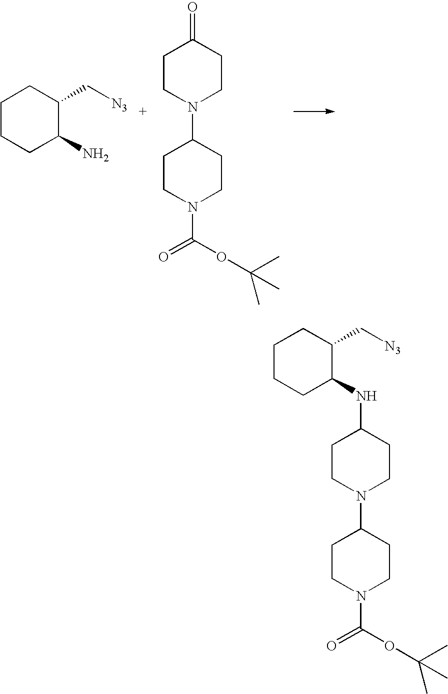

A solution of (1S,2R)-2-(azidomethyl)cyclohexan-1-amine (HCl salt, 2.2 g, 11.55 mmol) in MeOH (20 mL) was added with tert-butyl 4-(4-oxo-1-piperidyl)piperidine-1-carboxylate (2.75 g, 13.82 mmol) followed by sodium triacetoxy borohydride (3 g, 14.15 mmol). The reaction mixture was stirred at room temperature overnight, quenched with 1N NaOH and then diluted with dichloromethane. Phases were separated and aqueous phase was extracted several times with dichloromethane. The combined organic phases were dried and concentrated in vacuo to provide the title compound (2 g), which was used for the next step without any purification. MS (M+1): 421.32.

### Step E. The preparation of tert-butyl 4-[4-[[(1S,2R)-2-(aminomethyl)cyclohexyl]amino]-1-piperidyl]piperidine-1-carboxylate

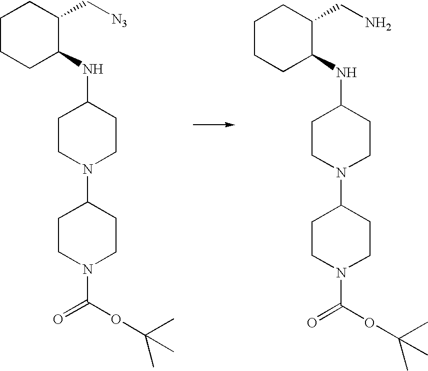

A solution of tert-butyl 4-[4-[[(1S,2R)-2-(azidomethyl)cyclohexyl]amino]-1-piperidyl]piperidine-1-carboxylate (2 g) in EtOH (30 mL) was added with platinum(IV) oxide (200 mg). The reaction mixture was stirred under hydrogen atmosphere (45 psi) at room temperature for 48 hours. The catalyst was filtered off and the filtrate was concentrated in vacuo to provide the title compound as a brown solid (1.6 g), which was used for the next step without any further purification. MS (M+1): 395.37.

### Step F. The preparation of tert-butyl 4-[4-[(4aR,8aS)-2-oxo-3,4,4a,5,6,7,8,8a-octahydroquinazolin-1-yl]-1-piperidyl]piperidine-1-carboxylate

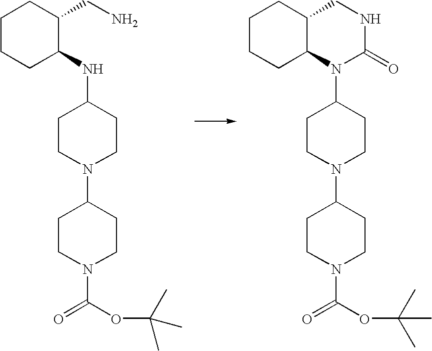

A solution of tert-butyl 4-[4-[[(1S,2R)-2-(aminomethyl)cyclohexyl]amino]-1-piperidyl]piperidine-1-carboxylate (1.6 g, 4.05 mmol) in acetonitrile (50 mL) was added with 1,1-carbonyldiimidazole (0.66 g, 4.05 mmol). The reaction mixture was stirred at room temperature for 3 hours. The solvent was removed in vacuo. The residue was dissolved in dichloromethane and washed with 1N NaOH. The aqueous phase was separated and extracted with dichloromethane. The combined organic phases were dried and the solvent was removed in vacuo to provide the title compound (1.6 g). MS (M+1): 421.33.

### Step G. The preparation of (4aR,8aS)-1-[1-(4-piperidyl)-4-piperidyl]-3,4,4a,5,6,7,8,8a-octahydroquinazolin-2-one

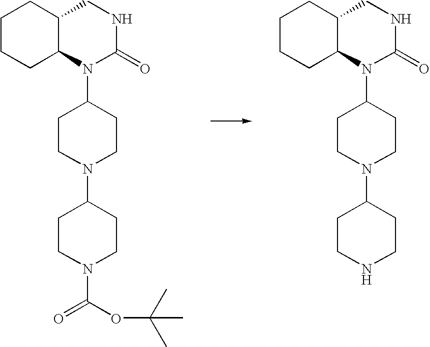

A solution of tert-butyl 4-[4-[(4aR,8aS)-2-oxo-3,4,4a,5,6,7,8,8a-octahydroquinazolin-1-yl]-1-piperidyl]piperidine-1-carboxylate (1.6 g) in MeOH (40 mL) was added with a solution of 4M HCl in dioxane (10 mL, 40.00 mmol). The reaction mixture was stirred at room temperature over night. The solvent was removed in vacuo to provide the title compound as its HCl salt, which was used for the next step without further purification. MS (M+1): 321.38.

### Step H. The preparation of ethyl 4-[4-[(4aR,8aS)-2-oxo-3,4,4a,5,6,7,8,8a-octahydroquinazolin-1-yl]-1-piperidyl]piperidine-1-carboxylate

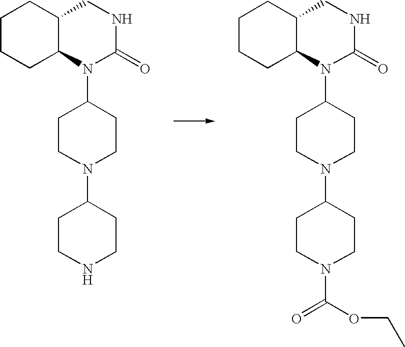

A solution of (4aR,8aS)-1-[1-(4-piperidyl)-4-piperidyl]-3,4,4a,5,6,7,8,8a-octahydroquinazolin-2-one (HCl salt, 0.09 g, 0.28 mmol) in dichloromethane (5 mL) was added with triethylamine (0.11 mL, 0.81 mmol) followed by ethyl chloroformate (0.027 mL, 0.28 mmol) at 0° C. The reaction mixture was stirred at room temperature for 2 hours. The reaction was quenched with ice, diluted with dichloromethane and washed with 1N NaOH. The organic phase was separated and aqueous phase was extracted with dichloromethane. The combined organic phases were dried and the solvent was removed in vacuo. The residue was then purified by preparative LC/MS (high pH) (30-50% MeCN in water) to provide the title compound as a white solid (69 mg, 63%). 1H NMR (400 MHz, CHLOROFORM-D) δ ppm 0.87-1.08 (m, 1 H), 1.09-1.30 (m, 5H), 1.29-1.47 (m, 2H), 1.52-1.87 (m, 8H), 2.09-2.47 (m, 6H), 2.54-3.02 (m, 8H), 3.54-3.71 (m, 1H), 4.06 (q, J=7.03 Hz, 2H), 4.10-4.25 (m, 2H), 5.06-5.28 (m, 1H). MS (M+1): 393.37.

### Example 2

### Propan-2-yl 4-[4-[(4aR,8aS)-2-oxo-3,4,4a,5,6,7,8,8a-octahydroquinazolin-1-yl]-1-piperidyl]piperidine-1-carboxylate

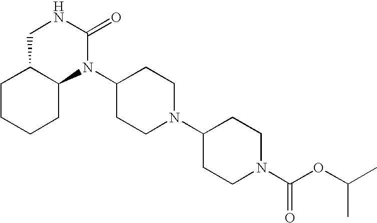

### Step A. The preparation of tert-butyl 4-[[(1S,2R)-2-(azidomethyl)cyclohexyl]amino]piperidine-1-carboxylate

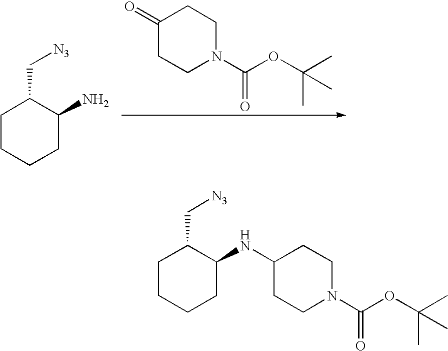

Following the analogous procedure described in step D of the example 1, the title compound was prepared from (1S,2R)-2-(azidomethyl)cyclohexan-1-amine (HCl salt, 7.53 mmol) and tert-butyl 4-oxo-piperidine-1-carboxylate (7.53 mmol). The crude product (2.48 g, 98%) was used for the next step without further purification. MS (M+1): 338.3.

### Step B. The preparation of tert-butyl 4-[4-[[(1S,2R)-2-(aminomethyl)cyclohexyl]amino]-1-piperidyl]piperidine-1-carboxylate

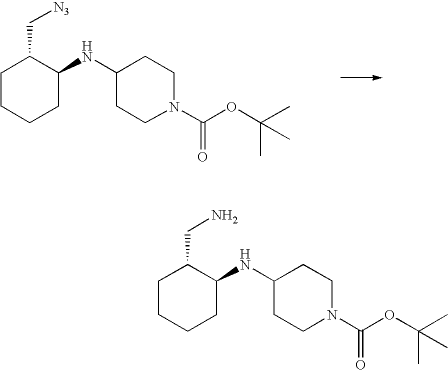

A solution of tert-butyl 4-[4-[[(1S,2R)-2-(azidomethyl)cyclohexyl]amino]-1-piperidyl]piperidine-1-carboxylate (5.0 mmol) in MeOH (25 mL) was added with Zn powder (6.5 g, 100 mmol) followed by NH₄Cl (1.36 g, 25 mmol). The reaction mixture was stirred at room temperature for 3 hours. Filtered through Celite and the filtrate was concentrated in vacuo to give the title compound, which was used for the next step without further purification. MS (M+1): 312.3.

### Step C. The preparation of tert-butyl 4-[4-[(4aR,8aS)-2-oxo-3,4,4a,5,6,7,8,8a-octahydroquinazolin-1-yl]-1-piperidyl]piperidine-1-carboxylate

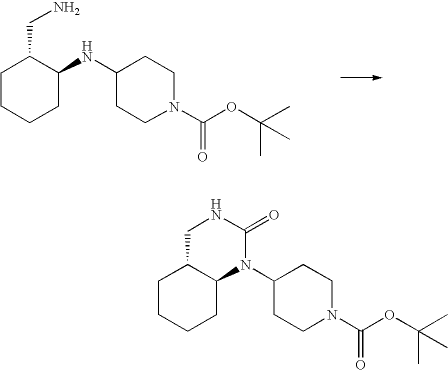

A solution of tert-butyl 4-[4-[[(1S,2R)-2-(aminomethyl)cyclohexyl]amino]-1-piperidyl]piperidine-1-carboxylate (5 mmol) in MeCN (10 mL) was added with 1,1′-carbonyldiimidazole (1.22 g, 7.5 mmol). The reaction mixture was stirred at room temperature for 12 hours. The solvent was removed in vacuo. Water (10 mL) was added to the residue followed by dichloromethane (80 mL). The phases were separated and the aqueous phase was extracted with dichloromethane (2×20 mL). The combined organic phases were washed with brine, dried over Na₂SO₄ and filtered. The solvent was removed in vacuo and the residue was purified by preparative LC/MS (high pH) to give the title compound as white solid (648 mg, 38% over two steps). MS (M+1): 338.2.

### Step D. The preparation of (4aR,8aS)-1-[1-(4-piperidyl)-4-piperidyl]-3,4,4a,5,6,7,8,8a-octahydroquinazolin-2-one

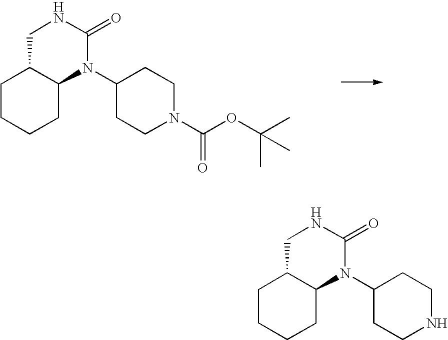

A solution of tert-butyl 4-[4-[(4aR,8aS)-2-oxo-3,4,4a,5,6,7,8,8a-octahydroquinazolin-1-yl]-1-piperidyl]piperidine-1-carboxylate (421 mg, 1.25 mmol) in 4N HCl in dioxane (5 mL) was stirred at room temperature for 3 hours. The solvents were removed in vacuo to give the title compound (338 mg, 99%), which was used in the next step without further purification. MS (M+1): 238.2.

### Step E. The preparation of propan-2-yl 4-[4-[(4aR,8aS)-2-oxo-3,4,4a,5,6,7,8,8a-octahydroquinazolin-1-yl]-1-piperidyl]piperidine-1-carboxylate

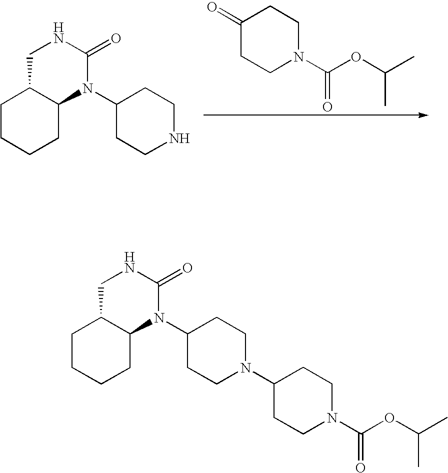

A solution of(4aR,8aS)-1-(4-piperidyl)-3,4,4a,5,6,7,8,8a-octahydroquinazolin-2-one (HCl salt, 0.2 mmol) in dichloromethane (5 mL) was added with triethyl amine (0.2 mmol) followed by isopropyl 4-oxopiperidine-1-carboxylate (37 mg, 0.2 mmol). Sodium triacetoxyborohydride (63 mg, 0.3 mmol) was then added and the reaction mixture was stirred at room temperature for 12 hours. Another portion of isopropyl 4-oxopiperidine-1-carboxylate (18.5 mg, 0.1 mmol) was added followed by catalytic amount of HOAc and stirred at room temperature for another 48 hours. Saturated NaHCO₃ (10 mL) and dichloromethane (20 mL) was added, the phases were separated and the aqueous phase was extracted with dichloromethane (2×10 mL). The combined organic phases were washed with brine, dried over Na₂SO₄ and filtered. The solvents were removed in vacuo. The residue was purified by preparative LC/MS (high pH) to give the title compound as white solid (63 mg, 77% over two steps). 1 H NMR (400 MHz, METHANOL-D4) δ ppm 1.00-1.18 (m, 2H), 1.21 (d, J=6.25 Hz, 6H), 1.27-1.42 (m, 4H), 1.51-1.68 (m, 3H), 1.69-1.78 (m, 2H), 1.80-1.92 (m, 3H), 2.19-2.31 (m, 2H), 2.33-2.53 (m, 4H), 2.64-2.80 (m, 2H), 2.83 (t, J=12.12 Hz, 1H), 2.90-3.06 (m, 4H), 3.45-359 (m, 1H), 4.14 (d, J=12.12 Hz, 2H), 4.77-4.85 (m, 1H). MS (M+1): 407.0.

### Example 3

### (4aR,8aS)-1-[1-[1-(cyclopropanecarbonyl)-4-piperidyl]-4-piperidyl]-3,4,4a,5,6,7,8,8a-octahydroquinazolin-2-one

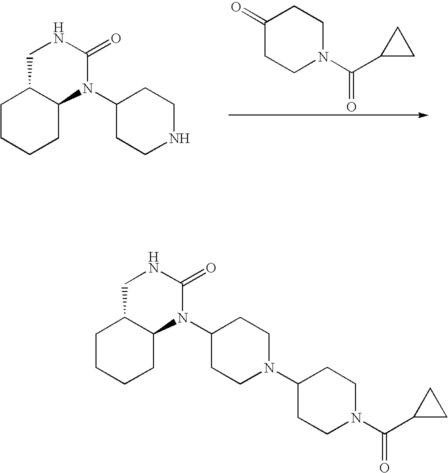

Following the analogous procedure described in step E of the example 2, the title compound was prepared from (4aR,8aS)-1-(4-piperidyl)-3,4,4a,5,6,7,8,8a-octahydroquinazolin-2-one (HCl salt, 0.2 mmol) and 1-(cyclopropylcarbonyl)piperidin-4-one (34 mg, 0.2 mmol). The crude product was purified by preparative LC/MS (high pH) to give the title compound as white solid (22 mg, 28% over two steps). 1H NMR (400 MHz, METHANOL-D4) δ ppm 0.64-0.79 (m, 4H), 0.96-1.18 (m, 2H), 1.21-1.45 (m, 4H), 1.46-1.61 (m, 3H), 1.61-1.71 (m, 2H), 1.73-1.94 (m, 4H), 2.11-2.27 (m, 3H), 2.28-2.39 (m, 2H), 2.44-2.57 (m, 2H), 2.76 (t, J=11.52 Hz, 1H), 2.85-2.99 (m, 4H), 3.03 (t, J=12.70 Hz, 1H), 3.38-3.52 (m, 1H), 4.30 (d, J=13.67 Hz, 1H), 4.46 (d, J=12.89 Hz, 1H). MS (M+1): 389.0.

### Example 4

### (4aR,8aS)-1-[1-[1-(2-methylbenzoyl)-4-piperidyl]-4-piperidyl]-3,4,4a,5,6,7,8,8a-octahydroquinazolin-2-one

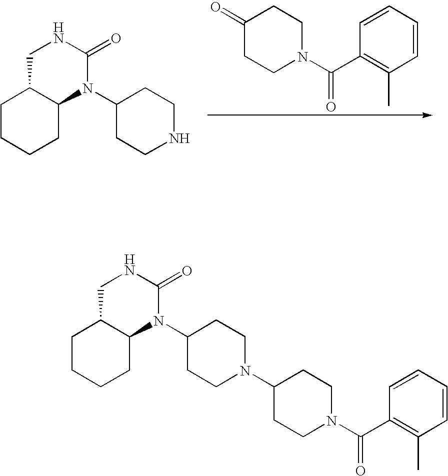

Following the analogous procedure described in step E of the example 2, the title compound was prepared from (4aR,8aS)-1-(4-piperidyl)-3,4,4a,5,6,7,8,8a-octahydroquinazolin-2-one (HCl salt, 0.2 mmol) and 1-(2-methylbenzoyl)piperidin-4-one (44 mg, 0.2 mmol). The crude product was purified by preparative LC/MS (high pH) to give the title compound as white solid (64 mg, 73%).1H NMR (400 MHz, METHANOL-D4) δ ppm 0.96-1.17 (m, 2H), 1.21-1.33 (m, 3H), 1.36-1.46 (m, 1H), 1.48-1.60 (m, 3H), 1.62-1.79 (m, 4H), 1.89-1.98 (m, 1H), 2.14 (s, 3H), 2.17-2.25 (m, 3H), 2.28-2.38 (m, 2H), 2.45-2.57 (m, 1H), 2.70-2.80 (m, 2H), 2.84-3.01 (m, 5H), 3.34-3.53 (m, 2H), 4.65 (d, J=12.50 Hz, 1H), 6.95-7.36 (m, 4H), MS (M+1): 439.0.

### Example 5

### ethyl 3-[4-[(4aR,8aS)-2-oxo-3,4,4a,5,6,7,8,8a-octahydroquinazolin-1-yl]-1-piperidyl]pyrrolidine-1-carboxylate (mixture of diatereoisomers)

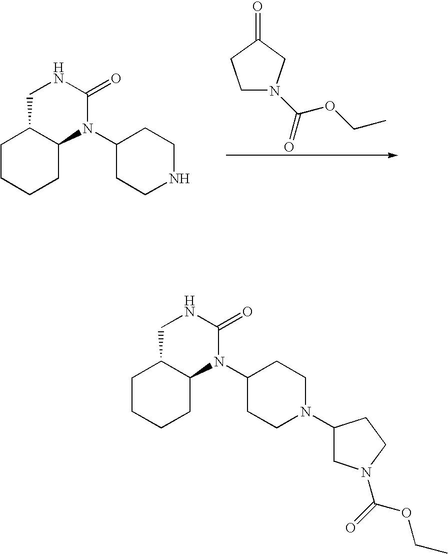

A solution of (4aR,8aS)-1-(4-piperidyl)-3,4,4a,5,6,7,8,8a-octahydroquinazolin-2-one (HCl salt, 0.1316 g, 0.48 mmol) in MeOH (5 mL) was treated with MP-carbonate resin (3.07 mmol/g, 0.63 g, 1.9 mmol) and stirred for 1 hour. The resin was filtered off, washed well with MeOH. The filtrate was concentrated in vacuo to give (4aR,8aS)-1-(4-piperidyl)-3,4,4a,5,6,7,8,8a-octahydroquinazolin-2-one as its free base form. The residue was dissolved in CH₂Cl₂ (5 mL), and ethyl 3-oxopyrrolidine-1-carboxylate (0.076 g, 0.48 mmol) and acetic acid (5.50 μL, 0.10 mmol) were added. The reaction mixture was stirred at room temperature for 45 minutes and then sodium triacetoxyborohydride (0.143 g, 0.67 mmol) was added. The reaction mixture was stirred at room temperature for 136 hours. Saturated aqueous NaHCO₃ (5 mL) was added, the mixture was loaded onto a Varian ChemElut extraction cartridge, and the product was eluted with CH₂Cl₂ (3×8 mL). The eluant was concentrated in vacuo. The crude product was purified by preparative LC/MS (high pH) to give the title compound (gradient 35-55% CH₃CN in H₂O) as a mixture of diastereomers (27.4%) as a white solid. 1H NMR (400 MHz, CHLOROFORM-D) δ ppm. 0.93-1.41 (m, 7H), 1.52-2.51 (m, 14H), 2.64-3.40 (m, 7H), 3.45-3.83 (m, 3H), 4.11 (q, J=7.3 Hz, 2H), 4.71 (d, J=3.5 Hz, 1H). Exact mass calculated for C₂₀H₃₄N₄O₃+H: 379.2704. Found: 379.2704.

### Example 6

### Propan-2-yl 4-[4-[(4aR,8aS)-3-methyl-2-oxo-4a,5,6,7,8,8a-hexahydro-4H-quinazolin-1-yl]-1-piperidyl]piperidine-1-carboxylate

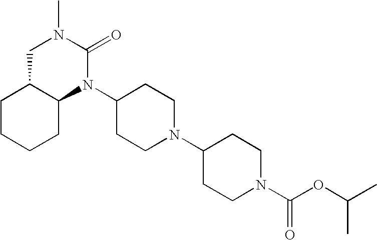

### Step A. The preparation of tert-butyl 4-[(4aR,8aS)-3-methyl-2-oxo-4a,5,6,7,8,8a-hexahydro-4H-quinazolin-1-yl]piperidine-1-carboxylate

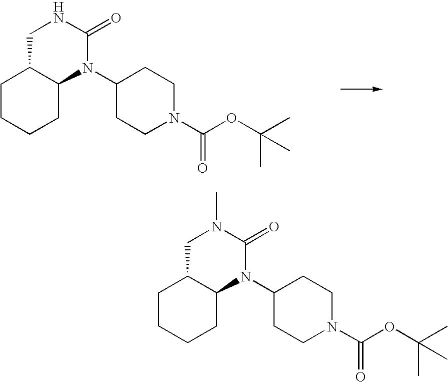

A solution of tert-butyl 4-[(4aR,8aS)-2-oxo-3,4,4a,5,6,7,8,8a-octahydroquinazolin-1-yl]piperidine-1-carboxylate (101 mg, 0.3 mmol) in dry DMF (2 mL) was added with 60% NaH (36 mg, 0.9 mmol). The reaction mixture was stirred at room temperature for 30 minutes. Methyl iodide (64 mg, 0.45 mmol) was added and the reaction mixture was stirred at room temperature for 12 hours. The solvent was removed in vacuo. The residue was taken up in dichloromethane (20 mL) and extracted with water (10 mL). The phases were separated and the aqueous phase was extracted with dichloromethane (10 mL). The combined organic phases were washed with brine, dried over Na₂SO₄ and filtered. The solvents were removed in vacuo to give the title compound, which was used for the next step without further purification. MS (M+1): 353.2.

### Step B. The preparation of (4aR,8aS)-3-methyl-1-(4-piperidyl)-4a,5,6,7,8,8a-hexahydro-4H-quinazolin-2-one

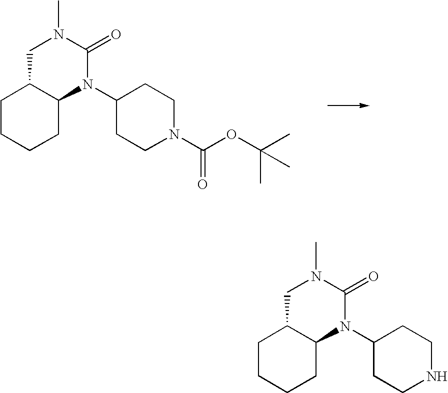

A solution of tert-butyl 4-[(4aR,8aS)-3-methyl-2-oxo-4a,5,6,7,8,8a-hexahydro-4H-quinazolin-1-yl]piperidine-1-carboxylate (0.3 mmol) in 4N HCl in dioxane (2 mL) was stirred at room temperature for 3 hours. The solvents were removed in vacuo to give the tile compound, which was used in the next step without further purification. MS (M+1): 252.2.

### Step C. The preparation of propan-2-yl 4-[4-[(4aR,8aS)-3-methyl-2-oxo-4a,5,6,7,8,8a-hexahydro-4H-quinazolin-1-yl]-1-piperidyl]piperidine-1-carboxylate

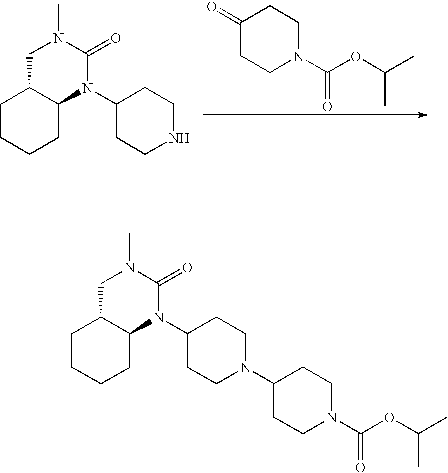

Following the analogous procedure described in step E of the example 2, the title compound was prepared from (4aR,8aS)-3-methyl-1-(4-piperidyl)-4a,5,6,7,8,8a-hexahydro-4H-quinazolin-2-one (HCl salt, 0.3 mmol) and isopropyl 4-oxopiperidine-1-carboxylate (56 mg, 0.3 mmol). The crude product was purified by preparative LC/MS (high pH) to give the title compound as white solid (29 mg, 23% over three steps). 1H NMR (400 MHz, METHANOL-D4) δ ppm 0.94-1.10 (m, 2H), 1.13 (d, J=6.25 Hz, 6H), 1.22-1.40 (m, 4H), 1.55-1.68 (m, 4H), 1.71-1.90 (m, 4H), 2.24-2.33 (m, 2H), 2.34-2.48 (m, 3H), 2.57-2.73 (m, 3H), 2.77 (s, 3H), 2.82-2.99 (m, 3H), 3.01-3.12 (m, 2H), 3.35-3.51 (m, 1H), 4.09 (d, J=13.28 Hz, 2H), 4.66-4.76 (m, 1H). MS (M+1): 421.3.

### Example 7

### Ethyl 4-[4-[(4aR,8aS)-2-oxo-3,4,4a,5,6,7,8,8a-octahydroquinazolin-1-yl]-1-piperidyl]-4-methyl-piperidine-1-carboxylate

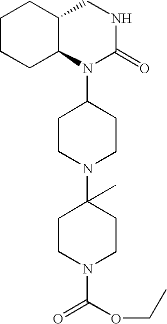

### Step A. The preparation of tert-butyl 4-[4-[[(1S,2R)-2-(azidomethyl)cyclohexyl]amino]-1-piperidyl]-4-methyl-piperidine-1-carboxylate

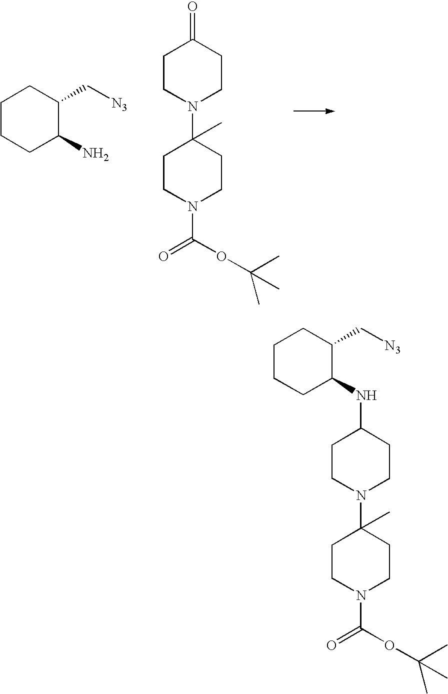

A solution of (1S,2R)-2-(azidomethyl)cyclohexan-1-amine (HCl salt, 0.510 g, 2.69 mmol) in MeOH (20 mL) was added with triethylamine (0.374 mL, 2.69 mmol) followed by tert-butyl 4-(4-oxo-1-piperidyl)piperidine-1-carboxylate (0.796 g, 2.69 mmol). The reaction mixture was stirred at room temperature for 15 minutes. A solution of zinc chloride (0.183 g, 1.34 mmol) and sodium cyanoborohydride (0.253 g, 4.03 mmol) in MeOH (2 mL) was added drop wise. The reaction mixture was stirred at room temperature overnight. The solvents were removed in vacuo. Ethylacetate was then added (100 mL) and the mixture was washed with a solution of 1 N NaOH (10 mL). The aqueous phase was extracted with ethylacetate (2×20 mL) and the combined organic phases were concentrated in vacuo. The residue was purified by flash chromatography (dichloromethane/MeOH) to provide the title compound (1 g, 86%) MS: 435.36.

### Step B. The preparation of tert-butyl 4-[4-[[(1S,2R)-2-(aminomethyl)cyclohexyl]amino]-1-piperidyl]piperidine-1-carboxylate

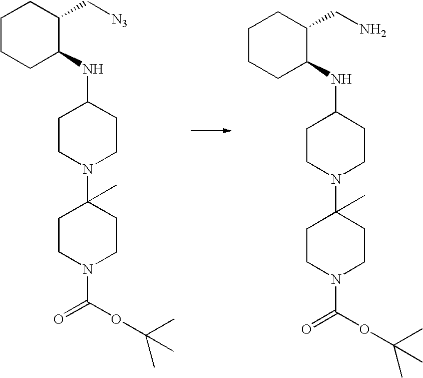

A solution of tert-butyl 4-[4-[[(1S,2 R)-2-(azidomethyl)cyclohexyl]amino]-1-piperidyl]piperidine-1-carboxylate (0.6 g, 1.38 mmol) in MeOH (10 mL) was added with platinum(IV) oxide (100 mg, 0.44 mmol). The reaction mixture stirred under hydrogen atmosphere (45 psi) for 48 hours. The catalyst was then filtered off. The filtrate was concentrated in vacuo to give the title compound (0.78 g), which was used for the next step without any further purification. MS (M+1): 409.40.

### Step C. The preparation of tert-butyl 4-[4-[(4aR,8aS)-2-oxo-3,4,4a,5,6,7,8,8a-octahydroquinazolin-1-yl]-1-piperidyl]-4-methyl-piperidine-1-carboxylate

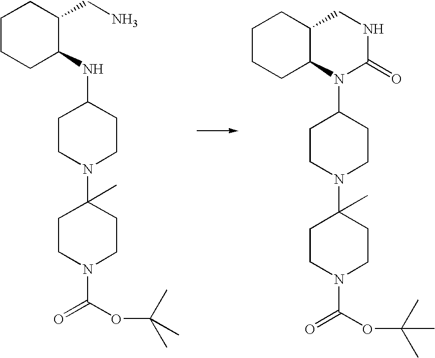

A solution of tert-butyl 4-[4-[[(1S,2R)-2-(aminomethyl)cyclohexyl]amino]-1-piperidyl]piperidine-1-carboxylate (0.78 g, 1.91 mmol) in acetonitrile (10 mL) was added with 1,1-carbonyldiimidazole (0.371 g, 2.29 mmol). The reaction mixture was stirred at room temperature for 3 hours. Concentrated in vacuo, diluted in dichloromethane (60 mL) and washed with 1N NaOH. Aqueous phase was extracted with dichloromethane and the combined organic phases were dried and concentrated in vacuo to give the title compound, which was used for the subsequent step without further purification. MS (M+1): 435.36.

### Step D: The preparation of (4aR,8aS)-1-[1-(4-methyl-4-piperidyl)-4-piperidyl]-3,4,4a,5,6,7,8,8a-octahydroquinazolin-2-one

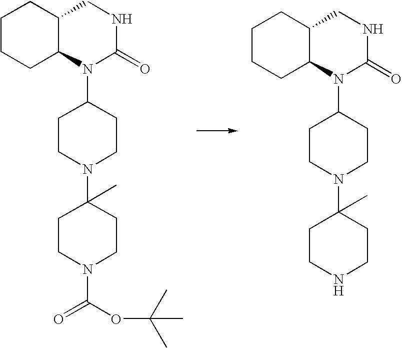

A solution of tert-butyl 4-[4-[(4aR,8aS)-2-oxo-3,4,4a,5,6,7,8,8a-octahydroquinazolin-1-yl]-1-piperidyl]-4-methyl-piperidine-1-carboxylate in MeOH (50 mL) and 4M HCl in dioxane (10 mL, 40.00 mmol) was stirred at room temperature over night. The reaction mixture was concentrated in vacuo to provide the title compound (0.4 g), which was used for the next step without any further purification. MS (M+1): 335.28.

### Step E. The preparation of ethyl 4-[4-[(4aR,8aS)-2-oxo-3,4,4a,5,6,7,8,8a-octahydroquinazolin-1-yl]-1-piperidyl]-4-methyl-piperidine-1-carboxylate

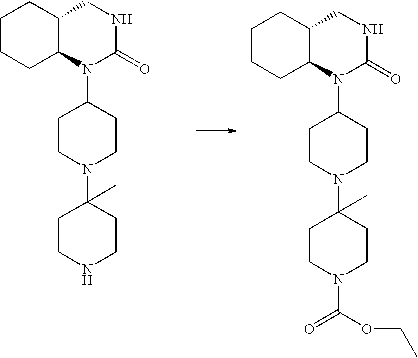

A solution of (4aR,8aS)-1-[1-(4-methyl-4-piperidyl)-4-piperidyl]-3,4,4a,5,6,7,8,8a-octahydroquinazolin-2-one (HCl salt, 0.2 g, 0.49 mmol) in dichloromethane (4 mL) was added with triethylamine (0.204 mL, 1.47 mmol) followed by ethyl chloroformate (0.056 mL, 0.59 mmol) at 0° C. The reaction mixture was stirred at room temperature for 2 hours. Diluted with dichloromethane and washed with 1N NaOH. The organic phase was separated and aqueous phase was extracted with dichloromethane. The combined organic phases were dried and concentrated in vacuo. The residue was then purified by preparative LC/MS (high pH) (40-60% MeCN in water) to provide the title compound as a white solid (38 mg). 1H NMR (400 MHz, CHLOROFORM-D) δ ppm 0.86 (s, 3H), 0.97-1.40 (m, J=7.03, 7.03 Hz, 6H), 1.22 (t, J=7.03 Hz, 3H), 1.54-1.93 (m, 8H), 1.98-2.24 (m, 4H), 2.33 (d, J=11.33 Hz, 1H), 2.72-3.04 (m, 5H), 3.22-3.41 (m, 2H), 3.41-3.59 (m, 2H), 3.55-3.74 (m, 1H), 4.09 (q, J=7.03 Hz, 2H), 4.83 (d, J=5.08 Hz, 1H). MS (M+1): 407.30.

### Example 8

### Propan-2-yl 4-[4-[(4aR,8aS)-2-oxo-3,4,4a,5,6,7,8,8a-octahydroquinazolin-1-yl]-1-piperidyl]-4-methyl-piperidine-1-carboxylate

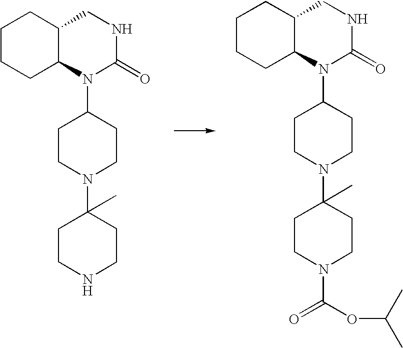

A solution of (4aR,8aS)-1-[1-(4-methyl-4-piperidyl)-4-piperidyl]-3,4,4a,5,6,7,8,8a-octahydroquinazolin-2-one (HCl salt, 0.2 g, 0.49 mmol) in dichloromethane (4 mL) was added with triethylamine (0.205 mL, 1.47 mmol). A solution of isopropyl chloroformate (0.589 mL, 0.59 mmol) in dichloromethane (1 mL) was added dropwise at 0° C. The reaction mixture was stirred at 0° C. for 2 hours and quenched with ice. The mixture was diluted in dichloromethane, 1N NaOH was then added and phases were separated. Aqueous phase was extracted with dichloromethane and the combined organic phases were dried and concentrated in vacuo. The residue was then purified by preperative LC/MS (high pH) (40-60% MeCN in water) to provide to give the title compound as a white solid (38.5 mg). 1H NMR (400 MHz, CHLOROFORM-D) δ ppm 0.85 (s, 3H), 0.94-1.12 (m, 1H), 1.10-1.37 (m, J=6.25 Hz, 5H), 1.19 (d, J=6.25 Hz, 6H), 1.47-1.88 (m, 8H), 1.97-2.39 (m, 7H), 2.77-3.01 (m, 3H), 3.25-3.51 (m, 4H), 3.50-3.69 (m, 1H), 4.68-4.91 (m, 1H), 4.90-5.01 (m, 1H). MS (M+1): 421.3. MS (M+1): 421.31.

### Example 9

### Ethyl 4-[4-[(1S,6S)-9-oxo-7-oxa-10-azabicyclo[4.4.0]dec-10-yl]-1-piperidyl]piperidine-1-carboxylate

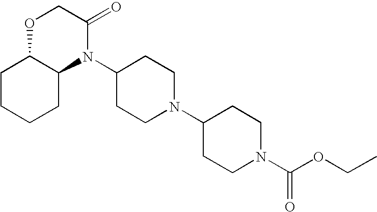

### Step A. The preparation of tert-butyl 4-[[(1S,2S)-2-phenylmethoxycyclohexyl]amino]piperidine-1-carboxylate

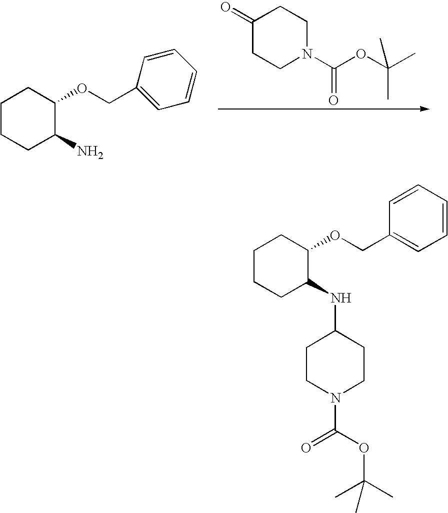

A solution of (1S,2S)-2-phenylmethoxycyclohexan-1-amine (3.75 g, 18.3 mmol) and tert-butyl 4-oxocyclohexanecarboxylate (5.44 g, 18.3 mmol) in dichloromethane (100 mL) was added with sodium triacetoxyborohydride (5.81 g, 27.5 mmol). The reaction mixture was stirred at room temperature for 12 hours. Saturated aqueous NaHCO₃ (30 mL) was added and the phases were separated. The aqueous phase was extracted with dichloromethane (2×30 mL). The combined organic phases were washed with brine, dried over Na₂SO₄ and filtered. The solvents were removed in vacuo to give the title compound (6.45 g, 91%), which was used for the next step without further purification. MS (M+1): 389.3.

### Step B. The preparation of tert-butyl 4-[[(1S,2S)-2-hydroxycyclohexyl]amino]piperidine-1-carboxylate

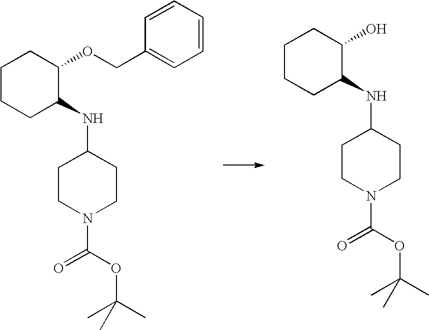

A solution of tert-butyl 4-[[(1S,2S)-2-phenylmethoxycyclohexyl]amino]piperidine-1-carboxylate (16.6 mmol) in EtOH (80 mL) was added with cyclohexene (20 mL) followed by 20% Pd(OH)₂/C (0.5 g). The reaction mixture was heated under reflux for 12 hours. The catalyst was filtered off and the filtrate was concentrated in vacuo to give the title compound as white solid (5.24 g, 98%), which was used for the next step without further purification. MS (M+1): 299.1.

### Step C. The preparation of tert-butyl 4-[(2-chloroacetyl)-[(1S,2S)-2-hydroxycyclohexyl]amino]piperidine-1-carboxylate

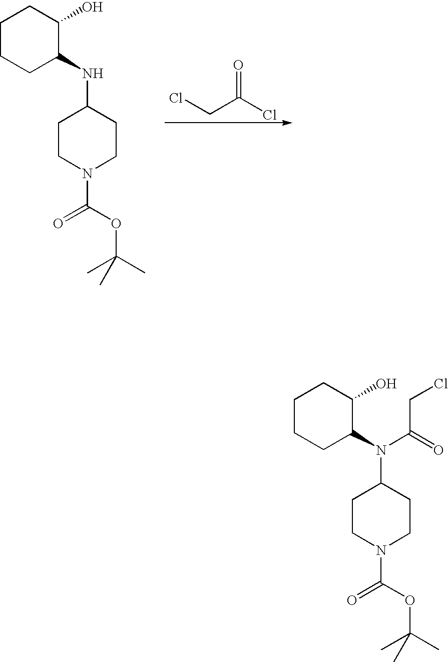

A solution of tert-butyl 4-[[(1S,2S)-2-hydroxycyclohexyl]amino]piperidine-1-carboxylate (895 mg, 3.0 mmol) in dichloromethane (30 mL) was added with chloroacetyl chloride (0.32 mL, 4.1 mmol) followed by triethyl amine (0.46 mL, 3.3 mmol). The reaction mixture was stirred at room temperature for 18 hours. Saturated aqueous NaHCO₃ (5 mL) solution was added and the phases were separated. The aqueous phase was extracted with dichloromethane (2×10 mL). The combined organic phases were washed with brine, dried over Na₂SO₄ and filtered. The solvents were removed in vacuo to give the title compound, which was used for the subsequent step without further purification (1.08 g, 96%). MS (M+1): 375.2.

### Step D. The preparation of tert-butyl 4-[(1S,6S)-9-oxo-7-oxa-10-azabicyclo[4.4.0]dec-10-yl]piperidine-1-carboxylate

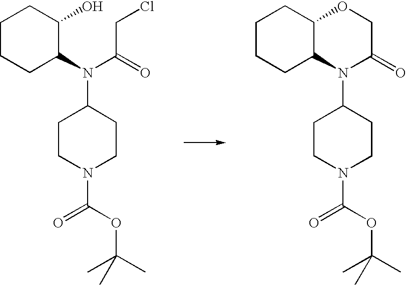

A solution of tert-butyl 4-[(2-chloroacetyl)-[(1S,2S)-2-hydroxycyclohexyl]amino]piperidine-1-carboxylate (1.08 g, 2.88 mmol) in dry THF (30 mL) at 0° C. was added with ^{t}BUOK (5.76 mmol). The reaction mixture was allowed to warm to room temperature and stirred at room temperature for 12 hours. Water (5 mL) was added and the phases were separated. The aqueous phase was extracted with dichloromethane (2×20 mL). The combined organic phases were washed with brine, dried over Na₂SO₄ and filtered. The solvents were removed in vacuo to give the title compound as white solid, which was used for the subsequent step without further purification (0.81 g, 83%). MS (M+1): 339.3.

### Step E. The preparation of (1S,6S)-5-(4-piperidyl)-2-oxa-5-azabicyclo[4.4.0]decan-4-one

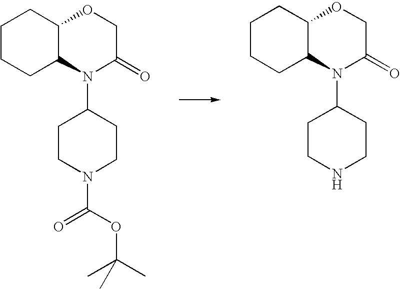

tert-butyl 4-[(1S,6S)-9-oxo-7-oxa-10-azabicyclo[4.4.0]dec-10-yl]piperidine-1-carboxylate, 0.4 mmol) was treated with 4N HCl (2 mL). The reaction mixture was stirred at room temperature for 5 hours. The solvent was removed in vacuo to give the title compound, which was used for the next step without further purification. MS (M+1): 239.2.

### Step F. The preparation of ethyl 4-[4-[(1S,6S)-9-oxo-7-oxa-10-azabicyclo[4.4.0]dec-10-yl]-1-piperidyl]piperidine-1-carboxylate

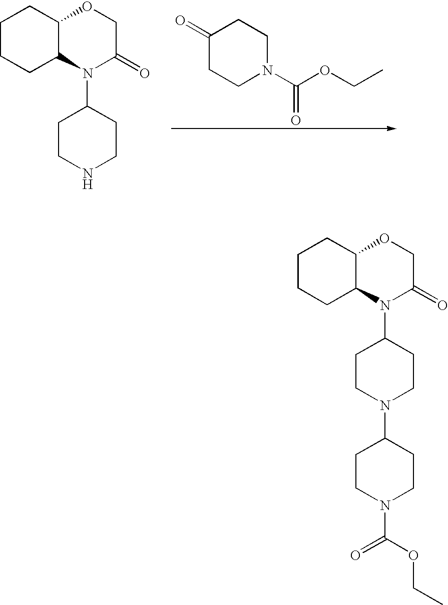

A solution of (1S,6S)-5-(4-piperidyl)-2-oxa-5-azabicyclo[4.4.0]decan-4-one (HCl salt, 0.4 mmol) and ethyl 4-oxopiperidine-1-carboxylate (69 mg, 0.4 mmol) in dichloromethane (10 mL) was added with triethyl amine (0.4 mmol) followed by sodium triacetoxyborohydride (127 mg, 0.6 mmol). The reaction mixture was stirred at room temperature for 12 hours. Saturated aqueous NaHCO₃ (5 mL) was added and the phases were separated. The aqueous phase was extracted with dichloromethane (2×20 mL). The combined organic phases were washed with brine, dried over Na₂SO₄ and filtered. The solvent was removed in vacuo. The residue was purified by preparative LC/MS to give the title compound (32 mg, 20% over3 steps). 1H NMR (400 MHz, CHLOROFORM-D) δ ppm 1.12-1.36 (m, 2H), 1.25 (t, J=7.13 Hz, 3H), 1.37-1.51 (m, 3H), 1.64-1.87 (m, 8H), 1.99-2.21 (m, 2H), 2.22-2.34 (m, 2H), 2.39-2.53 (m, 2H), 2.66-2.82 (m, 2H), 2.88-3.03 (m, 2H), 3.15-3.34 (m, 2H), 3.83-4.00 (m, 1H), 4.07-4.32 (m, 6H). MS (M+1): 394.0.

### Example 10

### Propan-2-yl 4-[4-[(1S,6S)-9-oxo-7-oxa-10-azabicyclo[4.4.0]dec-10-yl]-1-piperidyl]piperidine-1-carboxylate

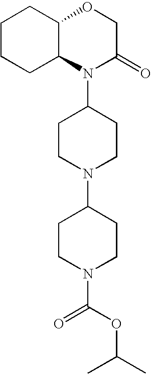

### Step A. The preparation of tert-butyl 4-[4-[(1S,6S)-9-oxo-7-oxa-10-azabicyclo[4.4.0]dec-10-yl]-1-piperidyl]piperidine-1-carboxylate

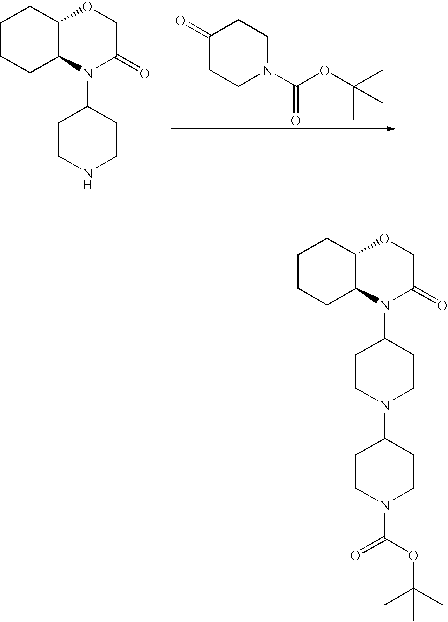

A solution of (1S,6S)-5-(4-piperidyl)-2-oxa-5-azabicyclo[4.4.0]decan-4-one (HCl salt, 2.0 mmol) and tert-butyl 4-oxopiperidine-1-carboxylate (477 mg, 2.0 mmol) in dichloromethane (30 mL) was added with triethyl amine (2.0 mmol) followed by sodium triacetoxyborohydride (635 mg, 3.0 mmol). The reaction mixture was stirred at room temperature for 12 hours. Saturated aqueous NaHCO₃ (10 mL) was added and the phases were separated. The aqueous phase was extracted with dichloromethane (2×20 mL). The combined organic phases were washed with brine, dried over Na₂SO₄ and filtered. The solvent was removed in vacuo. The residue was purified with preparative LC/MS to give the title compound (304 mg, 36% over 3 steps). 1H NMR (400 MHz, METHANOL-D4) δ ppm 1.15-1.28 (m, 1H), 1.30-1.39 (m, 8H), 1.40-1.44 (m, 9H), 1.63-1.72 (m, 2H), 1.73-1.81 (m, 2H), 1.82-1.90 (m, 2H), 1.92-2.00 (m, 1H), 2.22-2.58 (m, 6H), 2.62-2.82 (m, 1H), 3.01-3.08 (m, 2H), 3.15-3.25 (m, 1H), 3.54-3.71 (m, 1H), 4.10 (s, 2H), 4.11-4.16 (m, 1H), MS (M+1): 422.0.

### Step B. The preparation of (1S,6S)-5-[1-(4-piperidyl)-4-piperidyl]-2-oxa-5-azabicyclo[4.4.0]decan-4-one

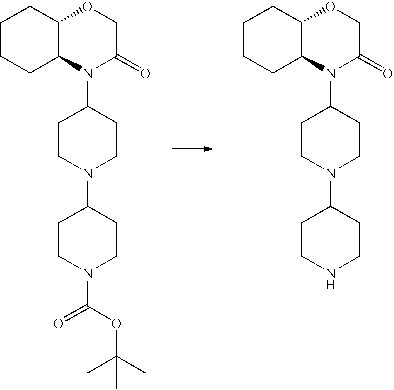

tert-butyl 4-[4-[(1S,6S)-9-oxo-7-oxa-10-azabicyclo[4.4.0]dec-10-yl]-1-piperidyl]piperidine-1-carboxylate) (304 mg, 0.72 mmol) was treated with 4N HCl (2 mL) and stirred at room temperature for 5 hours. The solvent was removed in vacuo to give the title compound (HCl salt, 213 mg, 83%), which was used for the next step without further purification. MS (M+1): 322.0.

### Step C. The preparation of propan-2-yl 4-[4-[(1S,6S)-9-oxo-7-oxa-10-azabicyclo[4.4.0]dec-10-yl]-1-piperidyl]piperidine-1-carboxylate

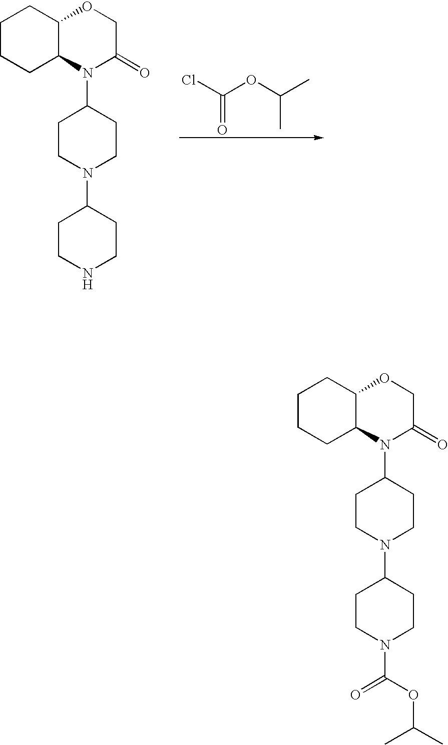

A solution of (1S,6S)-5-[1-(4-piperidyl)-4-piperidyl]-2-oxa-5-azabicyclo[4.4.0]decan-4-one (HCl salt, 71.6 mg, 0.2 mmol) in dry dichloromethane (3 mL) was added with isopropyl chloroformate (31 mg, 0.25 mmol) followed by triethyl amine (68 μL, 0.5 mmol). The reaction mixture was stirred at room temperature for 1 hour. Dichloromethane (10 mL) and saturated NaHCO₃ (5 mL) were added and the phases were separated. The aqueous phase was extracted with dichloromethane (2×10 mL). The combined organic phases were washed with brine, dried over Na₂SO₄ and filtered. The solvent was removed in vacuo. The residue was purified with preparative LC/MS to give the title compound (34 mg, 42%). 1H NMR (400 MHz, METHANOL-D4) δ ppm 1.21 (d, J=6.25 Hz, 6H), 1.28-1.46 (m, 6H), 1.62-1.70 (m, 2H), 1.74-1.89 (m, 4H), 1.91-2.02 (m, 1H), 2.19-2.34 (m, 3H), 2.34-2.44 (m, 3H), 2.46-2.56 (m, 1H), 2.62-2.83 (m, 2H), 2.94-3.08 (m, 2H), 3.14-3.24 (m, 1H), 3.52-3.70 (m, 1H), 4.10 (s, 2H), 4.11-4.20 (m, 2H), 4.77-4.89 (m, 1H). MS (M+1): 408.0.

### Example 11

### (1S,6S)-10-[1-[1-(2-methylbenzoyl)-4-piperidyl]-4-piperidyl]-7-oxa-10-azabicyclo[4.4.0]decan-9-one

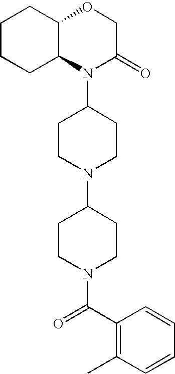

A solution of (1S,6S)-5-[1-(4-piperidyl)-4-piperidyl]-2-oxa-5-azabicyclo[4.4.0]decan-4-one (HCl salt, 71.6 mg, 0.2 mmol) in DMA (2 mL) was added with 2-methylbenzoic acid (33 mg, 0.24 mmol), HATU (0.091 g, 0.24 mmol) and followed by diisopropylethyl amine (0.042 mL, 0.24 mmol). The reaction mixture was stirred at room temperature for 3 hours and concentrated in vacuo. The residue was taken up into dichloromethane (15 mL), washed with saturated aqueous NaHCO₃ (10 mL) and brine (10 mL) and dried over Na₂SO₄. The solvent was removed in vacuo and the residue was purified by preparative LC/MS to give the title compound (53 mg, 60%). 1H NMR (400 MHz, METHANOL-D4) δ ppm 1.08-1.56 (m, 6H), 1.61-1.70 (m, 2H), 1.73-1.87 (m, 3H), 1.91-2.07 (m, 2H), 2.16-2.27 (m, 2H), 2.30 (s, 3H), 2.36-2.47 (m, 3H), 2.52-2.69 (m, 1H), 2.75-2.90 (m, 1H), 2.98-3.12 (m, 3H), 3.15-3.26 (m, 1H), 3.37-3.55 (m, 1H), 3.57-3.69 (m, 1H), 4.10 (s, 2H), 4.69-4.76 (m, 1H), 7.02-7.35 (m, 4H). MS(M+1): 440.0.

### Example 12

### (1S,6S)-10-[1-[1-(1-methylpyrrole-2-carbonyl)-4-piperidyl]-4-piperidyl]-7-oxa-10-azabicyclo[4.4.0]decan-9-one

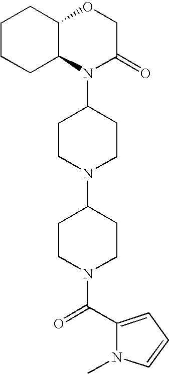

A solution of (1S,6S)-5-[1-(4-piperidyl)-4-piperidyl]-2-oxa-5-azabicyclo[4.4.0]decan-4-one (HCl salt, 71.6 mg, 0.2 mmol) in DMA (2 mL) was added with 1-methyl-1H-pyrrole-2-carboxylic acid (30 mg, 0.24 mmol), HATU (0.091 g, 0.24 mmol) and followed by diisopropylethyl amine (0.042 mL, 0.24 mmol) and stirred at room temperature for 3 hours. Concentrated in vacuo, the residue was taken up into dichloromethane (15 mL), extracted with saturated NaHCO₃ (10 mL) and brine (10 mL) and dried over Na₂SO₄. The solvent was removed in vacuo and the residue was purified by preparative LC/MS to give the title compound (48 mg, 56%). 1H NMR (400 MHz, METHANOL-D4) δ ppm 1.14-1.45 (m, 4H), 1.48-1.64 (m, 2H), 1.70-1.85 (m, 5H), 1.89-1.98 (m, 1H), 2.29-2.42 (m, 1H), 2.50-2.77 (m, 6H), 2.93-3.08 (m, 3H), 3.13-3.25 (m, 1H), 3.25-3.37 (m, 7H), 3.86 (s, 1H, rotamer), 4.10 (s, 2H, rotamer), 4.42-4.59 (m, 2H), 5.88-6.16 (m, 0.8 H, rotamer), 6.35 (dd, J=3.91, 1.56 Hz, 0.7 H, rotamer), 6.67-6.86 (m, 0.9 H, rotamer), 7.27 (dd, J=8.59, 4.30 Hz, 0.2 H, rotamer), 8.13 (d, J=7.03 Hz, 0.2 H, rotamer), 8.47 (d, J=3.12 Hz, 0.2 H, rotamer). MS (M+1): 429.0.

### Example 13

### Ethyl (3S)-3-[4-[(1S,6S)-9-oxo-7-oxa-10-azabicyclo[4.4.0]dec-10-yl]-1-piperidyl]pyrrolidine-1-carboxylate

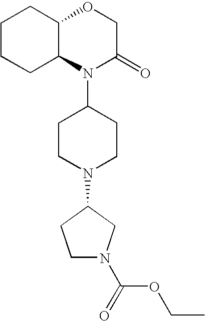

### Step A. The preparation of tert-butyl (3S)-3-[4-[[(1S,2S)-2-phenylmethoxycyclohexyl]amino]-1-piperidyl]pyrrolidine-1-carboxylate

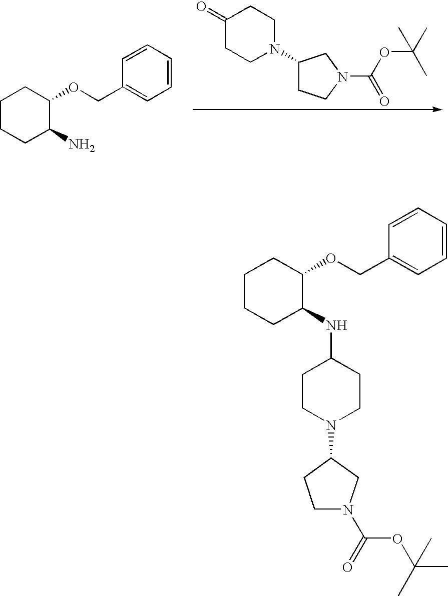

A solution of (1S,2S)-2-phenylmethoxycyclohexan-1-amine (0.287 g, 1.4 mmol) and tert-butyl (3S)-3-(4-oxo-1-piperidyl)pyrrolidine-1-carboxylate (0.376 g, 1.4 mmol) (prepared according to the method described in WO 2007142585 A1) in dichloromethane (11 mL) was added with sodium triacetoxyborohydride (0.445 g, 2.1 mmol). The reaction mixture was stirred at room temperature for 19 hours. Saturated aqueous NaHCO₃ (6 mL) was added and the phases were separated. The aqueous phase was extracted with dichloromethane (3×10 mL). The combined organic phases were dried over Na₂SO₄ and filtered. The solvent was removed in vacuo. The residue was purified by flash chromatography (4:1 CH₂Cl₂/NH₃:MeOH) to provide the title compound (0.526 g, 82%). MS (M+1): 458.3.

### Step B. The preparation of tert-butyl (3S)-3-[4-[[(1S,2S)-2-hydroxycyclohexyl]amino]-1-piperidyl]pyrrolidine-1-carboxylate

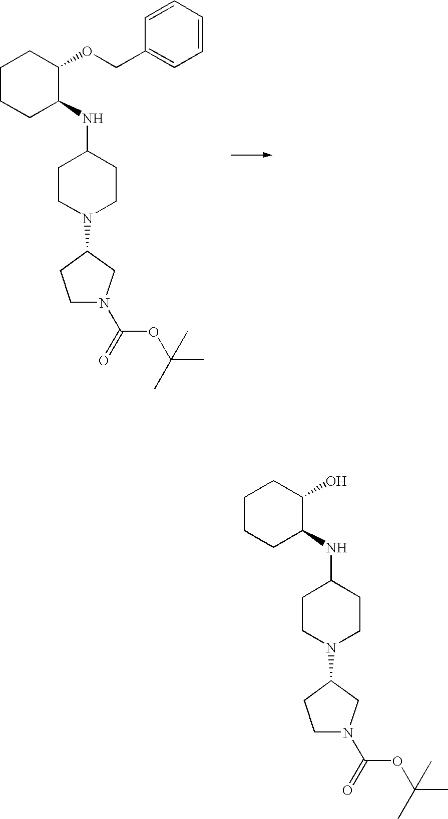

A solution of tert-butyl (3S)-3-[4-[[(1S,²S)-²-phenylmethoxycyclohexyl]amino]-1-piperidyl]pyrrolidine-1-carboxylate (from step A) (1.1 mmol) in MeOH (15 mL) was added with ammonium formate (0.345 g, 5.5 mmol) and Pd(OH)₂ (20 wt. % on carbon, 0.4 g). The reaction mixture was heated at reflux for 5 hours. The reaction was cooled and filtered through a pad of Celite. The Celite was washed well with additional MeOH, and the filtrate was concentrated in vacuo to give the title compound (0.380 g, 90%), which was used in the next step without further purification. MS (M+1): 368.2.

### Step C. The preparation of tert-butyl (3S)-3-[4-[(1S,6S)-9-oxo-7-oxa-10-azabicyclo[4.4.0]dec-10-yl]-1-piperidyl]pyrrolidine-1-carboxylate

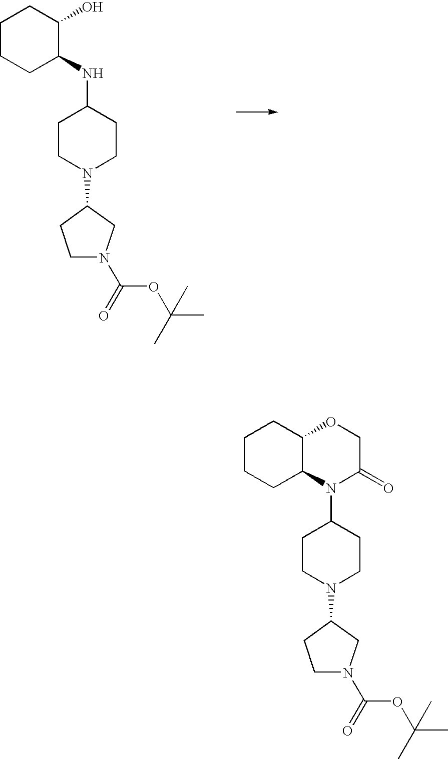

A solution of tert-butyl (3S)-3-[4-[[(1S,2S)-2-hydroxycyclohexyl]amino]-1-piperidyl]pyrrolidine-1-carboxylate (0.235 g, 0.639 mmol) in dry dichloromethane (6 mL) was added with chloroacetyl chloride (0.071 mL, 0.89 mmol) and triethylamine (0.098 mL, 0.7 mmol). The reaction mixture was stirred at room temperature for 16 hours. A saturated solution of aqueous NaHCO₃ (3 mL) was added and the phases were separated. The aqueous phase was extracted with additional dichloromethane (3×5 mL). The combined organic phases were dried over Na₂SO₄, filtered, and concentrated in vacuo. The residue was dissolved in dry THF (6 mL), cooled in an ice bath, and then potassium tert-butoxide (0.136 g, 1.21 mmol) was added. The mixture was warmed to room temperature and stirred for 17 hours. Water (3 mL), brine (5 mL), and CH₂Cl₂ (10 mL) were added to the reaction, and the phases were separated. The aqueous phase was extracted with additional CH₂Cl₂ (3×8 mL), and the combined organic phases were dried over Na₂SO₄, filtered, and concentrated in vacuo. The crude product was purified by flash chromatography (9:1 CH₂Cl₂:MeOH) to provide the title compound (0.182 g, 70% over 2 steps) as a pale yellow oil that solidified on standing. 1H NMR (400 MHz, CHLOROFORM-D) δ ppm 1.06-1.42 (m, 5H), 1.44 (s, 9H), 1.52-1.86 (m, 5H), 1.95-2.32 (m, 5H), 2.35-2.47 (m, 1H), 2.67-2.85 (m, 1H), 2.91 (d, J=10.5 Hz, 1H), 2.96-3.12 (m, 2H), 3.12-3.31 (m, 3H), 3.41-3.71 (m, 2H), 3.84-4.00 (m, 1H), 4.08-4.20 (m, 1H), 4.20-4.28 (m, 1H). MS (M+1): 408.5.

### Step D. The preparation of ethyl (3S)-3-[4-[(1S,6S)-9-oxo-7-oxa-10-azabicyclo[4.4.0]dec-10-yl]-1-piperidyl]pyrrolidine-1-carboxylate

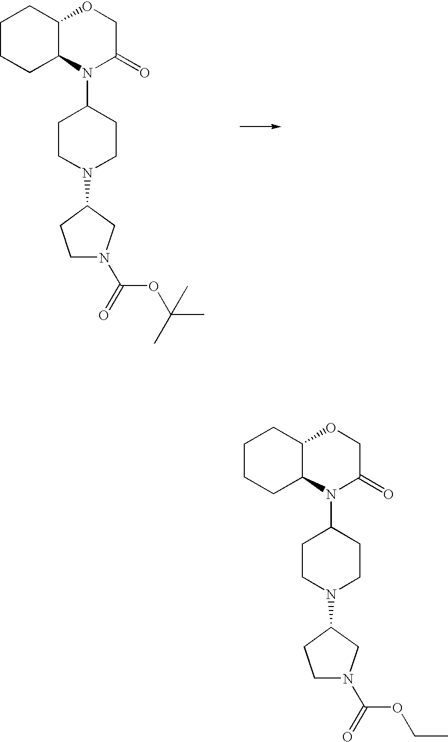

tert-butyl (3S)-3-[4-[(1S,6S)-9-oxo-7-oxa-10-azabicyclo[4.4.0]dec-10-yl]-1-piperidyl]pyrrolidine-1-carboxylate (0.1388 g, 0.34 mmol) was suspended in dioxane (1.7 mL) and water (0.68 mL) and was treated with hydrogen chloride (4 M in dioxane) (1.7 mL, 6.8 mmol). The reaction mixture was stirred at room temperature for 3 hours. Volatiles were removed in vacuo and the remaining aqueous solution was lyophilized. The resulting solid was suspended in dry dichloromethane (7 mL) and triethylamine (0.18 mL, 1.3 mmol) was added. The mixture was cooled in an ice bath, and then a solution of ethyl chloroformate (0.043 mL, 0.45 mmol) in dry dichloromethane (1 mL) was added drop wise. The reaction was stirred at 0° C. for 1.5 hours, and was then quenched with water (7 mL). The phases were separated, and the aqueous phase was extracted with additional dichloromethane (3×7 mL). The combined organic phases were dried over Na₂SO₄ and filtered. The solvent was removed in vacuo. The residue was purified by preparative LC/MS (high pH) (gradient 35-55% CH₃CN in H₂O) to provide the tile compound (0.054 g, 41% over 2 steps) as a white solid. 1H NMR (400 MHz, CHLOROFORM-D) δ ppm 1.10-1.20 (m, 1H), 1.24 (t, J=7.2 Hz, 3H), 1.27-1.50 (m, 3H), 1.58-1.88 (m, 5H), 1.95-2.34 (m, 6H), 2.40 (d, J=12.1 Hz, 1H), 2.66-3.38 (m, 7H), 3.46-3.77 (m, 2H), 3.82-4.00 (m, 1H), 4.05-4.19 (m, 3H), 4.19-4.30 (m, 1H). MS (M+1): 380.2.

### Example 14

### Propan-2-yl 4-[4-[(1R,6R)-9-oxo-7-oxa-10-azabicyclo[4.4.0]dec-10yl]-1-piperidyl]piperidine-1-carboxylate

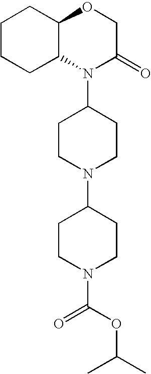

### Step A. The preparation of tert-butyl 4-[[(1R,2R)-2-phenylmethoxycyclohexyl]amino]piperidine-1-carboxylate

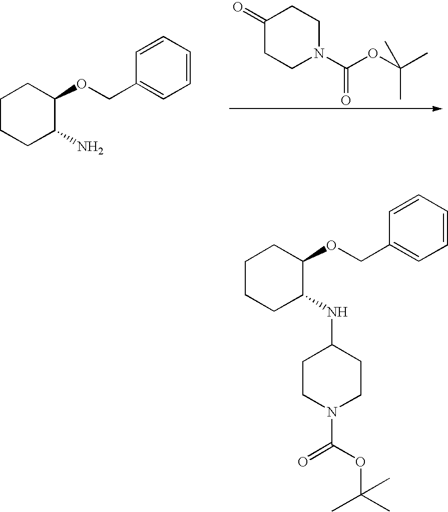

A solution of (1R,2R)-2-phenylmethoxycyclohexan-1-amine (821 mg, 4.0 mmol) and tert-butyl 4-oxopiperidine-1-carboxylate (1.19 g, 4.0 mmol) in dichloromethane (30 mL) was added with sodium triacetoxyborohydride (1.27 g, 6.0 mmol) and stirred at room temperature for 12 hours. Saturated aqueous NaHCO₃ (10 mL) was added and the phases were separated. The aqueous phase was extracted with dichloromethane (2×30 mL). The combined organic phases were washed with brine, dried over Na₂SO₄ and filtered. The solvent was removed in vacuo to provide the title compound, which was used for the next step without further purification. MS (M+1): 389.3.

### Step B. The preparation of tert-butyl 4-[[(1R,2R)-2-hydroxycyclohexyl]amino]piperidine-1-carboxylate

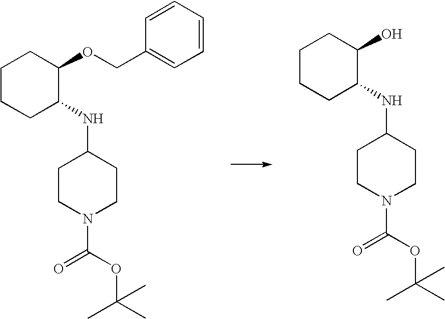

A solution of tert-butyl 4-[[(1R,2R)-2-phenylmethoxycyclohexyl]amino]piperidine-1-carboxylate (4.0 mmol) in EtOH (20 mL) and cyclohexene (10 mL) was added with 20% Pd(OH)₂/C (0.2 g). The reaction mixture was heated under reflux for 12 hours. The catalyst was filtered off and the filtrate was concentrated in vacuo to give the title compound as as a white solid (989 mg, 83% over 2 steps), which was used for the next step without further purification. MS (M+1): 299.1.

### Step C. The preparation of tert-butyl 4-[(1R,6R)-9-oxo-7-oxa-10-azabicyclo[4.4.0]dec-10-yl]piperidine-1-carboxylate

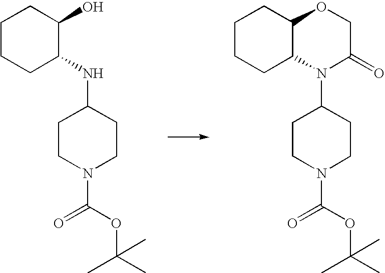

Following the analogous procedure described in step C of the example 13, the title compound was prepared from tert-butyl 4-[[(1R,2R)-2-hydroxycyclohexyl]amino]piperidine-1-carboxylate (0.419 g, 1.41 mmol). The crude product was purified by flash chromatography (9:1 CH₂Cl₂:MeOH) to give the title compound (0.204 g, 43% over two steps). 1H NMR (400 MHz, CHLOROFORM-D) δ ppm 1.11-1.42 (m, 4H), 1.45 (s, 9H), 1.59-1.71 (m, 2H), 1.74-1.87 (m, 2H), 1.96-2.33 (m, 4H), 2.70 (d, J=9.8 Hz, 2H), 3.14-3.31 (m, 2H), 3.91 (tt, J=12.3, 3.9 Hz, 1H), 4.08-4.31 (m, 4H). MS (M+1): 339.2.

### Step D. The preparation of propan-2-yl 4-[4-[(1R,6R)-9-oxo-7-oxa-10-azabicyclo[4.4.0]dec10-yl]-1-piperidyl]piperidine-1-carboxylate

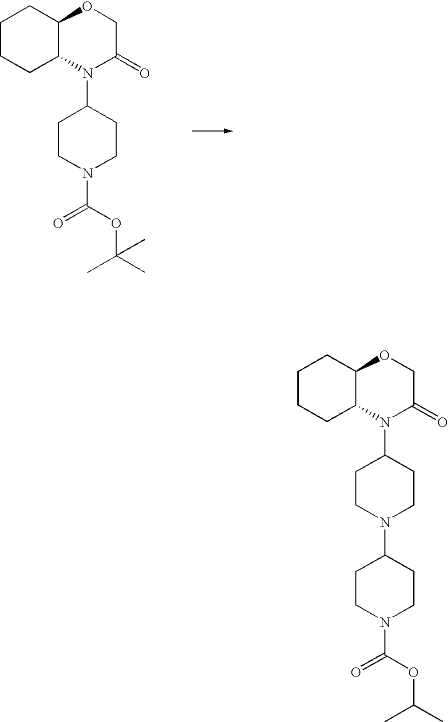

tert-butyl 4-[(1R,6R)-9-oxo-7-oxa-10-azabicyclo[4.4.0]dec-10-yl]piperidine-1-carboxylate (0.172 g, 0.51 mmol) was suspended in dioxane (2.5 mL) and water (1 mL) and the mixture was treated with hydrogen chloride (4 M in dioxane, 2.5 mL, 10 mmol). The reaction mixture was stirred at room temperature for 3 hours. The solvent was removed in vacuo and the residue lyophilized from water. The resulting solid was mixed with triethylamine (0.083 mL, 0.60 mmol) and isopropyl 4-oxopiperidine-1-carboxylate (0.100 g, 0.54 mmol) in dichloromethane (14 mL), and the resulting mixture was stirred for 30 minutes. Sodium triacetoxyborohydride (0.172 g, 0.81 mmol) was added, and the reaction mixture was stirred at room temperature for 16 hours. Saturated NaHCO₃ (7 mL) was added and the phases were separated. The aqueous phase was extracted with additional dichloromethane (3×20 mL), and the combined organic phases were dried over Na₂SO₄ and filtered. The solvent was removed in vacuo. The residue was purified by preparative LC/MS (high pH) (45-65% CH₃CN in H₂O) to provide the title compound (0.076 g, 37% over two steps) as a white solid. 1H NMR (400 MHz, CHLOROFORM-D) δ ppm 1.10-1.20 (m, 1H), 1.22 (d, J=6.2 Hz, 6H), 1.25-1.59 (m, 5H), 1.68-2.08 (m, 8H), 2.17-2.65 (m, 4H), 2.72 (t, J=11.9 Hz, 3H), 3.02-3.33 (m, 4H), 4.09-4.38 (m, J=16.4, 16.4, 16.4 Hz, 5H), 4.81-4.95 (m, 1H). MS (M+1): 408.3.

### Example 15

### Ethyl 4-[4-[(1S,6S)-9-oxo-8-oxa-10-azabicyclo[4.4.0]dec-10-yl]-1-piperidyl]piperidine-1-carboxylate

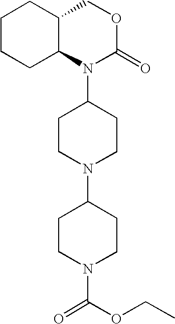

### Step A. The preparation of [(1S,2S)-2-aminocyclohexyl]methanol

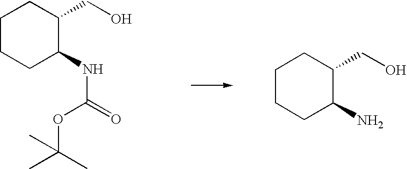

A solution of tert-butyl N-[(1S,2S)-2-(hydroxymethyl)cyclohexyl]carbamate (1.5 g, 5.02 mmol) in dioxane (20 mL) was added with a solution of 4 M HCl in dioxane (6 mL). The reaction mixture was stirred at room temperature overnight. Solvent was removed in vacuo to give the title compound (HCl salt, 1.1 g), which was used for the next step without further purification.

### Step B. The preparation of tert-butyl 4-[[(1S,2S)-2-(hydroxymethyl)cyclohexyl]amino]piperidine-1-carboxylate

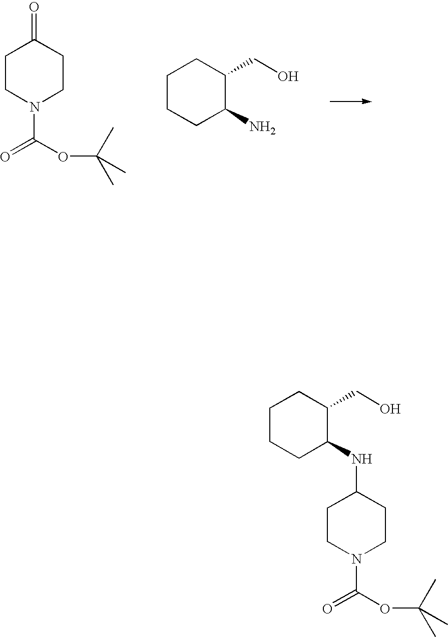

A solution of [(1S,2S)-2-aminocyclohexyl]methanol (HCl salt, 0.85 g, 5.02 mmol) in MeOH (10 mL) was added with MeONa (5.02 mmol) followed by tert-butyl 4-oxopiperidine-1-carboxylate (1.1 g, 5.53 mmol). The reaction mixture was stirred for 15 minutes at room temperature. A solution of ZnCl₂ (0.37 g, 2.72 mmol) and NaBH₃CN (0.56 g, 8.11 mmol) in MeOH (1 mL) was added dropwise and the mixture was stirred at room temperature overnight. The reaction was quenched with ice and concentrated in vacuo. The mixture was then diluted in dichloromethane and washed with 1N NaOH. The phases were separated and aqueous phase was extracted with dichloromethane. The combined organic phases were dried and concentrated in vacuo to provide the title compound, which was used for the next step without any further purification (1.88 g). MS (M+1): 313.27.

### Step C. The preparation of tert-butyl 4-[(1S,6S)-9-oxo-8-oxa-10-azabicyclo[4.4.0]dec-10-yl]piperidine-1-carboxylate

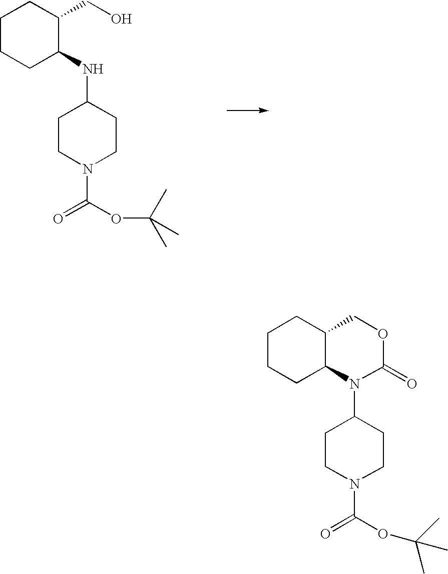

A solution of tert-butyl 4-[[(1S,2S)-2-(hydroxymethyl)cyclohexyl]amino]piperidine-1-carboxylate (1.88 g) in THF (35 mL) was added with diisopropyl ethylamine (2.84 mL, 16.33 mmol) followed by the addition of triphosgene (0.56 g, 1.89 mmol) at 0° C. The reaction mixture was stirred for 1 hour at 0° C. The solvent was removed in vacuo. The residue was dissolved in dichloromethane, 1N NaOH was added and phases were separated. Aqueous phase was extracted with dichloromethane. The combined organic phases were dried and concentrated in vacuo. The residue was purified by flash chromatography (dichloromethane/MeOH gradient) to provide the title compound (1.1 g). MS (M+1): 339.24.

### Step D. The preparation of (1S,6S)-2-(4-piperidyl)-4-oxa-2-azabicyclo[4.4.0]decan-3-one

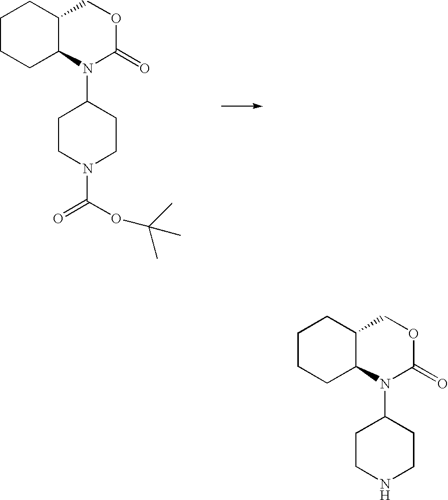

A solution of tert-butyl 4-[(1S,6S)-9-oxo-8-oxa-10-azabicyclo[4.4.0]dec-10-yl]piperidine-1-carboxylate (1.1 g, 3.25 mmol) in dioxane/MeOH (1:1, 60 mL) was added with a solution of 4M HCl in dioxane (20 mL). The reaction mixture was stirred at room temperature overnight. The solvent was removed in vacuo and the residue was purified by preparative LCMS (high pH) (10-30% MeCN in water) to provide the title compound as yellow oil (0.6 g). MS (M+1): 239.24.

### Step E. The preparation of ethyl 4-[4-[(1S,6S)-9-oxo-8-oxa-10-azabicyclo[4.4.0]dec-10-yl]-1-piperidyl]piperidine-1-carboxylate

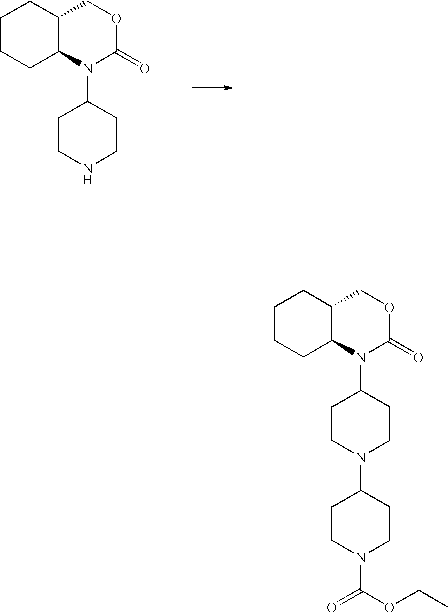

A solution of (1S,6S)-2-(4-piperidyl)-4-oxa-2-azabicyclo[4.4.0]decan-3-one (0.1 g, 0.36 mmol) in MeOH (3 mL) was added with ethyl 4-oxopiperidine-1-carboxylate (66 uL, 0.44 mmol). The reaction mixture was stirred at room temperature for 15 minutes. A solution of ZnCL₂ (25 mg, 0.18 mmol) and NaBH₃CN (38 mg, 0.55 mmol) in MeOH (1 mL) was added dropwise and the mixture was stirred at room temperature overnight. The reaction was quenched with ice and concentrated in vacuo. The residue was dissolved in dichloromethane and washed with 1N NaOH. The phases were separated and aqueous phase was extracted with dichloromethane. The combined organic phases were dried and concentrated in vacuo. The residue was then purified by preperative LC/MS (high pH) (40-60% MeCN in water) (HCl salt, 48 mg, 31%). 1H NMR (400 MHz, CHLOROFORM-D) δ ppm 0.93-1.10 (m, 1H), 1.21 (t, J=7.16 Hz, 3H), 1.11-1.25 (m, 1H), 1.25-1.45 (m, 4H), 1.60 (d, J=12.89 Hz, 1H), 1.64-1.80 (m, 6H), 1.83 (d, J=9.37 Hz, 1H), 2.11-2.26 (m, 3H), 2.24-2.35 (m, 2H), 2.40 (t, J=11.33 Hz, 1H), 2.69 (t, J=12.30 Hz, 2H), 2.83-2.98 (m, 3H), 3.32-3.49 (m, 1H), 3.76 (t, J=10.74 Hz, 1H), 3.94 (dd, J=9.57, 2.54 Hz, 1H), 4.00-4.24 (m, 2H), 4.07 (q, J=7.16 Hz, 2H). MS (M+1): 394.3.

### Example 16

### Propan-2-yl 4-[4-[(1S,6S)-9-oxo-8-oxa-10-azabicyclo[4.4.0]dec-10-yl]-1-piperidyl]piperidine-1-carboxylate

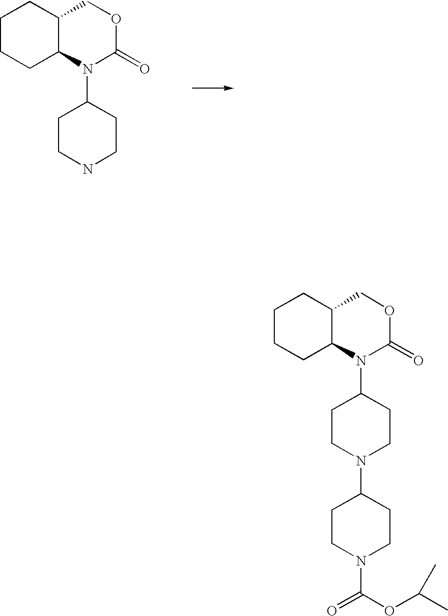

A solution of (1S,6S)-2-(4-piperidyl)-4-oxa-2-azabicyclo[4.4.0]decan-3-one (0.1 g, 0.36 mmol) in MeOH (3 mL) was added with isopropyl 4-oxopiperidine-1-carboxylate (0.08 g, 0.43 mmol). The reaction mixture was stirred at room temperature for 15 minutes. A solution of ZnCL₂ (0.3 g, 2.20 mmol) and NaBH₃CN (0.5 g, 7.24 mmol) in MeOH (1 mL) was added dropwise and the mixture was stirred at room temperature overnight. The reaction was quenched with ice and concentrated in vacuo. The residue was dissolved in dichloromethane and washed with 1N NaOH. The phases were separated and aqueous phase was extracted with dichloromethane. The combined organic phases were dried and concentrated in vacuo. The residue was then purified by preperative LC/MS (high pH ) (40-60% MeCN in water) (HCl salt, 58 mg, 34%). 1H NMR (400 MHz, CHLOROFORM-D) δ ppm 0.92-1.10 (m, 1H), 1.20 (d, J=6.25 Hz, 6H), 1.11-1.47 (m, 5H), 1.62 (d, J=10.55 Hz, 1H), 1.66-1.79 (m, 6H), 1.84 (d, J=8.20 Hz, 1H), 2.12-2.36 (m, 5H), 2.37-2.49 (m, 1H), 2.67 (t, J=12.11 Hz, 2H), 2.82-3.02 (m, 3H), 3.37-3.54 (m, 1H), 3.78 (t, J=10.74 Hz, 1H), 3.95 (dd, J=10.35, 3.32 Hz, 1H), 4.05-4.28 (m, 2H), 4.73-4.94 (m, 1H). MS (M+1): 408.29.

### Example 17

### (±)(trans)-10-[1-[1-(3-methoxythiophene-2-carbonyl)-4-piperidyl]-4-piperidyl]-8-oxa-10-azabicyclo[4.4.0]decan-9-one

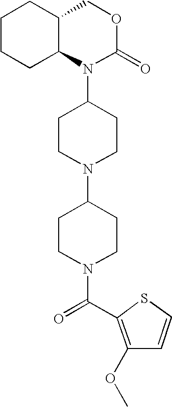

### Step A. The preparation of tert-butyl 4-[[(trans)-2-(hydroxymethyl)cyclohexyl]amino]piperidine-1-carboxylate

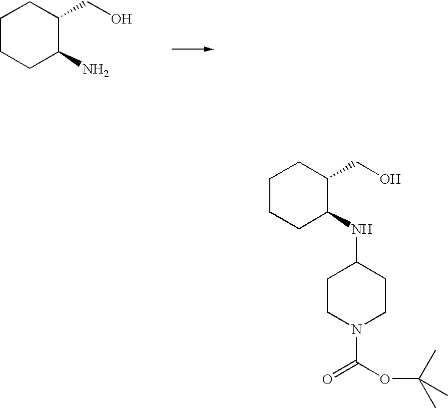

Following the analogous procedure described in step B of the Example 15, the title compound was prepared from [(trans)-2-aminocyclohexyl]methanol (HCl salt, 3.87 mmol) and tert-butyl 4-oxopiperidine-1-carboxylate (3.87 mmol). The crude product (1.2 g) was used for the next step without any further purification. MS (M+1): 313.32.

### Step B. The preparation of tert-butyl 4-[(trans)-9-oxo-8-oxa-10-azabicyclo[4.4.0]dec-10-yl]piperidine-1-carboxylate

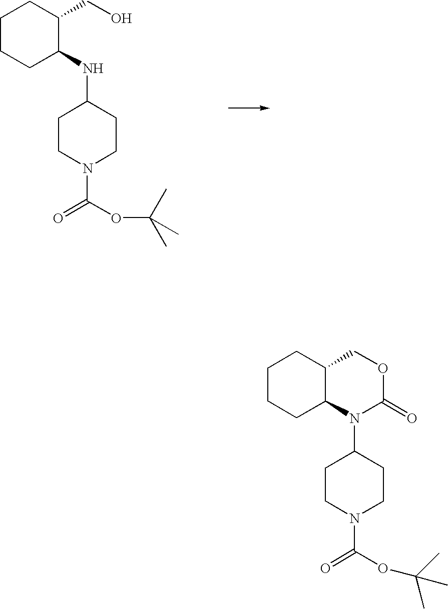

Following the analogous procedure described in step C of the Example 15, the title compound was prepared from tert-butyl 4-[[(trans)-2-(hydroxymethyl)cyclohexyl]amino]piperidine-1-carboxylate (3.20 mmol). The crude product was used for the next step without any further purification. MS (M+1): 339.24.

### Step C. The preparation of (trans)-2-(4-piperidyl)-4-oxa-2-azabicyclo[4.4.0]decan-3-one

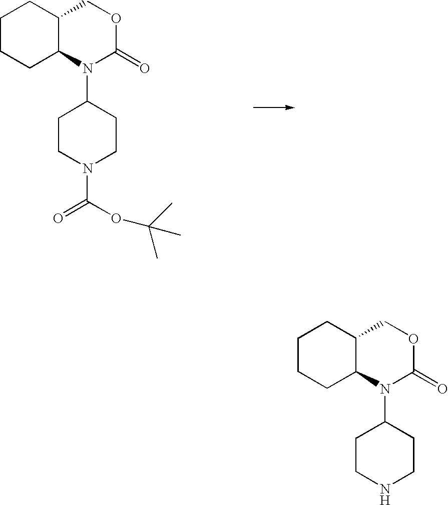

Following the analogous procedure described in step D of the Example 15, the title compound was prepared from tert-butyl 4-[(trans)-9-oxo-8-oxa-10-azabicyclo[4.4.0]dec-10-yl]piperidine-1-carboxylate (3.20 mmol). The crude product was purified by preperative LC/MS (high pH) (15-35% MeCN in water) to provide the title compound as a yellow oil (0.53 g). MS (M+1): 239.06.

### Step D. The preparation of tert-butyl 4-[4-[(trans)-9-oxo-8-oxa-10-azabicyclo[4.4.0]dec-10-yl]-1-piperidyl]piperidine-1-carboxylate

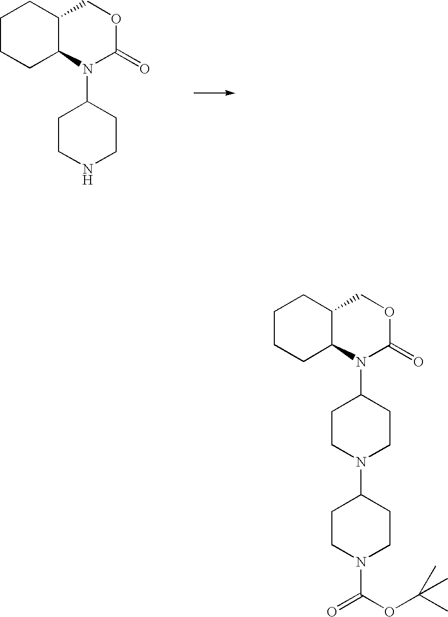

Following the analogous procedure described in step E of the Example 15, the title compound was prepared from (trans)-2-(4-piperidyl)-4-oxa-2-azabicyclo[4.4.0]decan-3-one (0.75 mmol). The crude product was purified by preperative LC/MS (high pH ) (35-55% MeCN in Water) to provide the title compound as a white solid (90 mg, 32%). MS (M+1): 422.43.

### Step E. The preparation of (trans)-2-[1-(4-piperidyl)-4-piperidyl]-4-oxa-2-azabicyclo[4.4.0]decan-3-one

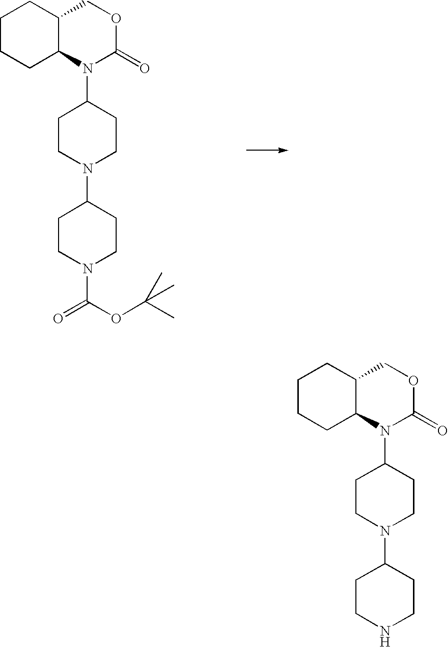

A solution of tert-butyl 4-[4-[(trans)-9-oxo-8-oxa-10-azabicyclo[4.4.0]dec-10-yl]-1-piperidyl]piperidine-1-carboxylate (0.11 mmol) in dioxane (2 mL) was added with 4M HCl in dioxane (1 mL). The reaction mixture was stirred at room temperature overnight. The solvents were removed in vacuo to give the title compound, which was used for the next step without any further purification. MS (M+1): 322.27.

### Step F. The preparation of (trans)-10-[1-[1-(3-methoxythiophene-2-carbonyl)-4-piperidyl]-4-piperidyl]-8-oxa-10-azabicyclo[4.4.0]decan-9-one

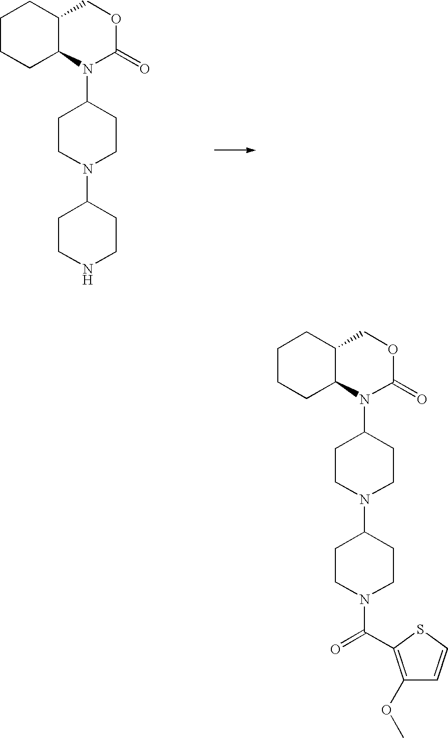

A solution of (trans)-2-[1-(4-piperidyl)-4-piperidyl]-4-oxa-2-azabicyclo[4.4.0]decan-3-one (0.1 mmol) in DMF (3 mL) was added with diisopropylethyl amine (0.3 mmol) and 3-methoxythiophene-2-carboxylic acid (0.1 mmol). HATU (0.1 mmol) was then added and the mixture was stirred at room temperature overnight. Concentrated in vacuo and the residue was diluted in dichloromethane. 1N NaOH was then added and phases were separated. Aqueous phase was then extracted with dichloromethane; the combined organic phases were dried and concentrated in vacuo. The crude product was then purified by preparative LC/MS (high pH) (30-50% MeCN in water) to provide the title compound as a white solid (16 mg). 1H NMR (400 MHz, CHLOROFORM-D) δ ppm 0.96-1.13 (m, 1H), 1.14-1.40 (m, 3H), 1.41-1.57 (m, 2H), 1.64 (d, J=11.33 Hz, 1H), 1.68-1.92 (m, 6H), 1.96-2.16 (m, 3H), 2.16-2.31 (m, 4H), 2.35 (d, J=12.11 Hz, 1H), 2.52 (t, J=11.33 Hz, 1H), 2.77-3.01 (m, 4H), 3.43-3.54 (m, 1H), 3.79 (t, J=10.94 Hz, 1H), 3.86 (s, 3H), 3.97 (dd, J=10.55, 3.12 Hz, 1H), 4.09-4.48 (m, 1H), 6.75 (d, J=5.47 Hz, 1H), 7.18-7.41 (m, 1H). MS (M+1): 462.3.

### Example 18 (Isomer 1) and Example 19 (Isomer 2)

### Ethyl 3-methyl-3-(4-((4aS,8aS)-3-oxo-2H-benzo[b][1,4]oxazin-4(3H,4aH,5H,6H,7H,8H,8aH)-yl)piperidin-1-yl)pyrrolidine-1-carboxylate (Isomer 1 and Isomer 2)

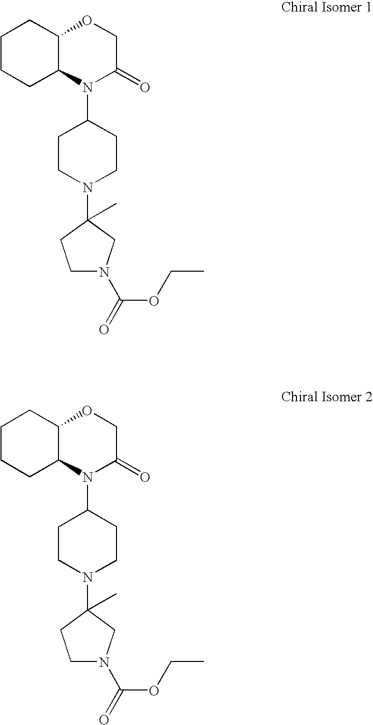

### Step A: The preparation of tert-butyl 3-(4-((1S,2S)-2-hydroxycyclohexylamino)piperidin-1-yl)-3-methylpyrrolidine-1-carboxylate

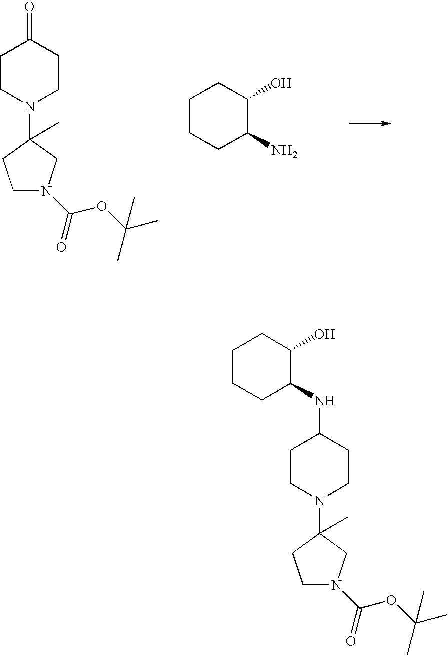

A solution of (1S,2S)-2-aminocyclohexanol (0.300 g, 2.60 mmol), tert-butyl 3-methyl-3-(4-oxopiperidin-1-yl)pyrrolidine-1-carboxylate (0.736 g, 2.60 mmol) and acetic acid (0.149 ml, 2.60 mmol) in CH2Cl2 (26.0 ml) was stirred at room temperature for 30 min. Sodium triacetoxyhydroborate (0.552 g, 2.60 mmol) was added, and the reaction mixture was stirred at room temperature for 10 h. A 1N NaOH solution (50 mL) was added, and phases were separated. The aqueous phase was extracted with CH2Cl2 (3×50 ml). The combined organic phases were washed with brine (1×50 mL), and the dried over sodium sulfate. The solvent was concentrated under reduced pressure to afford crude tert-butyl 3-(4-((1S,2S)-2-hydroxycyclohexylamino)piperidin-1-yl)-3-methylpyrrolidine-1-carboxylate (0.994 g) as a solid. The crude was used in the next step without any further purification. MS: 326.16 (M+1-56).

### Step B: The preparation of tert-butyl 3-methyl-3-(4-((4aS,8aS)-3-oxo-2H-benzo[b][1,4]oxazin-4(3H,4aH,5H,6H,7H,8H,8aH)-yl)piperidin-1-yl)pyrrolidine-1-carboxylate

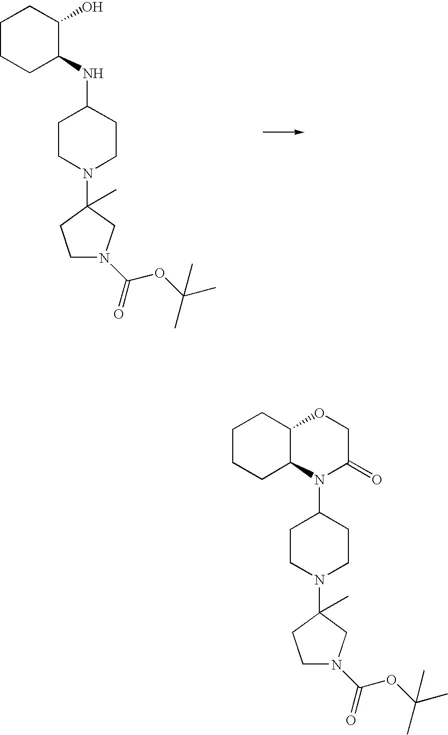

Following the similar procedure described in Example 13, Step C: tert-butyl 3-methyl-3-(4-((4aS,8aS)-3-oxo-2H-benzo[b][1,4]oxazin-4(3H,4aH,5H,6H,7H,8H,8aH)-yl)piperidin-1-yl)pyrrolidine-1-carboxylate (1.098 g) was prepared from tert-butyl 3-(4-((1S,2S)-hydroxycyclohexylamino)piperidin-1-yl)-3-methylpyrrolidine-1-carboxylate (0.994 g, 2.61 mmol). MS: 352.1 (M+1-56).

### Step C: The preparation of of (4aS,8aS)-4-(1-(3-methylpyrrolidin-3-yl)piperidin-4-yl)hexahydro-2H-benzo[b][1,4]oxazin-3(4H)-one hydrochloride

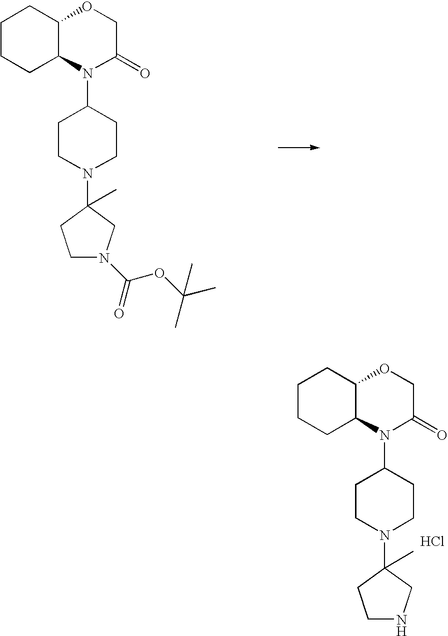

Following the similar procedure described in Example 13, Step D: 4aS,8aS)-4-(1-(3-methylpyrrolidin-3-yl)piperidin-4-yl)hexahydro-2H-benzo[b][1,4]oxazin-3(4H)-one hydrochloride was prepared from tert-butyl 3-methyl-3-(4-((4aS,8aS)-3-oxo-2H-benzo[b][1,4]oxazin-4(3H,4aH,5H,6H,7H,8H,8aH)-yl)piperidin-1-yl)pyrrolidine-1-carboxylate.

### Step D. The preparation of ethyl 3-methyl-3-(4-((4aS,8aS)-3-oxo-2H-benzo[b][1,4]oxazin-4(3H,4aH,5H,6H,7H,8H,8aH)-yl)piperidin-1-yl)pyrrolidine-1-carboxylate (Diastereoisomeric mixtures)

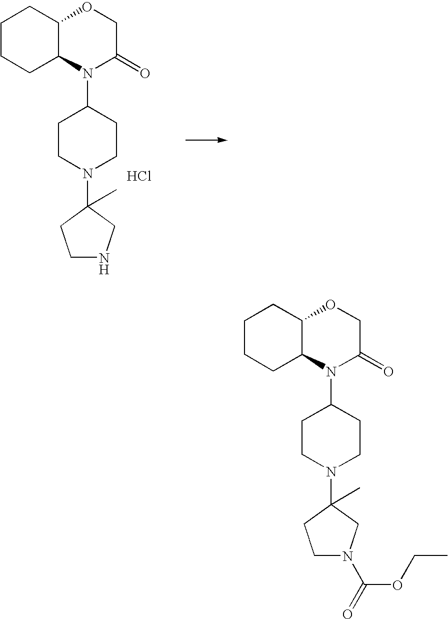

Following the similar procedure of Example 13, Step E: ethyl 3-methyl-3-(4-((4aS,8aS)-3-oxo-2H-benzo[b][1,4]oxazin-4(3H,4aH,5H,6H,7H,8H,8aH)-yl)piperidin-1-carboxylate (Diastereoisomeric mixtures) was prepared from (4aS,8aS)-4-(1-(3-methylpyrrolidin-3-yl)piperidin-4-yl)hexahydro-2H-benzo[b][1,4]oxazin-3(4H)-one hydrochloride.

1H NMR (400 MHz, CHLOROFORM-d) δ ppm 1.14-1.23 (m, 2H) 1.25 (q, J=6.77 Hz, 3H) 1.30-1.51 (m, 6H) 1.59 (br. s., 3H) 1.82 (d, J=10.55 Hz, 2H) 1.92 (d, J=8.20 Hz, 3H), 2.03 (d, J=12.89 Hz, 2H) 2.85 (d, J=14.45 Hz, 1H) 2.93-3.19 (m, 4H) 3.19-3.33 (m, 1H), 3.33-3.48 (m, 1H) 3.48-3.58 (m, 1H) 3.58-3.68 (m, 1H) 3.68-3.82 (m, 1H) 4.02-4.21 (m, 3H), 4.18-4.34 (m, 2H) 4.66-4.82 (m, 2H). HRMS calcd for C21H36N3O4 [M+H]+ 394.27003, found 394.26948.

### Step E. Separation of diastereoisomeric mixture of ethyl 3-methyl-3-(4-((4aS,8aS)-3-oxo-2H-benzo[b][1,4]oxazin-4(3H,4aH,5H,6H,7H,8H,8aH)-yl)piperidin-1-yl)pyrrolidine-1-carboxylate

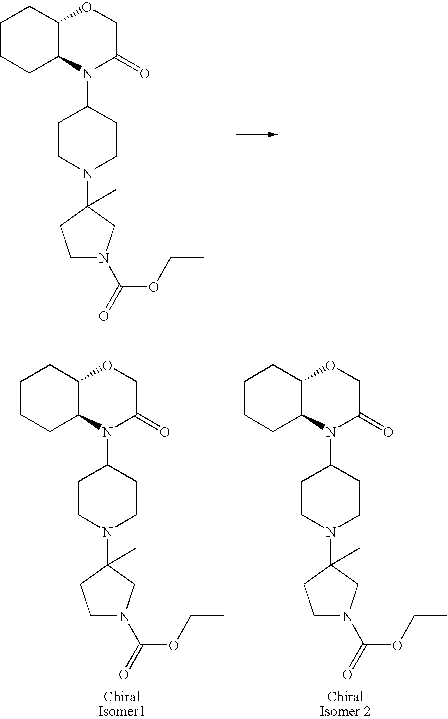

The diastereoisomeric mixture of ethyl 3-methyl-3-(4-((4aS,8aS)-3-oxo-2H-benzo[b][1,4]oxazin-4(3H,4aH,5H,6H,7H,8H,8aH)-yl)piperidin-1-yl)pyrrolidine-1-carboxylate (0.120 g, 0.30 mmol) was separated by chiral SFC (AD Column with IPA+0.1% DEA Iso at 35%, 215 nm, 10 ml/min, column temperature set at 35° C., 30 ul injection volume) to afford the two diastereoisomers:

Isomer 1 (Example 18): ethyl 3-methyl-3-(4-((4aS,8aS)-3-oxo-2H-benzo[b][1,4]oxazin-4(3H,4aH,5H,6H,7H,8H,8aH)-yl)piperidin-1-yl)pyrrolidine-1-carboxylate (Isomer 1) (0.020 g, 33.3%); SFC (AD column): retention time 3.01 min. 1H NMR (400 MHz, CHLOROFORM-d) δ ppm 0.86-1.06 (m, 2H) 1.06-1.15 (m, 2H) 1.18 (t, J=7.23 Hz, 3H) 1.21-1.29 (m, 2H) 1.29-1.45 (m, 2H) 1.43-1.90 (m, 6H) 1.90-2.00 (m, 1H) 2.10 (br. s., 2H), 2.19-2.41 (m, 2H) 2.52-2.85 (m, 2H) 3.02-3.25 (m, 3H) 3.23-3.35 (m, 1H) 3.39 (d, J=6.25 Hz, 1H) 3.42-3.60 (m, 1H) 3.82 (br. s., 1H) 3.97-4.09 (m, 2H) 4.09-4.23 (m, 2H). HRMS calcd for C21H36N3O4 [M+H]+ 394.27003, found 394.26978.

Isomer 2 (Example 19): ethyl 3-methyl-3-(4-((4aS,8aS)-3-oxo-2H-benzo[b][1,4]oxazin-4(3H,4aH,5H,6H,7H,8H,8aH)-yl)piperidin-1-yl)pyrrolidine-1-carboxylate (Isomer 2) (0.050 g, 83%); SFC (AD column): retension time 3.54 min. 1H NMR (400 MHz, CHLOROFORM-d) δ ppm 0.85-1.02 (m, 2H) 1.02-1.12 (m, 2H) 1.03-1.04 (m, 1H) 1.16 (t, J=7.23 Hz, 3H) 1.23 (d, J=15.23 Hz, 2H) 1.27-1.43 (m, 2H) 1.45-1.88 (m, 6H) 1.89-2.00 (m, 2H), 2.00-2.26 (m, 2H) 2.37 (br. s., 2H) 2.81 (br. s., 1H) 3.05-3.24 (m, 3H) 3.32 (d, J=10.55 Hz, 1H) 3.51 (d, J=17.97 Hz, 1H) 3.85 (br. s., 1H) 3.96-4.09 (m, 2H) 4.09-4.22 (m, 2H). HRMS calcd for C21H36N3O4 [M+H]+ 394.27003, found 394.26957.

### Preparation of ethyl 4-methyl-4-(4-oxo-1-piperidyl)piperidine-1-carboxylate

### Step A. The preparation of ethyl 4-cyano-4-(4-hydroxy-1-piperidyl)piperidine-1-carboxylate

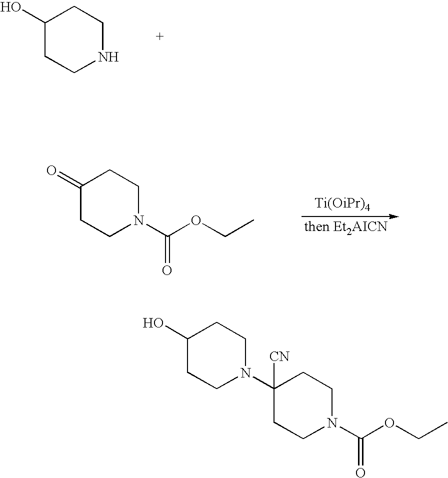

A stirred solution of 4-hydroxypiperidine (1.01 g, 10.0 mmol) and ethyl 4-oxopiperidine-1-carboxylate (1.71 g, 10.0 mmol) in 1,2-dichloroethane (25 mL) was added with titanium isopropoxide (2.3 mL, 11.0 mmol). The reaction mixture was stirred at room temperature for 18 hours. Then a 1.0 M solution of diethylaluminum cyanide (24.0 mL, 24.0 mmol) was added at room temperature and stirred at room temperature for 24 hours. The reaction mixture was diluted with EtOAc and quenched at 0° C. with aqueous saturated NaHCO₃ (10 mL). The mixture was further stirred for 2 hours. The mixture was then filtered through Celite and the filtrate was concentrated in vacuo. The residue was purified by flash chromatography (ethyl acetate/hexane) to afford the title compound (2.45 g, 87%) as oil. 1H NMR (400 MHz, CHLOROFORM-D) δ ppm 1.19 (t, J=7.08 Hz, 3H), 1.45-1.67 (m, 4H), 1.85 (d, J=10.16 Hz, 2H), 2.00 (d, J=12.89 Hz, 2H), 2.20-2.28 (m, 2H), 2.81-2.92 (m, 2H), 3.04-3.23 (m, 3H), 3.58-3.71 (m, 1H), 3.81-3.98 (m, 2H), 4.06 (q, J=7.08 Hz, 2H).

### Step B. The preparation of ethyl 4-(4-hydroxy-1-piperidyl)-4-methyl-piperidine-1-carboxylate

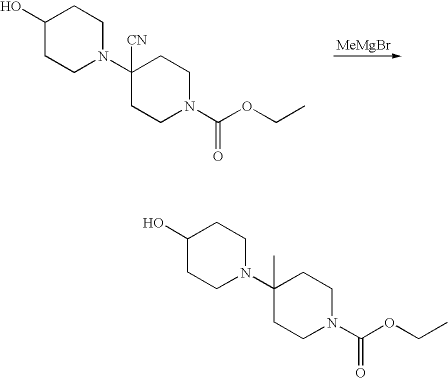

A stirred solution of ethyl 4-cyano-4-(4-hydroxy-1-piperidyl)piperidine-1-carboxylate (2.45 g, 8.69 mmol) in THF (20 mL) was with added a 1.4 M solution of MeMgBr in toluene/THF (18.6 mL, 26.1 mmol) at 0° C. The reaction mixture was stirred at room temperature for 12 hours. The reaction was then quenched with saturated aqueous ammonium chloride, and the mixture was extracted with dichloromethane (2×25 mL). The combined extracts were concentrated in vacuo to afford the title compound (1.54 g, 65%), which was used in the next step without further purification. MS (M+1): 271.26.

### Step C. The preparation of ethyl 4-methyl-4-(4-oxo-1-piperidyl)piperidine-1-carboxylate

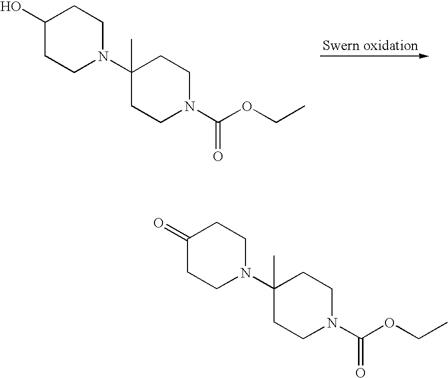

A solution of oxalyl chloride in dichloromethane (2M, 2.05 mL, 4.1 mmol) was cooled to −78° C. under nitrogen atmosphere and was added to a solution of dimethylsulfoxide (0.58 mL, 8.1 mmol) in dichloromethane (6 mL) at −78° C. under nitrogen atmosphere via cannula. After 10 minutes, a solution of ethyl 4-(4-hydroxy-1-piperidyl)-4-methyl-piperidine-1-carboxylate (2.7 mmol) in dichloromethane (3 mL) was added at −78° C. under nitrogen atmosphere to the reaction mixture via cannula. The mixture was stirred at −78° C. for 10 minutes and then triethylamine (1.51 mL, 10.8 mmol) was added dropwise. The reaction was stirred at −78° C. under nitrogen atmosphere for another 20 minutes, and then allowed to warm up to 0° C. over 1 hour. The reaction was quenched with water (10 mL) and diluted with dichloromethane (30 mL). The phases were separated and the aqueous phase was extracted with dichloromethane (2×25 mL). The combined organic phases were washed with saturated aqueous ammonium chloride, brine and dried over Na₂SO₄. The solvent was removed in vacuo to afford the title compound as yellow oil (672 mg, 93%), which was used for the subsequent step without further purification. 1H NMR (400 MHz, CHLOROFORM-D) δ ppm 0.96 (s, 3H), 1.24-1.30 (m, 3H), 1.39-1.53 (m, 2H), 1.72-1.92 (m, 2H), 2.11-2.30 (m, 1H), 2.42 (t, J=5.86 Hz, 2H), 2.51 (t, J=6.05 Hz, 1H), 2.81 (t, J=5.86 Hz, 2H), 2.97 (t, J=6.05 Hz, 1H), 3.22 (t, J=12.01 Hz, 1H), 3.35-3.47 (m, 2H), 3.53-3.72 (m, 2H), 4.14 (q, J=7.10 Hz, 2H). MS (M+1): 269.24.

### Preparation of tert-butyl 4-methyl-4-(4-oxo-1-piperidyl)piperidine-1-carboxylate

### Step A. The preparation of tert-butyl 4-cyano-4-(4-hydroxy-1-piperidyl)piperidine-1-carboxylate

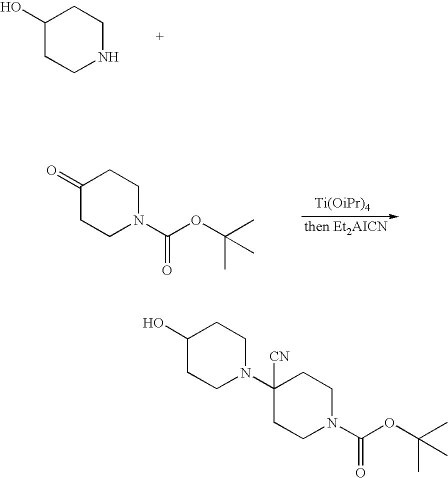

A stirred solution of 4-hydroxypiperidine (2.02 g, 20.0 mmol) and tert-butyl 4-oxopiperidine-1-carboxylate (3.99 g, 20.0 mmol) in 1,2-dichloroethane (50 mL) was added with titanium isopropoxide (4.6 mL, 22.0 mmol). The reaction mixture was stirred at room temperature for 18 hours. A solution of diethylaluminum cyanide in toluene (1M, 48.0 mL, 48.0 mmol) was added and stirred at room temperature for 24 hours. The reaction mixture was diluted with EtOAc and quenched at 0° C. with saturated aqueous NaHCO₃ (20 mL). The mixture was stirred a further 2 hours, filtered through Celite, and the filtrate was concentrated in vacuo to afford the title compound (5.89 g, 95%) as white solid, which was used for the next step without further purification.

### Step B. The preparation of tert-butyl 4-(4-hydroxy-1-piperidyl)-4-methyl-piperidine-1-carboxylate

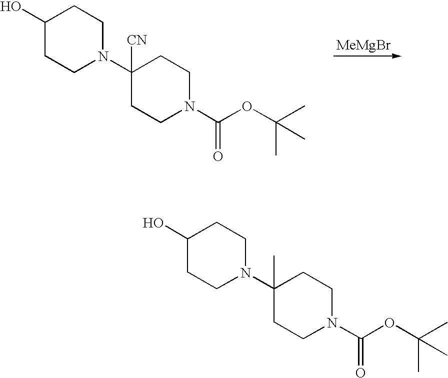

A stirred solution of tert-butyl 4-cyano-4-(4-hydroxy-1-piperidyl)piperidine-1-carboxylate (5.8 g, 18.74 mmol) in THF (40 mL) was added with a 1.4 M solution of MeMgBr in toluene/THF (26.8 mL, 37.48 mmol) at 0° C. The reaction mixture was stirred at room temperature for 12 hours. The reaction was then quenched with saturated aqueous ammonium chloride and the mixture was extracted with dichloromethane (2×30 mL). The combined extracts were concentrated in vacuo to afford the title compound (5.42 g, 97%), which was used for the next step without further purification. MS (M+1): 299.24.

### Step C. The preparation of tert-butyl 4-methyl-4-(4-oxo-1-piperidyl)piperidine-1-carboxylate

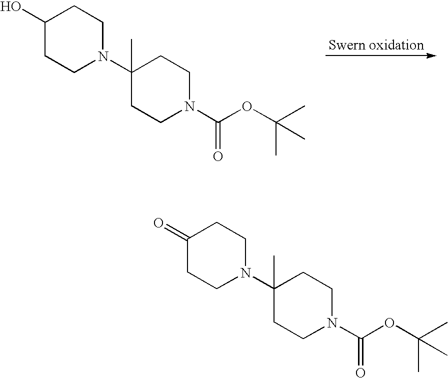

A solution of oxalyl chloride in dichloromethane (2M, 13.67 mL, 27.33 mmol) was cooled to −78° C. under nitrogen atmosphere and was added to a solution of dimethylsulfoxide (3.87 mL, 54.0 mmol) in dichloromethane (40 mL) at −78° C. under nitrogen atmosphere via cannula. After 10 minutes, a solution of tert-butyl 4-(4-hydroxy-1-piperidyl)-4-methyl-piperidine-1-carboxylate (18.0 mmol) in dichloromethane (20 mL) was added at −78° C. under nitrogen atmosphere to the reaction mixture via cannula. The mixture was stirred at −78° C. for 10 minutes and then triethylamine (10.07 mL, 72.0 mmol) was added dropwise. The reaction was stirred at −78° C. under nitrogen atmosphere for another 20 minutes, and then allowed to warm up to 0° C. over 1 hour. The reaction was quenched with water (50 mL) and diluted with dichloromethane (100 mL). The phases were separated and the aqueous phase was extracted with dichloromethane (2×50 mL). The combined organic phases were washed with saturated aqueous ammonium chloride, brine, dried over Na₂SO₄ and concentrated in vacuo to afford the title compound as yellow oil (5.02 g, 94%), which was used for the subsequent step without further purification. MS (M+1): 297.24.

### Preparation of ethyl 3-methyl-3-(4-oxo-1-piperidyl)pyrrolidine-1-carboxylate

### Step A. The preparation of ethyl 3-cyano-3-(4-hydroxy-1-piperidyl)pyrrolidine-1-carboxylate

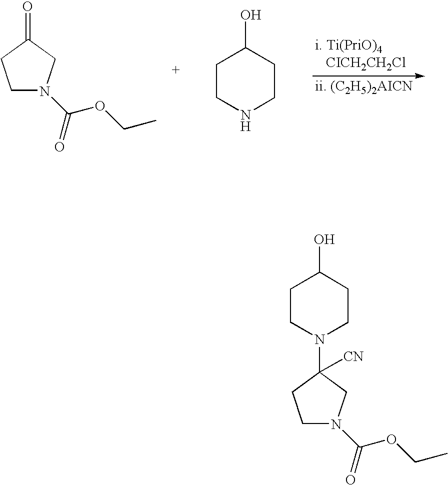

A stirred solution of 4-hydroxypiperidine (464 mg, 4.58 mmol) and ethyl 3-oxopyrrolidine-1-carboxylate (610 mg, 3.82 mmol) in 1,2-dichloroethane (25 mL) was added with titanium isopropoxide (1.09 g, 3.82 mmol), and the mixture was stirred at room temperature overnight. Then a 1.0 M solution of diethylaluminum cyanide (1.02 g, 9.17 mmol) was added at room temperature and the mixture was stirred for 24 hours. The reaction mixture was diluted with dichloromethane (25 mL) and quenched with saturated ammonium chloride solution (10 mL) at 0° C. Then mixture was filtered through a small pad of celite, and the filtrate was concentrated in vacuo to afford the title compound as a yellow gum (1.0 g). ¹H NMR (CDCl₃, 400 MHz): δ 4.22 (q, 2H), 4.21-4.1 (dd, 1H), 3.79-3.62 (m, 3H), 3.38 (dd, 1H), 2.9 (brs, 1H), 2.7 (brs, 1H), 2.54-2.35 (m, 3H), 2.18-1.85 (brm, 3H), 1.68-1.45 (m, 3H), 1.25 (t, 3H). MS (M+1): 268.14.

### Step B. The preparation of ethyl 3-(4-hydroxy-1-piperidyl)-3-methyl-pyrrolidine-1-carboxylate

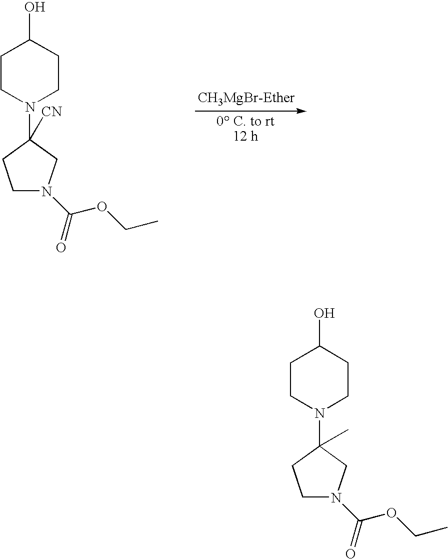

A stirred solution of ethyl 3-cyano-3-(4-hydroxy-1-piperidyl)pyrrolidine-1-carboxylate (1.0 gm, 3.74 mmol) in tetrahydrofuran (25 mL) was added with a 1.4 M solution of methyl magnesium bromide in toluene/THF (5.35 mL, 7.48 mmol) at 0° C., and the mixture was allowed to warm to room temperature. The mixture was stirred for another 12 hours at room temperature. The reaction was quenched with saturated aqueous ammonium chloride solution (5 mL) at 0° C. and diluted with ethyl acetate (25 mL). The phases were separated and the organic phase was washed with brine, dried over anhydrous Na₂SO₄. The solvent was removed in vacuo to afford the title compound as a pale solid (830 mg), which was used for the subsequent step without further purification. MS (M+1): 257.16.

### Step C. The preparation of ethyl 3-methyl-3-(4-oxo-1-piperidyl)pyrrolidine-1-carboxylate

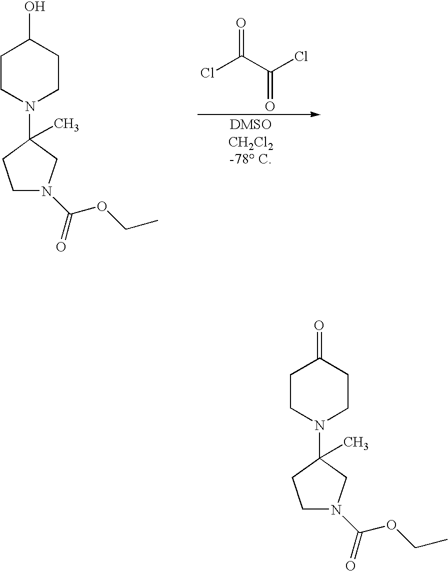

2M Oxalyl chloride solution in dichloromethane (617 mg, 4.86 mmol) was taken into a oven dried round bottom flak and cooled to −78° C. under nitrogen atmosphere. Then dimethyl sulfoxide (767 mg, 9.72 mmol) in anhydrous dichloromethane (5 mL) was added dropwise. After 10 minutes, a solution of ethyl 3-(4-hydroxy-1-piperidyl)-3-methyl-pyrrolidine-1-carboxylate (830 mg, 3.24 mmol) in dichloromethane (10 mL) was cannulated into the flask and stirred at −78° C. for another 10 minutes. Triethylamine (1.31 g, 12.96 mmol) was then added and stirred at −78° C. for 30 minutes, allowed to warm to 0° C. over 30 minutes and was quenched with saturated solution of ammonium chloride (10 mL). The product was extracted into dichloromethane (2×50 mL) and the combined organic phases were washed with brine, dried over anhydrous Na₂SO₄. The solvent was removed in vacuo to afford the title compound as a yellow oil (810 mg, 90%). 1H NMR (CDCl₃, 400 MHz): δ 4.18 (m, 2H), 3.88 (m, 1H), 3.62-3.35 (m, 3H), 2.92 (m, 1H), 2.85 (brs, 2H), 2.75 (brs, 1H), 2.4-2.39 (m, 4H), 2.05-1.89 (m, 1H), 1.41 (m, 1H), 1.26 (t, 3H), 1.08 (s, 3H) MS (M+1): 255.12.

### Preparation of tert-butyl 3-methyl -3-(4-oxopiperidin-1-yl)pyrrolidine-1-carboxylate

### Step A: Preparation of tert-butyl 3-cyano-3-(4-hydroxypiperidin-1-yl)pyrrolidine-1-carboxylate

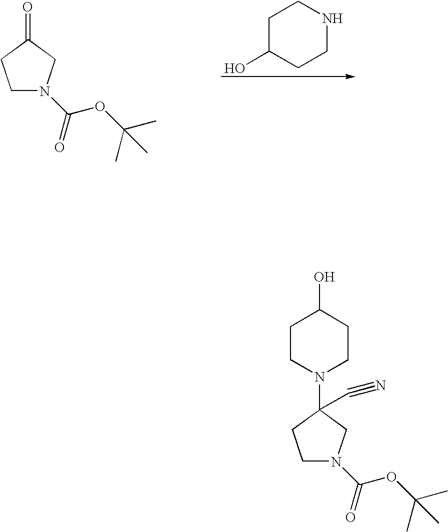

To a mixture of piperidin-4-ol (5.06 g, 0.05 mol) and tert-butyl 3-oxopyrrolidine-1-carboxylate (7.72 g, 0.04 mol) in ClCH₂CH₂Cl (200 mL) was added tetraisopropoxytitanium (0.012 kg, 0.04 mol). The reaction mixture was stirred at room temperature for 24 hours. 1M solution of cyanodiethylaluminum (100 mL, 0.10 mol) in toluene was added and the mixture was stirred at room temperature for 24 hours. The solution was then diluted with dichloromethane (250 mL) and quenched with saturated aqueous NH₄Cl solution (100 mL) at 0° C. The mixture was filtered through a small pad of celite, and the filtrate was concentrated in vacuo to give the title product as pale yellow solid, which was used in the subsequent step without further purification. 1H NMR (400 MHz, CHLOROFORM-D) δ ppm 1.47 (s, 9H) 1.55-1.70 (m, 4H) 1.87-2.12 (m, 3H) 2.29-2.53 (m, 3H) 2.65-2.77 (m, 1H) 2.88 (d, J=8.59 Hz, 1H) 3.28 (d, J=9.37 Hz, 1H) 3.48-3.84 (m, 2H) 3.99 (dd, J=42.77, 10.74 Hz, 1H).

### Step B: Preparation of tert-butyl 3-(4-hydroxypiperidin-1-yl)-3-methylpyrrolidine-1-carboxylate

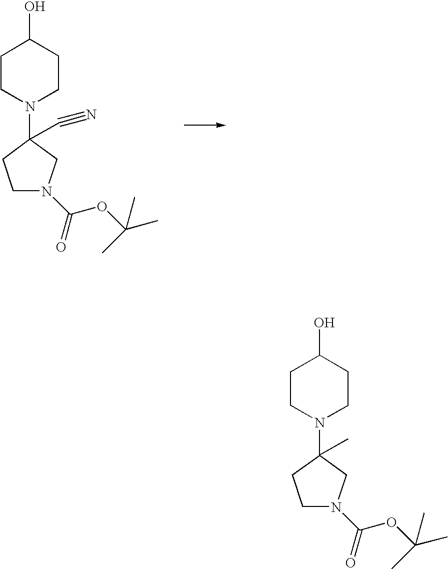

To a solution of tert-butyl 3-cyano-3-(4-hydroxypiperidin-1-yl)pyrrolidine-1-carboxylate (1 g, 3.39 mmol) in dry THF (20 mL) and was added a 1.0 M solution of methylmagnesium bromide (13.5 mL, 13.54 mmol) in butylether at 0° C. The reaction mixture was stirred at room temperature for 4 hours. The reaction mixture was quenched with saturated aqueous NH₄Cl solution (30 mL) at 0° C. and diluted with ethyl acetate (50 mL). The layers were separated and the organic layer was washed with brine, dried over Na₂SO₄, filtered and filtrate was concentrated in vacuo to give the title compound (1.069 g), which was used in the subsequent step without further purification.

### Step C: Preparation of tert-butyl 3-methyl-3-(4-oxopiperidin-1-yl)pyrrolidine-1-carboxylate

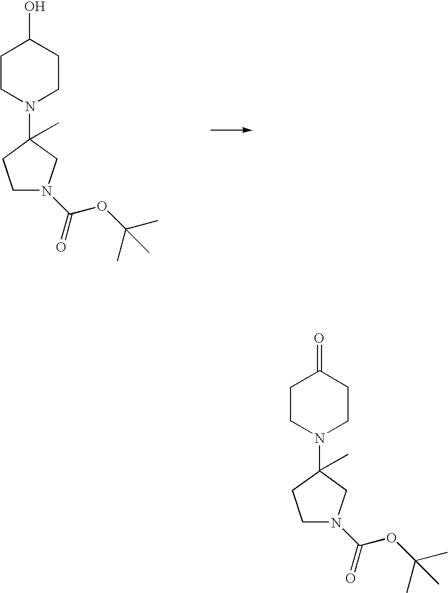

To a solution of oxalyl dichloride (2M, 2.5 mL, 5.09 mmol) in dichloromethane was added dropwise DMSO (0.722 mL, 10.17 mmol) at −78° C. under an nitrogen atmosphere. The reaction flask was kept in a −78° C. bath and after stirring for 10 minutes, a solution of tert-butyl 3-(4-hydroxypiperidin-1-yl)-3-methylpyrrolidine-1-carboxylate (0.964 g, 3.39 mmol) in dichloromethane (2 mL) was added and stirred for another 10 minutes. Triethylamine (1.890 mL, 13.56 mmol) was added and stirred at −78° C. for 30 minutes and then the reaction mixture was allowed to warm to 0° C. over 30 minutes. The reaction was quenched with saturated aqueous NH₄Cl (10 mL) and extracted with dichloromethane (3×10 mL). The combined the organic extract was washed with brine, dried over MgSO₄, filtered and concentrated in vacuo to give the title compound (0.856 g, 89%) as pale yellow solid, which was used in the subsequent step without further purification.

Various modifications of the invention, in addition to those described herein, will be apparent to those skilled in the art from the foregoing description. Such modifications are also intended to fall within the scope of the appended claims. Each reference, including all patent, patent applications, publications, and gene bank sequences cited in the present application, is incorporated herein by reference in its entirety.

1. A compound of Formula I: 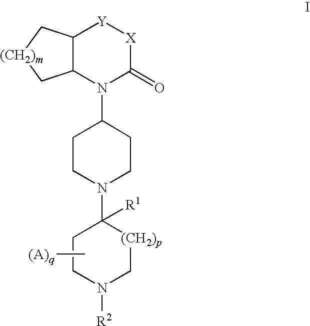
or pharmaceutically acceptable salt thereof;
wherein:
Y is —CR³R⁴—, —NR⁵—, —O—, or —S—;
X is —CR⁶R⁷—, —NR⁸—, —O—, or —S—;
with the proviso that either Y is —CR³R⁴— or X is —CR⁶R⁷—;
each A is, independently, C₁₋₃ alkyl, or two A linked together to form a C₁₋₃alkylene bridge;
R¹ is hydrogen, C₁₋₆ alkyl, or C₁₋₆ haloalkyl;
R² is —C(O)OR^{a}, —C(O)R^{b}, —C(O)NR^{c}R^{d}, C₁₋₆ alkyl, C₁₋₆ haloalkyl, C₃₋₇ cycloalkyl, C₃₋₇ cycloalkyl-C₁₋₃alkyl C₃₋₇ heterocycloalkyl, C₃₋₇ heterocycloalkyl-C₁₋₃ alkyl, C₆₋₁₀aryl-C₁₋₃alkyl, C₃₋₉ heteroaryl, or C₃₋₉heteroaryl-C₁₋₃alkyl; wherein the rings of said C₆₋₁₀aryl, C₆₋₁₀aryl-C₁₋₃alkyl, C₃₋₉ heteroaryl, and C₃₋₉heteroaryl-C₁₋₃ alkyl are each optionally substituted by 1, 2, 3, or 4 independently selected R⁹ groups; wherein the rings of said C₃₋₇ cycloalkyl, C₃₋₇ cycloalkyl-C₁₋₃ alkyl, C₃₋₇ heterocycloalkyl, and C₃₋₇ heterocycloalkyl-C₁₋₃ alkyl are each optionally substituted by 1, 2, 3, or 4 independently selected R¹⁰ groups; and wherein said C₁₋₆ alkyl, C₂₋₆ alkenyl, C₂₋₆ alkynyl, C₁₋₆ haloalkyl, C₁₋₆ alkoxy, and C₁₋₆ haloalkoxy are each optionally substituted by 1, 2, or 3 independently selected R¹¹ groups;
R³, R⁴, R⁶, and R⁷ are each, independently, hydrogen, fluoro, C₁₋₄ alkyl, C₁₋₄ alkoxymethyl, cyanoC₁₋₄ alkyl or C₁₋₄ haloalkyl;
R⁵ and R⁸ are each, independently, hydrogen, C₁₋₄ alkyl, or C₁₋₄ haloalkyl;
each R⁹ and R¹⁰ is, independently, phenyl, C₃₋₆ cycloalkyl, C₂₋₅ heterocycloalkyl, C₃₋₅ heteroaryl, —CN, —SR^{e}, —OR^{e}, —O(CH₂)ᵣ—OR^{e}, R^{e}, —C(O)—R^{e}, —CO₂R^{e}, —SO₂R^{e}, —SO₂NR^{e}R^{f}, halogen, —NO₂, —NR^{e}R^{f}, —(CH₂)ᵣNR^{e}R^{f}, or —C(O)—NR^{e}R^{f};
each R¹¹ is, independently, —CN, —NO₂, —OR^{e}, or —NR^{e}R^{f};
R^{a}, R^{b}, R^{c}, and R^{d} are each, independently, hydrogen, C₁₋₇ alkyl, C₂₋₆ alkenyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the rings of said C₆₋₁₀aryl, C₆₋₁₀aryl-C₁₋₃alkyl, C₃₋₉ heteroaryl, and C₃₋₉ heteroaryl-C₁₋₃alkyl are each optionally substituted by 1, 2, 3, or 4 independently selected R¹² groups; wherein the rings of said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl, are each optionally substituted by 1, 2, 3, or 4 independently selected R¹³ groups; and wherein said C₁₋₇ alkyl, C₂₋₆ alkenyl, C₂₋₆ alkynyl, C₁₋₆ haloalkyl, C₁₋₇ alkoxy, and C₁₋₆ haloalkoxy are each optionally substituted by 1, 2, or 3 independently selected R¹⁴ groups;
each R¹², R¹³, and R¹⁴ is, independently, phenyl, C₃₋₆ cycloalkyl, C₂₋₅ heterocycloalkyl, C₃₋₅ heteroaryl, —CN, —SR^{g}, —OR^{g}, —O(CH₂)ᵣ—OR^{g}, R^{g}, —C(O)—R^{g}, —CO₂R^{g}, —SO₂R^{g}, —SO₂NR^{g}R^{h}, halogen, —NO₂, —NR^{g}R^{h}, —(CH₂)ᵣNR^{g}R^{h}, or —C(O)—NR^{g}R^{h};
each R^{e}, R^{f}, R^{g} and R^{h} is, independently hydrogen, C₁₋₆ alkyl, C₂₋₆ alkenyl or C₁₋₆ haloalkyl,
m is 1, 2, or 3;
p is 0, 1, or 2;
q is an integer from 0 to [6+(p×2)]; and
r is 1, 2, 3 or 4;
with the proviso that the compound is not isopropyl 4′-methyl-4-((4aS,8aS)-2-oxooctahydroquinoxalin-1(2H)-yl)-1,4′-bipiperidine-1′-carboxylate,
isopropyl 4-[4-[(4aS,8aS)-2-oxo-3,4,4a,5,6,7,8,8a-octahydroquinoxalin-1-yl]-1-piperidyl]-4-methyl-piperidine-1-carboxylate,
isopropyl (3S)-3-[4-[(4aS,8aS)-3-oxo-4a,5,6,7,8,8a-hexahydrobenzo[b][1,4]oxazine-4-yl]-1-piperidyl]pyrrolidine-1-carboxylate,
tert-butyl 4-[4-[(4aR,8aR)-2-oxo-4a,5,6,7,8,8a-hexahydro-4H-benzo[d][1,3]oxazin-1-yl]-1-piperidyl]piperidine-1-carboxylate,
isopropyl 4-[4-[(4aS,8aS)-3-oxo-4a,5,6,7,8,8a-hexahydrobenzo[b][1,4]oxazin-4-yl]-1-piperidyl]-4-methyl-piperidine-1-carboxylate,
(4aS,8aS)-1-[1-[1-(2-methylbenzoyl)-4-piperidyl]-4-piperidyl]-4a,5,6,7,8,8a-hexahydro-4H-benzo[d][1,3]oxazin-2-one,
tert-butyl 4-[4-[(4aS,8aS)-3-oxo-4a,5,6,7,8,8a-hexahydrobenzo[b][1,4]oxazin-4-yl]-1-piperidyl]piperidine-1-carboxylate,
methyl 4-[4-[(4aS,8aS)-2-oxo-4a,5,6,7,8,8a-hexahydro-4H-benzo[d][1,3]oxazin-1-yl]-1-piperidyl]piperidine-1-carboxylate, or pharmaceutically acceptable salt thereof.

2. The compound according to claim 1, or pharmaceutically acceptable salt thereof, wherein:
Y is —CR³R⁴—, —NR⁵—, or —O—; and
X is —CR⁶R⁷—, —NR⁸—, or —O—.

3. The compound according to claim 1, or pharmaceutically acceptable salt thereof, wherein:
Y is —CR³R⁴—, or —O—; and
X is —CR⁶R⁷—, —NR⁸—, or —O—.

4. The compound according to claim 1, or pharmaceutically acceptable salt thereof, wherein R¹ is hydrogen, C₁₋₆ alkyl, or fluorinated C₁₋₆ haloalkyl;

5. The compound according to claim 1, or pharmaceutically acceptable salt thereof, wherein R¹ is hydrogen, methyl, ethyl, fluoromethyl, difluoromethyl, or trifluoromethyl.

6. The compound according to claim 1, or pharmaceutically acceptable salt thereof, wherein R¹ is hydrogen or methyl.

7. The compound according to claim 1, or pharmaceutically acceptable salt thereof, wherein R² is —C(O)OR^{a}, —C(O)R^{b}, —C(O)NR^{c}R^{d}, C₃₋₇ cycloalkyl-C₁₋₃alkyl, C₃₋₇ heterocycloalkyl-C₁₋₃ alkyl, C₆₋₁₀ aryl-C₁₋₃alkyl, or C₃₋₉ heteroaryl-C₁₋₃alkyl; wherein said C₆₋₁₀ aryl-C₁₋₃alkyl and C₃₋₉ heteroaryl-C₁₋₃alkyl are each optionally substituted by 1, 2, 3, or 4 independently selected R⁹ groups; and wherein said C₃₋₇ cycloalkyl-C₁₋₃alkyl and C₃₋₇ heterocycloalkyl-C₁₋₃ alkyl are each optionally substituted by 1, 2, 3, or 4 independently selected R¹⁰ groups.

8. The compound according to claim 1, or pharmaceutically acceptable salt thereof, wherein R² is —C(O)OR^{a}, —C(O)R^{b}, —C(O)NR^{c}R^{d}, —CH₂—C₃₋₇ cycloalkyl, —CH₂—C₃₋₇ heterocycloalkyl, —CH₂—C₆₋₁₀aryl, or —CH₂—C₆₋₉heteroaryl; wherein the rings of said —CH₂—C₆₋₁₀aryl and —CH₂—C₆₋₉heteroaryl are each optionally substituted by 1, 2, 3, or 4 independently selected R^{g} groups; and wherein the rings of said —CH₂—C₃₋₇ cycloalkyl and —CH₂—C₃₋₇ heterocycloalkyl, are each optionally substituted by 1, 2, 3, or 4 independently selected R¹⁰ groups.

9. The compound according to claim 1, or pharmaceutically acceptable salt thereof, wherein R² is —C(O)OR^{a}, —C(O)R^{b}, —C(O)NR^{c}R^{d}, C₆₋₁₀aryl-C₁₋₃alkyl or C₃₋₉heteroaryl-C₁₋₃alkyl; wherein the rings of said C₆₋₁₀aryl-C₁₋₃alkyl and C₃₋₉heteroaryl-C₁₋₃alkyl are each optionally substituted by 1, 2, or 3 independently selected R⁹ groups.

10. The compound according to claim 1, or pharmaceutically acceptable salt thereof, wherein R² is —C(O)OR^{a}, —C(O)R^{b}, —C(O)NR^{c}R^{d}, —CH₂—C₆₋₁₀aryl, or —CH₂—C₆₋₉heteroaryl; wherein the rings of said —CH₂—C₆₋₁₀aryl and —CH₂—C₆₋₉heteroaryl are each optionally substituted by 1, 2, 3, or 4 independently selected R⁹ groups.

11. The compound according to claim 1, or pharmaceutically acceptable salt thereof, wherein R² is —C(O)OR^{a} or —C(O)R^{b}.

12. The compound according to claim 1, or pharmaceutically acceptable salt thereof, wherein R³, R⁴, R⁶, and R⁷ are each, independently, hydrogen or C₁₋₄ alkyl.

13. The compound according to claim 1, or pharmaceutically acceptable salt thereof, wherein R³, R⁴, R⁶, and R⁷ are hydrogen.

14. The compound according to claim 1, or pharmaceutically acceptable salt thereof, wherein R⁵ and R⁸ are each, independently, hydrogen or C₁₋₄ alkyl.

15. The compound according to claim 1, or pharmaceutically acceptable salt thereof, wherein R⁵ and R⁸ are each, independently, hydrogen or methyl.

16. The compound according to claim 1, or pharmaceutically acceptable salt thereof, wherein R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀ aryl, C₆₋₁₀ aryl-C₁₋₃ alkyl, C₃₋₉ heteroaryl, or C₃₋₉ heteroaryl-C₁₋₃alkyl; wherein the rings of said C₆₋₁₀ aryl, C₆₋₁₀ aryl-C₁₋₃alkyl, C₃₋₉ heteroaryl, and C₃₋₉ heteroaryl-C₁₋₃alkyl are each optionally substituted with 1, 2, or 3 independently selected R¹² groups; and wherein the rings of said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl are each optionally substituted with 1, 2, or 3 independently selected R¹³ groups.

17. The compound according to claim 1, or pharmaceutically acceptable salt thereof, wherein R^{a}, R^{b}, R^{c}, and R^{d} are each, independently, C₁₋₇ alkyl, C₂₋₆ alkynyl, C₁₋₆ haloalkyl, C₃₋₇ cycloalkyl, C₆₋₁₀ aryl, or C₃₋₉ heteroaryl; wherein the rings of said C₆₋₁₀ aryl and C₃₋₉ heteroaryl are each optionally substituted with 1, 2, or 3 independently selected R¹² groups.

18. The compound according to claim 1, or pharmaceutically acceptable salt thereof, wherein R^{a}, R^{b}, R^{c}, and R^{d} are each, independently, C₁₋₇ alkyl, —CH₂—(C₂₋₅ alkynyl), C₁₋₆ haloalkyl, C₃₋₇ cycloalkyl, C₆₋₁₀ aryl, or C₃₋₉ heteroaryl; wherein the rings of said C₆₋₁₀ aryl and C₃₋₉ heteroaryl are each optionally substituted with 1, 2, or 3 independently selected R¹² groups.

19. The compound according to claim 1, or pharmaceutically acceptable salt thereof, wherein R^{a}, R^{b}, R^{c}, and R^{d} are each, independently, C₁₋₇ alkyl, C₁₋₆ haloalkyl, C₃₋₇ cycloalkyl, phenyl, or C₃₋₇ heteroaryl; where rings of said phenyl or C₃₋₉ heteroaryl is each optionally substituted with 1 or 2 independently selected R¹² groups.

20. The compound according to claim 1, or pharmaceutically acceptable salt thereof, wherein R^{a} and R^{b} are each, independently, C₁₋₇ alkyl, C₁₋₆ haloalkyl, C₃₋₇ cycloalkyl, phenyl, or C₃₋₉ heteroaryl; wherein the rings of said phenyl or C₃₋₉ heteroaryl is each optionally substituted with 1 or 2 independently selected R¹² groups.

21. The compound according to claim 1, or pharmaceutically acceptable salt thereof, wherein:
R^{a} is, independently, ethyl, isopropyl, or cyclopropyl; and
R^{b} is, independently, phenyl, pyrrolyl, or thienyl, wherein said phenyl, pyrrolyl or thienyl is optionally substituted with 1 R¹² group.

22. The compound according to claim 1, or pharmaceutically acceptable salt thereof, wherein each R¹² is, independently, halogen, —CN, —NO₂, —OH, C₁₋₆ alkyl C₁₋₆ haloalkyl, C₁₋₆ alkoxy, C₁₋₆ haloalkoxy, —NR^{g}R^{h}, —(CH₂)ᵣNR^{g}R^{h} or —SO₂R^{g}.

23. The compound according to claim 1, or pharmaceutically acceptable salt thereof, wherein each R¹² is, independently, halogen, —CN, —NO₂, —OH, C₁₋₆ alkyl C₁₋₆ haloalkyl, C₁₋₆ alkoxy, C₁₋₆ haloalkoxy, or —NR^{g}R^{h}.

24. The compound according to claim 1, or pharmaceutically acceptable salt thereof, wherein each R¹² is, independently, C₁₋₆ alkyl C₁₋₆ haloalkyl, C₁₋₆ alkoxy, or C₁₋₆ haloalkoxy.

25. The compound according to claim 1, or pharmaceutically acceptable salt thereof, wherein each R¹² is, independently, C₁₋₆ alkyl or C₁₋₆ alkoxy.

26. The compound according to claim 1, or pharmaceutically acceptable salt thereof, wherein each R¹² is, independently, methoxy or methyl.

27. The compound according to claim 1, or pharmaceutically acceptable salt thereof, wherein each R¹³ is, independently, C₁₋₆ alkyl C₁₋₆ haloalkyl, C₁₋₆ alkoxy, or C₁₋₆ haloalkoxy.

28. The compound according to claim 1, or pharmaceutically acceptable salt thereof, wherein each R¹⁴ is, independently, C₁₋₆ alkyl C₁₋₆ haloalkyl, C₁₋₆ alkoxy, or C₁₋₆ haloalkoxy.

29. The compound according to claim 1, or pharmaceutically acceptable salt thereof, wherein each R⁹ is, independently, halogen, —CN, —NO₂, hydroxyl, C₁₋₆ alkyl C₁₋₆ haloalkyl, C₁₋₆ alkoxy, C₁₋₆ haloalkoxy, —NR^{e}R^{f}, —(CH₂)ᵣNR^{e}R^{f} or —SO₂R^{e}.

30. The compound according to claim 1, or pharmaceutically acceptable salt thereof, wherein each R⁹ is, independently, halogen, —CN, —NO₂, —OH, C₁₋₆ alkyl C₁₋₆ haloalkyl, C₁₋₆ alkoxy, or C₁₋₆ haloalkoxy.

31. The compound according to claim 1, or pharmaceutically acceptable salt thereof, wherein each R¹⁰ is, independently, —OH, —CN, —NO₂, hydroxyl, C₁₋₆ alkyl C₁₋₆ haloalkyl, C₁₋₆ alkoxy, C₁₋₆ haloalkoxy, —NR^{e}R^{f}, —(CH₂)ᵣNR^{e}R^{f} or —SO₂R^{e}.

32. The compound according to claim 1, or pharmaceutically acceptable salt thereof, wherein each R¹⁰ is, independently, C₁₋₄ alkyl C₁₋₄ haloalkyl, C₁₋₄ alkoxy, or C₁₋₄ haloalkoxy.

33. The compound according to 1, or pharmaceutically acceptable salt thereof, wherein each A is methyl.

34. The compound according to claim 1, or pharmaceutically acceptable salt thereof, wherein q is 0.

35. The compound according to claim 1, or pharmaceutically acceptable salt thereof, wherein m is 2.

36. The compound according to claim 1, or pharmaceutically acceptable salt thereof, wherein p is 0 or 1.

37-51. (canceled)

52. A compound selected from:
Ethyl 4-[4-[(4aR,8aS)-2-oxo-3,4,4a,5,6,7,8,8a-octahydroquinazolin-1-yl]-1-piperidyl]piperidine-1-carboxylate;
Propan-2-yl 4-[4-[(4aR,8aS)-2-oxo-3,4,4a,5,6,7,8,8a-octahydroquinazolin-1-yl]-1-piperidyl]piperidine-1-carboxylate;
(4aR,8aS)-1-[1-[1-(Cyclopropanecarbonyl)-4-piperidyl]-4-piperidyl]-3,4,4a,5,6,7,8,8a-octahydroquinazolin-2-one;
(4aR,8aS)-1-[1-[1-(2-Methylbenzoyl)-4-piperidyl]-4-piperidyl]-3,4,4a,5,6,7,7,8,8a-octahydroquinazolin-2-one;
Ethyl 3-[4-[(4aR,8aS)-2-oxo-3,4,4a,5,6,7,8,8a-octahydroquinazolin-1-yl]-1-piperidyl]pyrrolidine-1-carboxylate;
Propan-2-yl 4-[4-[(4aR,8aS)-3-methyl-2-oxo-4a,5,6,7,8,8a-hexahydro-4H-quinazolin-1-yl]-1-piperidyl]piperidine-1-carboxylate;
Ethyl 4-[4-[(4aR,8aS)-2-oxo-3,4,4a,5,6,7,8,8a-octahydroquinazolin-1-yl]-1-piperidyl]-4-methyl-piperidine-1-carboxylate;
Propan-2-yl 4-[4-[(4aR,8aS)-2-oxo-3,4,4a,5,6,7,8,8a-octahydroquinazolin-1-yl]-1-piperidyl]-4-methyl-piperidine-1-carboxylate;
Ethyl 4-[4-[(1S,6S)-9-oxo-7-oxa-10-azabicyclo[4.4.0]dec-10-yl]-1-piperidyl]piperidine-1-carboxylate;
Propan-2-yl 4-[4-[(1S,6S)-9-oxo-7-oxa-10-azabicyclo[4.4.0]dec-10-yl]-1-piperidyl]piperidine-1-carboxylate;
(1S,6S)-10-[1-[1-(2-Methylbenzoyl)-4-piperidyl]-4-piperidyl]-7-oxa-10-azabicyclo[4.4.0]decan-9-one;
(1S,6S)-10-[1-[1-(1-Methylpyrrole-2-carbonyl)-4-piperidyl]-4-piperidyl]-7-oxa-10-azabicyclo[4.4.0]decan-9-one;
Ethyl (3S)-3-[4-[(1S,6S)-9-oxo-7-oxa-10-azabicyclo[4.4.0]dec-10-yl]-1-piperidyl]pyrrolidine-1-carboxylate;
Propan-2-yl 4-[4-[(1R,6R)-9-oxo-7-oxa-10-azabicyclo[4.4.0]dec-10-yl]-1-piperidyl]piperidine-1-carboxylate;
Ethyl 4-[4-[(1S,6S)-9-oxo-8-oxa-10-azabicyclo[4.4.0]dec-10-yl]-1-piperidyl]piperidine-1-carboxylate;
Propan-2-yl 4-[4-[(1S,6S)-9-oxo-8-oxa-10-azabicyclo[4.4.0]dec-10-yl]-1-piperidyl]piperidine-1-carboxylate; and
(±)(trans)-10-[1-[1-(3-Methoxythiophene-2-carbonyl)-4-piperidyl]-4-piperidyl]-8-oxa-10-azabicyclo[4.4.0]decan-9-one;
Ethyl 3-methyl-3-(4-((4aS,8aS)-3-oxo-2H-benzo[b][1,4]oxazin-4(3H,4aH,5H,6H,7H,8H,8aH)-yl)piperidin-1-yl)pyrrolidine-1-carboxylate (ISOMER 1);
Ethyl 3-methyl-3-(4-((4aS,8aS)-3-oxo-2H-benzo[b][1,4]oxazin-4(3H,4aH,5H,6H,7H,8H,8aH)-yl)piperidin-1-yl)pyrrolidine-1-carboxylate (ISOMER 2);
and pharmaceutically acceptable salts thereof.

53-56. (canceled)

57. A pharmaceutical composition comprising a compound according to claim 1, or pharmaceutically acceptable salt thereof, and a pharmaceutically acceptable carrier.

58. A method for the therapy of pain in a warm-blooded animal, comprising the step of administering to said animal in need of such therapy a therapeutically effective amount of a compound according to claim 1, or pharmaceutically acceptable salt thereof.

59. A method for the therapy of Alzheimer's disease in a warm-blooded animal, comprising the step of administering to said animal in need of such therapy a therapeutically effective amount of a compound according to claim 1, or pharmaceutically acceptable salt thereof.

60. A method for the therapy of schizophrenia in a warm-blooded animal, comprising the step of administering to said animal in need of such therapy a therapeutically effective amount of a compound according to claim 1, or pharmaceutically acceptable salt thereof.

61. A method for the therapy of anxiety in a warm-blooded animal, comprising the step of administering to said animal in need of such therapy a therapeutically effective amount of a compound according to claim 1, or pharmaceutically acceptable salt thereof.

62. A method for the therapy of depression in a warm-blooded animal, comprising the step of administering to said animal in need of such therapy a therapeutically effective amount of a compound according to claim 1, or pharmaceutically acceptable salt thereof.

63. A process for preparing a compound of claim 1, comprising reacting a compound of Formula IX, or pharmaceutically acceptable salt thereof: 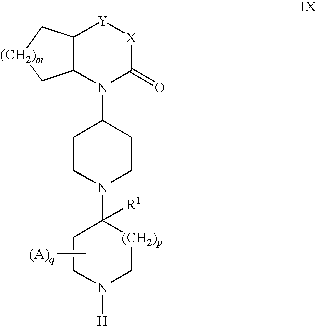
with a compound of Formula R^{a}OC(O)-L¹, or salt thereof, wherein L¹ is halogen, under conditions and for a time sufficient to form a compound of Formula I;
wherein:
Y is —CR³R⁴—, —NR⁵—, —O—, or —S—;
X is —CR⁶R⁷—, —NR⁸—, —O—, or —S—;
with the proviso that either Y is —CR³R⁴— or X is —CR⁶R⁷—;
each A is, independently, C₁₋₃ alkyl, or two A linked together to form a C₁₋₃alkylene bridge;
R¹ is hydrogen, C₁₋₆ alkyl, or C₁₋₆ haloalkyl;
R² is —C(O)OR^{a};
R³, R⁴, R⁶, and R⁷ are each, independently, hydrogen, fluoro, C₁₋₄ alkyl, C₁₋₄ alkoxymethyl, cyanoC₁₋₄ alkyl, or C₁₋₄ haloalkyl;
R⁵ and R⁸ are each, independently, hydrogen, C₁₋₄ alkyl, or C₁₋₄ haloalkyl;
R^{a}, R^{b}, R^{c}, and R^{d} are each, independently, hydrogen, C₁₋₇ alkyl, C₂₋₆ alkenyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the rings of said C₆₋₁₀aryl, C₆₋₁₀aryl-C₁₋₃alkyl, C₃₋₉ heteroaryl, and C₃₋₉ heteroaryl-C₁₋₃alkyl are each optionally substituted by 1, 2, 3, or 4 independently selected R¹² groups; wherein the rings of said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl, are each optionally substituted by 1, 2, 3, or 4 independently selected R¹³ groups; and wherein said C₁₋₇ alkyl, C₂₋₆ alkenyl, C₂₋₆ alkynyl, C₁₋₆ haloalkyl, C₁₋₇ alkoxy, and C₁₋₆ haloalkoxy are each optionally substituted by 1, 2, or 3 independently selected R¹⁴ groups;
each R¹², R¹³, and R¹⁴ is, independently, phenyl, C₃₋₆cycloalkyl, C₂₋₅ heterocycloalkyl, C₃₋₅ heteroaryl, —CN, —SR^{g}, —OR^{g}, —O(CH₂)ᵣ—OR^{g}, R^{g}, —C(O)—R^{g}, —CO₂R^{g}, —SO₂R^{g}, —SO₂NR^{g}R^{h}, halogen, —NO₂, —NR^{g}R^{h}, —(CH₂)ᵣNR^{g}R^{h}, or —C(O)—NR^{g}R^{h};
each R^{e}, R^{f}, R^{g} and R^{h} is, independently hydrogen, C₁₋₆ alkyl, C₂₋₆ alkenyl or C₁₋₆ haloalkyl,
m is 1, 2, or 3;
p is 0, 1, or 2;
q is an integer from 0 to [6+(p+2)]; and
r is 1, 2, 3 or 4;
with the proviso that the compound is not isopropyl 4′-methyl-4-((4aS,8aS)-2-oxooctahydroquinoxalin-1(2H)-yl)-1,4′-bipiperidine-1′-carboxylate, or pharmaceutically acceptable salt thereof.

64. A process for preparing a compound of claim 1, comprising reacting a compound of Formula IX, or pharmaceutically acceptable salt thereof: 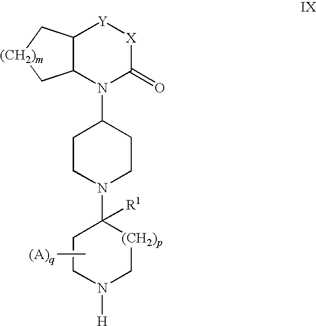
with a compound of Formula R^{b}C(O)-L², or salt thereof, wherein L² is halogen or hydroxyl, under conditions and for a time sufficient to form a compound of Formula I;
wherein:
Y is —CR³R⁴—, —NR⁵—, —O—, or —S—;
X is —CR⁶R⁷—, —NR⁸—, —O—, or —S—;
with the proviso that either Y is —CR³R⁴— or X is —CR⁶R⁷—;
each A is, independently, C₁₋₃ alkyl, or two A linked together to form a C₁₋₃alkylene bridge;
R¹ is hydrogen, C₁₋₆ alkyl, or C₁₋₆ haloalkyl;
R² is —C(O)R^{b};
R³, R⁴, R⁶, and R⁷ are each, independently, hydrogen, fluoro, C₁₋₄ alkyl, C₁₋₄ alkoxymethyl, cyanoC₁₋₄ alkyl, or C₁₋₄ haloalkyl; R⁵ and R⁸ are each, independently, hydrogen, C₁₋₄ alkyl, or C₁₋₄ haloalkyl;
R^{a}, R^{b}, R^{c}, and R^{d} are each, independently, hydrogen, C₁₋₇ alkyl, C₂₋₆ alkenyl, C₂₋₆ alkynyl, C₁₋₆ haloalkyl, C₃₋₇ cycloalkyl, C₃₋₇ cycloalkyl-C₁₋₃alkyl, C₃₋₇ heterocycloalkyl, C₃₋₇ heterocycloalkyl-C₁₋₃ alkyl, C₆₋₁₀aryl, C₆₋₁₀ aryl-C₁₋₃alkyl, C₃₋₉ heteroaryl, or C₃₋₉ heteroaryl-C₁₋₃alkyl; wherein the rings of said C₆₋₁₀aryl, C₆₋₁₀aryl-C₁₋₃alkyl, C₃₋₉ heteroaryl, and C₃₋₉ heteroaryl-C₁₋₃alkyl are each optionally substituted by 1, 2, 3, or 4 independently selected R¹² groups; wherein the rings of said C₃₋₇ cycloalkyl, C₃₋₇ cycloalkyl-C₁₋₃alkyl, C₃₋₇ heterocycloalkyl, and C₃₋₇ heterocycloalkyl-C₁₋₃ alkyl, are each optionally substituted by 1, 2, 3, or 4 independently selected R¹³ groups; and wherein said C₁₋₇ alkyl, C₂₋₆ alkenyl, C₂₋₆ alkynyl, C₁₋₆ haloalkyl, C₁₋₇ alkoxy, and C₁₋₆ haloalkoxy are each optionally substituted by 1, 2, or 3 independently selected R¹⁴ groups;
each R¹², R¹³, and R¹⁴ is, independently, phenyl, C₃₋₆cycloalkyl, C₂₋₅ heterocycloalkyl, C₃₋₅ heteroaryl, —CN, —SR^{g}, —OR^{g}, —O(CH₂)ᵣ—OR^{g}, R^{g}, —C(O)—R^{g}, —CO₂R^{g}, —SO₂R^{g}, —SO₂NR^{g}R^{h}, halogen, —NO₂, —NR^{g}R^{h}, —(CH₂)NR^{g}R^{h}, or —C(O)—NR^{g}R^{h};
each R^{e}, R^{f}, R^{g} and R^{h} is, independently hydrogen, C₁₋₆ alkyl, C₂₋₆ alkenyl or C₁₋₆ haloalkyl,
m is 1, 2, or 3;
p is 0, 1, or 2;
q is an integer from 0 to [6+(p+2)]; and
r is 1, 2, 3 or 4;
with the proviso that the compound is not isopropyl 4′-methyl-4-((4aS,8aS)-2-oxooctahydroquinoxalin-1(2H)-yl)-1,4′-bipiperidine-1′-carboxylate, or pharmaceutically acceptable salt thereof.

65. A compound of Formula IX: 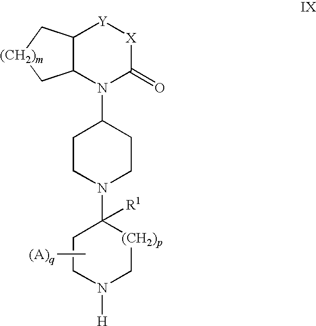
or pharmaceutically acceptable salt thereof;
wherein:
Y is —CR³R⁴—, —NR⁵—, —O—, or —S—;
X is —CR⁶R⁷—, —NR⁸—, —O—, or —S—;
with the proviso that either Y is —CR³R⁴— or X is —CR⁶R⁷—;
each A is, independently, C₁₋₃ alkyl, or two A linked together to form a C₁₋₃alkylene bridge;
R¹ is hydrogen, C₁₋₆ alkyl, or C₁₋₆ haloalkyl;
R³, R⁴, R⁶, and R⁷ are each, independently, hydrogen, fluoro, C₁₋₄ alkyl C₁₋₄ alkoxymethyl, cyanoC₁₋₄ alkyl or C₁₋₄ haloalkyl; R⁵ and R⁸ are each, independently, hydrogen, C₁₋₄ alkyl or C₁₋₄ haloalkyl;
m is 1, 2, or 3;
p is 0, 1, or 2; and
q is an integer from 0 to [6+(p+2)];
with the proviso that the compound is not isopropyl 4′-methyl-4-((4aS,8aS)-2-oxooctahydroquinoxalin-1(2H)-yl)-1,4′-bipiperidine-1′-carboxylate, or pharmaceutically acceptable salt thereof.
